(19) United States
(12) Patent Application Publication
(10) Pub. No.: US 2022/0257722 A1
(43) Pub. Date: Aug. 18, 2022

(54) COMPOSITIONS AND METHODS OF TREATING AND PREVENTING SYSTEMIC COMPLICATIONS OF ACUTE ILLNESS

(71) Applicants: United States Government As Represented By The Department of Veterans Affairs, Washington, DC (US); THE REGENTS OF THE UNIVERSITY OF CALIFORNIA, Oakland, CA (US)

(72) Inventors: Jonathan D. Kaunitz, Santa Monica, CA (US); Yasutada Akiba, Santa Monica, CA (US); Suwan Oh, Los Angeles, CA (US)

(21) Appl. No.: 17/442,831

(22) PCT Filed: Mar. 27, 2020
(86) PCT No.: PCT/US2020/025272

§ 371 Date: Sep. 24, 2021

Related U.S. Application Data
(60) Provisional application No. 62/825,087, filed on Mar. 28, 2019.

(51) Int. Cl. A61K 38/26; C07K 14/605

(52) U.S. Cl. CPC A61K 38/26; C07K 14/605

(57) ABSTRACT

The disclosure relates to compositions and methods of suppressing or preventing systemic organ inflammation or multi organ failure in a human patient with acute illnesses, including but not limited to burns, major surgery, sepsis, autoimmune disorders, vasculitis, thromboembolism, trauma, and acute pancreatitis. The method comprises administering to a patient in need of treatment an effective amount of a glucagon-like peptide-2 or an analog thereof.

### CROSS REFERENCE TO RELATED APPLICATIONS

This application claims the benefit of priority of U.S. Provisional Application No. 62/825,087, filed Mar. 28, 2019. The content of this earlier filed application is hereby incorporated by reference herein in its entirety.

### INCORPORATION OF THE SEQUENCE LISTING

The present application contains a sequence listing that is submitted via EFS-Web concurrent with the filing of this application, containing the file name “37759 0168P1 Sequence Listing.txt” which is 20,480 bytes in size, created on Mar. 26, 2020, and is herein incorporated by reference in its entirety pursuant to 37 C.F.R. § 1.52(e)(5).

### BACKGROUND

Acute pancreatitis (AP) is a common reason for hospitalization worldwide. Although the primary pathology is inflammation of the pancreas, usually due to excessive alcohol consumption, obstructing gallstones, or severe hyperlipidemia, the most feared complication of AP is multi-organ failure (MOF), a condition associated with failure of the lungs, kidneys, liver, and other organs, which has few effective treatments and is fatal in many cases. Furthermore, other acute illnesses such as burns, major surgery, sepsis, autoimmune disorders, vasculitis, thromboembolism, and trauma are also associated with the development of MOF. Thus, a need exists for treating and preventing multi-organ failure in subjects with AP and other acute illnesses associated with MOF.

### SUMMARY

Disclosed herein are methods of suppressing or preventing extrapancreatic organ inflammation in a human patient with acute pancreatitis, the methods comprising: (a) identifying a human patient in need of treatment; and (b) administering to the human patient a therapeutically effective amount of a pharmaceutical composition comprising glucagon-like peptide (GLP)-2 or a GLP-2 analog; and a pharmaceutically acceptable carrier.

Disclosed herein are methods of suppressing or preventing extrapancreatic organ inflammation in a human patient with an acute inflammatory disorder associated with systemic inflammatory response syndrome, the methods comprising: (a) identifying a human patient in need of treatment; and (b) administering to the human patient a therapeutically effective amount of a pharmaceutical composition comprising glucagon-like peptide (GLP)-2 or a GLP-2 analog; and a pharmaceutically acceptable carrier.

Disclosed herein are methods of suppressing or preventing extrapancreatic organ inflammation in a human patient with multiorgan failure, the methods comprising: (a) identifying a human patient in need of treatment; and (b) administering to the human patient a therapeutically effective amount of a pharmaceutical composition comprising glucagon-like peptide (GLP)-2 or a GLP-2 analog; and a pharmaceutically acceptable carrier.

Disclosed herein are methods of preventing or reducing endotoxin entry in a subject's lung or liver, the methods comprising: (a) identifying a human patient in need of treatment; and (b) administering to the human patient a therapeutically effective amount of a pharmaceutical composition comprising GLP-2 or a GLP-2; and a pharmaceutically acceptable carrier.

Disclosed herein are methods of preventing a systemic inflammatory response or multi-organ failure in a human patient with acute pancreatitis, the methods comprising: (a) identifying a human patient in need of treatment; and (b) administering to the human patient a therapeutically effective amount of a pharmaceutical composition comprising GLP-2 or a GLP-2 analog; and a pharmaceutically acceptable carrier.

Disclosed herein are methods of ameliorating one or more symptoms of acute pancreatitis in a subject, the method comprising: a) identifying a subject in need thereof; and b) administering to the subject a therapeutically effective amount of GLP-2 or a GLP-2 analog.

Disclosed herein are methods of ameliorating one or more symptoms of an acute illness, in a subject, the methods comprising: a) identifying a subject in need thereof; and b) administering to the subject a therapeutically effective amount of GLP-2 or a GLP-2 analog.

Disclosed herein are methods of suppressing or preventing systemic organ inflammation in a human patient with acute illness, the methods comprising: (a) identifying a human patient in need of treatment; and (b) administering to the human patient a therapeutically effective amount of a pharmaceutical composition comprising glucagon-like peptide (GLP)-2 or a GLP-2 analog; and a pharmaceutically acceptable carrier.

Disclosed herein are methods of suppressing or preventing systemic organ inflammation in a human patient with an acute inflammatory disorder associated with systemic inflammatory response syndrome (SIRS), the methods comprising: (a) identifying a human patient in need of treatment; and (b) administering to the human patient a therapeutically effective amount of a pharmaceutical composition comprising glucagon-like peptide (GLP)-2 or a GLP-2 analog; and a pharmaceutically acceptable carrier.

Disclosed herein are methods of suppressing or preventing systemic organ inflammation in a human patient with multiorgan failure, the methods comprising: (a) identifying a human patient in need of treatment; and (b) administering to the human patient a therapeutically effective amount of a pharmaceutical composition comprising glucagon-like peptide (GLP)-2 or a GLP-2 analog; and a pharmaceutically acceptable carrier.

Disclosed herein are methods of preventing a systemic inflammatory response or multi-organ failure in a human patient with an acute illness, the methods comprising: (a) identifying a human patient in need of treatment; and (b) administering to the human patient a therapeutically effective amount of a pharmaceutical composition comprising GLP-2 or a GLP-2 analog; and a pharmaceutically acceptable carrier.

Disclosed herein are method of preventing or reducing endotoxin entry into a human patient's portal vein, the methods comprising: (a) identifying the human patient in need of treatment; and (b) administering to the human patient a therapeutically effective amount of a pharmaceutical composition comprising GLP-2 or a GLP-2 analog; and a pharmaceutically acceptable carrier.

Other features and advantages of the present compositions and methods are illustrated in the description below, the drawings, and the claims.

### BRIEF DESCRIPTION OF THE DRAWINGS

FIG. 1 shows increased plasma lipase activity after cerulein treatment to induce experimental acute pancreatitis.

FIGS. 2A-C show the systemic expression of proinflammatory cytokines, tumor necrosis factor (TNF)-α (FIG. 2A), interleukin (IL)-1β (FIG. 2B) and IL-6 (FIG. 2C), in the terminal ileum (TI), liver (L), lung (Lu) and pancreas (P) 24 hours after the final intraperitoneal injection of cerulein, and after intraperitoneal injection of teduglutide (TDG).

FIG. 3 shows portal vein (PV) lipopolysaccharide (LPS) concentrations 0-24 hr after the final injection of cerulein and the decreased PV LPS concentrations after administration of teduglutide (TDG).

FIG. 4 shows increased uptake of the paracellular permeability marker FITC-dextran 4000 (FD4) from the small intestine 18 or 24 hrs after cerulein treatment, suggesting that LPS entry occurs after a pancreatitis-associated intestinal permeability increase. TDG treatment significantly reduced PV FD4 levels (FIGS. 4) 18 and 24 hrs after cerulein treatment.

FIG. 5 shows plasma TNFα levels, indicative of systemic inflammation, were increased 24 hrs after cerulein treatment, reversed by TDG treatment.

FIGS. 6A-G show LPS transport in rat jejunal mucosa in the Ussing chamber. Muscle-stripped mucosa-submucosa preparation of rat jejunal mucosa was exposed to mucosal LPS (10 μg/ml) at t=0 min, followed by the mucosal addition of vehicle (phosphate buffered saline pH 7.4) or oleic acid (OA) and taurocholic acid (TCA) at t=15 min. Serosal LPS concentrations ([LPS]) were measured using the limulus amebocyte lysate test with colorimetric detection. Background [LPS] at t=0 was subtracted from the value at each time point, which is expressed as Δ[LPS] (EU/ml) m-to-s (mean±SEM, n=6). The data were analyzed by two-way ANOVA, followed by Tukey's multiple comparisons test. FIG. 6A shows the mucosa was exposed to mucosal LPS alone for 15 min, followed by mucosal addition of vehicle (veh), TCA (0.1 mM) alone or OA (3-30 mM) with TCA. OA in the mucosal bath dose-dependently increased serosal [LPS] at t=30 and 45 min, whereas LPS alone or LPS+TCA had no effect on serosal [LPS]. *p <0.05 vs. LPS+vehicle group, †p<0.05 vs. LPS+TCA group. FIG. 6B shows vehicle was added into the mucosal bath without LPS, followed by addition of vehicle or OA/TCA. FIG. 6C shows sulfosuccinimidyl oleate (SSO, 0.1 mM) was added into the mucosal bath 10 min before LPS application, followed by addition of vehicle or OA (30 mM)/TCA (0.1 mM). SSO inhibited OA/TCA-induced [LPS] increase, whereas SSO with LPS alone had no effect on serosal [LPS]. *p<0.05 vs. LPS+vehicle group, †p<0.05 vs. LPS+OA/TCA group. FIG. 6D shows methyl-β-cyclodextrin (MβCD, 1 mM) was added into the mucosal bath 10 min before LPS application. MβCD abolished OA/TCA-induced [LPS] increase, whereas MβCD with LPS alone had no effect on serosal [LPS]. *p<0.05 vs. LPS+vehicle group, †p<0.05 vs. LPS+OA/TCA group. FIG. 6E shows carbachol (CCh, 10 μM) was added into the serosal (s) bath 10 min before LPS application. CCh increased serosal [LPS], regardless the presence of mucosal OA/TCA. *p<0.05 vs. LPS+vehicle group, †p<0.05 vs. LPS+OA/TCA group. FIG. 6F shows that glycerol phosphate (GP, 10 mM) was added into the mucosal bath 10 min before LPS application. GP enhanced OA/TCA-induced [LPS] increase at t=45 min, whereas GP with LPS alone had no effect on serosal [LPS]. *p<0.05 vs. LPS+vehicle group, †p<0.05 vs. LPS+OA/TCA group. FIG. 6G shows the effects of SSO, MβCD, CCh and GP on serosal [LPS] at t=15 min (LPS alone exposure) and at t=45 min (LPS+OA+TCA exposure). *p<0.05 vs. vehicle group, †p<0.05 vs. the corresponding LPS alone group.

FIGS. 7A-B show the comparison of LPS measurements using FITC-LPS and TLR4 reporter cell assay. Rat jejunal mucosae were exposed to mucosal FITC-LPS (10 μg/ml) with or without mucosal (m) sulfosuccinimidyl oleate (SSO, 0.1 mM) or methyl-β-cyclodextrin (MβCD, 1 mM) 10 min before FITC-LPS application, followed by mucosal addition of vehicle or OA (30 mM)/TCA (0.1 mM) at t=15 min. Serosal [LPS] at each time point was detected by FITC fluorescence intensity, followed by subtraction of background [LPS] at t=0 (FIG. 7A) or mTLR4-SEAP reporter cell assay (see Methods for detail) (FIG. 7B), expressed as Δ[FITC-LPS] (ng/ml) m-to-s, or Δ[LPS] (ng/ml) m-to-s, respectively (mean±SEM, n=6). The data were analyzed by two-way ANOVA, followed by Tukey's multiple comparisons test. *p<0.05 vs. FITC-LPS alone group, †p<0.05 vs. FITC-LPS+OA/TCA group.

FIGS. 8A-D show the epithelial permeability during lipid exposure in rat jejunal mucosa. Rat jejunal mucosa were exposed to mucosal FITC-LPS (10 μg/ml) or FITC-dextran 4000 (FD4, 0.1 mM) with LPS (10 μg/ml) at t=0 min, followed by the mucosal (m) addition of vehicle (phosphate buffered saline pH 7.4; PBS), or OA (30 mM) and TCA (0.1 mM) at t=15 min. FITC-LPS transport (m-to-s) (FIG. 8A) or FD4 transport (m-to-s) (FIG. 8C) is expressed as A[FITC-LPS] (ng/ml) m-to-s, or Δ[FD4] (nM) m-to-s, respectively (mean±SEM, n=4). Transepithelial electrical resistance (TEER) (Ω·cm²), recorded throughout the experiments is plotted every 15 min (FIGS. 8B, D) for the corresponding experiments (FIGS. 8A, C). The data were analyzed by two-way ANOVA, followed by Tukey's multiple comparisons test. FIGS. 8A, B show that luminal PBS alone or FITC-LPS alone had no effect on serosal [FITC-LPS], whereas addition of OA/TCA in the mucosal bath increased serosal [FITC-LPS] (FIG. 8A) with no change in TEER (B). *p<0.05 vs. PBS alone group, †p<0.05 vs. FITC-LPS alone group. FIGS. 8C, D show that Luminal LPS alone had no effect on serosal fluorescence change.

Luminal addition of FD4, with or without further addition of OA/TCA in the mucosal bath, and increased serosal [FD4] (FIG. 8C) with any change in TEER (D). *p<0.05 vs. LPS alone group.

FIGS. 9A-H shows the effect of GLP-2 on LPS transport during lipid exposure in rat jejunal mucosa. Rat jejunal mucosae were exposed to mucosal LPS (10 μg/ml) at t=0 min, followed by the mucosal addition of vehicle (veh) or OA and TCA at t=15 min. LPS transport (m-to-s) is expressed as Δ[LPS] (EU/ml) m-to-s (mean±SEM, n=6). Each inhibitor (D-G) was added in the serosal solution 5 min before addition of GLP-2 and NVP. The data were analyzed by two-way ANOVA, followed by Tukey's multiple comparisons test. FIG. 9A shows rat GLP-2 (100 nM) with or without NVP-728 (NVP, 10 μM) was added into the serosal(s) bath 10 min before LPS application, followed by addition of vehicle or OA (30 mM)/TCA (0.1 mM) into the mucosal (m) bath. GLP-2 with or without NVP had no effect on serosal [LPS] at t=15 min. GLP-2 with NVP inhibited OA/TCA-induced [LPS] increase, whereas GLP-2 alone had no effect on OA/TCA-induced [LPS] increase. *p<0.05 vs. LPS+vehicle group, †p<0.05 vs. LPS +OA/TCA group. FIG. 9B shows teduglutide (TDG, 100 nM) was added into the serosal(s) bath 10 min before LPS application, followed by addition of vehicle or OA/TCA. TDG inhibited OA/TCA-induced [LPS] increase. *p<0.05 vs. LPS+vehicle group, †p<0.05 vs. LPS+OA/TCA group. FIG. 9C shows that GLP-2(3-33) (300 nM) or NVP (10 μM) was added into the serosal (s) bath 10 min before LPS application, followed by addition of vehicle or OA/TCA. GLP-2(3-33) or NVP had no effect on OA/TCA-induced [LPS] increase. *p<0.05 vs. LPS+vehicle group. FIG. 9D shows that pretreatment with NVP-AEW-541 (AEW541, 10 μM, s) enhanced the OA/TCA-induced [LPS] increase. *p<0.05 vs. LPS+vehicle group, †p<0.05 vs. LPS+OA/TCA group, ‡p<0.05 vs. LPS+OA/TCA +NVP +GLP-2 group. FIG. 9E shows that pretreatment with PD153035 (1 μM, s) increased serosal [LPS] at t=15 min and further augmented OA/TCA-induced [LPS] increase. *p<0.05 vs. LPS+vehicle group, †p<0.05 vs. LPS+OA/TCA group, ‡p<0.05 vs. LPS+OA/TCA +NVP +GLP-2 group. FIG. 9F shows that pretreatment with L-NAME (0.1 mM, s) reversed the inhibitory effect of GLP-2/NVP on OA/TCA-induced [LPS] increase. *p<0.05 vs. LPS+vehicle group, †p<0.05 vs. LPS+OA/TCA group, ‡p<0.05 vs. LPS+OA/TCA +NVP +GLP-2 group. FIG. 9G shows that pretreatment with PG97-269 (1 μM, s) reversed the inhibitory effect of GLP-2/NVP on OA/TCA-induced [LPS] increase. *p<0.05 vs. LPS+vehicle group, †p<0.05 vs. LPS+OA/TCA group, ‡p<0.05 vs. LPS+OA/TCA +NVP +GLP-2 group. FIG. 9H shows that the effects of NVP +GLP-2 with or without AEW541, PD153035, L-NAME and PG97-269 on serosal [LPS] at t=15 min (LPS alone exposure) and at t=45 min (LPS+OA+TCA exposure). *p<0.05 vs. vehicle group, †p<0.05 vs. NVP +GLP-2 group, ‡p<0.05 vs. the corresponding LPS alone group.

FIGS. 10A-H shows FITC-LPS transport during lipid exposure in rat small intestine in vivo. FITC-LPS (50 μg/ml) in 2-ml PBS with or without OA (30 mM) plus TCA (10 mM) was administered by intraduodenal (id) bolus perfusion (pf) at t=0 min, followed by 2-ml PBS perfusion every 30 min. Portal venous (PV) blood and mesenteric lymph were collected every 15 min. Fluorescence intensity was measured in PV plasma and lymph with measurement of lymph output to calculate FITC-LPS content in the samples after subtraction of background fluorescence intensity in the samples at t=0, which is expressed as PV FITC-LPS (ng/ml) and FITC-LPS transport into lymph (ng/15 min) (mean±SEM, n=6). Each inhibitor was perfused in 2-ml PBS 30 min before FITC-LPS perfusion, followed by co-perfusion with FITC-LPS+OA/TCA at t=0 min. Data were analyzed by two-way ANOVA (A-F) or one-way ANOVA (G, H), followed by Tukey's multiple comparisons test. FIGS. 10A, B show that intraduodenal perfusion of FITC-LPS alone had no effect on FITC-LPS appearance into the PV (FIG. 10A) and into the lymph (FIG. 10B). Addition of OA+TCA with FITC-LPS rapidly increased PV FITC-LPS content at t=15 and 30 min, followed by a decline to the basal value (FIG. 10A), whereas FITC-LPS transport to lymph gradually increased, reaching a plateau at t=60 min. Pretreatment and co-perfusion of SSO (1 mM) reduced OA/TCA-induced FITC-LPS transport into the PV (A), but had no effect on FITC-LPS transport into the lymph (FIG. 10B). *p<0.05 vs. FITC-LPS alone group, †p<0.05 vs. FITC-LPS+OA/TCA group. FIGS. 10C, D show that pretreatment and co-perfusion of MβCD (1 mM) abolished OA/TCA-induced FITC-LPS transport into the PV (FIG. 10C), but had no effect on FITC-LPS transport into the lymph (FIG. 10D). *p<0.05 vs. FITC-LPS alone group, ‡p<0.05 vs. FITC-LPS+OA/TCA group. FIGS. 10E, F show that pretreatment and co-perfusion of Pluronic-81 (PL81, 3%) delayed OA/TCA-induced FITC-LPS transport increase into the PV (FIG. 10E), and inhibited FITC-LPS transport into the lymph (FIG. 10F). *p<0.05 vs. FITC-LPS alone group, †p<0.05 vs. FITC-LPS+OA/TCA group. FIGS. 10G, H show that the area under the curve for FITC-LPS uptake for 0-90 min (AUC₀₋₉₀) into the PV (FIG. 10G) and lymph (FIG. 10H) was calculated from FIGS. 10A, C, E and from FIGS. 10B, D, F, respectively, by the trapezoidal rule. Compared with FITC-LPS alone group, OA/TCA increased AUC₀₋₉₀ of PV and lymph. MβCD treatment abolished AUC₀₋₉₀ of PV with no effect on AUC₀₋₉₀ of lymph, whereas PL81 treatment reduced AUC₀₋₉₀ of lymph with no effect on AUC₀₋₉₀ of PV. SSO treatment had no effect on AUC₀₋₉₀ of PV and lymph. *p<0.05 vs. FITC-LPS alone group, †p<0.05 vs. FITC-LPS+OA/TCA group.

FIGS. 11A-H show the effect of TDG on FITC-LPS transport during lipid exposure in rat small intestine in vivo. TDG (50 μg/kg) was injected iv 15 min before intraduodenal (id) bolus perfusion (pf) of FITC-LPS (50 μg/ml) in 2-ml PBS with OA (30 mM) plus TCA (10 mM). FITC-LPS appearance in the PV and the lymph, and lymph output are expressed as PV FITC-LPS (ng/ml), FITC-LPS transport into lymph (ng/15 min) and lymph output (μ1/15 min) (mean ±SEM, n=6), respectively. Data were analyzed by two-way ANOVA (FIGS. 11A-F) or one-way ANOVA (FIGS. 9G, H), followed by Tukey's multiple comparisons test. FIGS. 11A-C show that TDG iv injection abolished OA/TCA-induced FITC-LPS transport into the PV (FIG. 11A), but rapidly increased FITC-LPS transport into the lymph (FIG. 11B), accompanied by rapidly enhanced lymph output (FIG. 11C). L-NAME (0.1 mM) was perfused in 2-ml PBS at t=−30 min, followed by TDG iv injection at t=−15 min. Pretreatment with and co-perfusion of L-NAME reduced the inhibitory effect of TDG on OA/TCA-induced FITC-LPS transport into the PV (FIG. 11A) and into the lymph (FIG. 9B), with reversal of the reduction of TDG-induced lymphatic output increase (FIG. 11C). *p<0.05 vs. FITC-LPS+OA/TCA group, †p<0.05 vs. +TDG group. FIGS. 11D-F shows that PG97-269 (0.3 mg/kg) was iv injected 5 min before TDG iv injection at t=−15 min. Pretreatment of PG97-269 had no effect on the inhibitory effect of TDG on OA/TCA-induced FITC-LPS transport into the PV (FIG. 11D), but inhibited FITC-LPS transport into the lymph (FIG. 11E), with reduction of TDG-induced lymph output increase (FIG. 11F). *p<0.05 vs. FITC-LPS+OA/TCA group, †p<0.05 vs. +TDG group. FIGS. 9G, H show that the AUC₀₋₉₀ into the PV (FIG. 9G) and lymph (FIG. 11H) was calculated from FIGS. 11A, D and from FIGS. 11B, E, respectively, by the trapezoidal rule. Compared with FITC-LPS +OA/TCA group, TDG reduced AUC₀₋₉₀ of PV, which was reversed by L-NAME treatment (FIG. 11G). L-NAME and PG97-269 treatment reduced AUC₀₋₉₀ of lymph, whereas TDG had no significant effect on AUC₀₋₉₀ of lymph (FIG. 11H). *p<0.05 vs. FITC-LPS+OA/TCA group, t_{p<0}._{05 vs}. _{+TDG group}.

FIG. 12 shows FITC-LPS transport during lipid exposure into the portal vein in mice Intraduodenal perfusion of FITC-LPS with or without OA (30 mM) plus TCA (10 mM) was performed in anesthetized mice, followed by portal venous (PV) blood collection 15 min after perfusion. Plasma FITC-LPS levels were measured and are expressed as PV FITC-LPS (ng/ml) (mean±SEM, n=6). *p<0.05 vs. FITC-LPS alone group. Data were analyzed by the Mann-Whitney test.

FIGS. 13A-B shows the effect of LPS treatment on small intestinal paracellular permeability (6 hr study). FITC-dextran 4kDa (FD4) solution (0.1 ml, 10 ml) was intraduodenally perfused at t=0 min under anesthesia in rats of 1, 3, or 6 hr after LPS treatment (5 mg/kg, ip), or 6 hr after saline treatment (Control). FIG. 13A shows FD4 concentration in portal venous (PV) plasma (PV FD4) collected every 15 min. Background fluorescent intensity at t=0 was subtracted from the value at each time point, and calculated FD4 concentration is expressed as PV FD4 (nM) (mean±SEM, n=6). *p<0.05 vs. Control group, †p<0.05 vs. LPS 1 hr group, ‡p<0.05 vs. LPS 3 hr group. FIG. 13B shows arterial FD4 concentration at t=90 min (mean±SEM, n=6). *p<0.05 vs. Control, †p<0.05 vs. LPS 1 hr, p<0.05 vs. LPS 3 hr.

FIGS. 14A-B shows the effect of a GLP-2 receptor antagonist on LPS-induced increases in FD4 permeability. FIG. 14A shows that GLP-2 receptor antagonist GLP-2(3-33) (1 mg/kg, ip) was given immediately (0 hr) after LPS treatment. FD4 solution was perfused as indicated in FIG. 13. A: PV FD4 concentration (mean±SEM, n=6). *p<0.05 vs. Control group, †p<0.05 vs. LPS 6 hr group. FIG. 14B shows arterial FD4 concentration at t=90 min (mean±SEM, n=6). *p<0.05 vs. Control, †p<0.05 vs. LPS 6 hr.

FIGS. 15A-B shows FD4 permeability 24 hr after LPS treatment; effect of GLP-2 treatment LPS was injected (5 mg/kg, ip) 24 hr before the experiments and FD4 solution was perfused as indicated in FIG. 13. Rat GLP-2 (380 μg/kg, ip) was injected 6 hr after LPS treatment. FIG. 12A shows PV FD4 concentration (mean±SEM, n=6). *p<0.05 vs. Control group, †p<0.05 vs. LPS 24 hr group. FIG. 15B shows arterial FD4 concentration at t=90 min (mean±SEM, n=6). *p<0.05 vs. Control, †p<0.05 vs. LPS 24 hr.

FIG. 16 shows the correlation between FD4 transported into the PV and arterial FD4 concentration. Area under the curve (AUC) of PV FD4 concentration (μM⋅min) (PV FD4 AUC) calculated by the trapezoidal rule, and arterial FD4 concentration (nM) at t=90 min (Arterial FD4) from the data in FIGS. 13-15 were plotted. Linear regression was calculated by GraphPad® Prism 6 statistics software.

FIGS. 17A-B shows the effect of LPS treatment on PV GLP-2 levels. FIG. 17A shows that plasma GLP-2 content was measured in the PV blood in overnight fasted control and 6 hr after LPS treatment (LPS 6 hr). Each column is expressed as mean±SEM (n=6). *p<0.05 vs. Control (fasted). FIG. 17B shows the plasma GLP-2 content was measured in the PV blood in fed ad libitum Control and 24 hr after LPS treatment (LPS 24 hr). Each column is expressed as mean±SEM (n=6). *p<0.05 vs. Control (fed).

FIGS. 18A-I shows the effect of LPS treatment on expressions of GLP-2-related proteins and proinflammatory mediators. mRNA expressions in the ileal mucosa of overnight fasted control and 6 hr after LPS treatment (LPS 6 hr group) were assessed by real-time PCR using β-actin as internal control with ΔCT method. Each column is expressed as mean±SEM (n=6). *p<0.05 vs. Control (fasted). FIG. 18A: Proglucagon (Gcg), FIG. 18B: GLP-2 receptor (GLP2R), FIG. 18C: cyclooxygenase-2 (COX2), FIG. 18D: tumor necrosis factor α (TNFα), FIG. 18E: interleukin-6 (IL6), FIG. 18F: epidermal growth factor (EGF), FIG. 18G: insulin-like growth factor 1 (IGF1), FIG. 18H: IGF1 receptor (IGF1R), FIG. 181: IGF2R.

FIGS. 19A-G shows the effect of teduglutide on LPS-induced small intestinal FD4 permeability (6 hr model). LPS was given 6 hr before the experiments. Teduglutide (TDG, 50 μg/kg) was injected 3 hr (ip) or 6 hr (iv at t=0 min) after LPS treatment. The FD4 solution was perfused as indicated in FIG. 13. Test drugs were iv injected at t=−10 min, or co-perfused (pf) with FD4 solution. FIG. 19A shows PV FD4 concentration (mean±SEM, n=6) of Control, LPS 6hr, LPS 6 hr +TDG 3 hr and LPS 6hr +TDG 6hr group. *p<0.05 vs. Control group, †p<0.05 vs. LPS 6 hr group. FIG. 19B shows the effect of IGF1R inhibitor NVP-AEW541 (AEW541) (0.1 mg/kg, iv) on the inhibitory effect of TDG on LPS-induced PV FD4 increase (mean±SEM, n=6). *p<0.05 vs. LPS 6 hr. FIG. 19C shows the effect of EGF receptor inhibitor PD153035 (10 μg/kg, iv) on the inhibitory effect of TDG on LPS-induced PV FD4 increase (mean±SEM, n=6). *p<0.05 vs. LPS 6 hr. FIG. 19D shows the effect of VPAC1 antagonist PG97-269 (1 mg/kg, iv) on the inhibitory effect of TDG on LPS-induced PV FD4 increase (mean±SEM, n=6). *p<0.05 vs. LPS 6 hr, <0.05 vs. LPS 6 hr +TDG 6 hr. FIG. 19E shows the effect of NO synthase inhibitor L-NAME (0.1 mM, pf) on the inhibitory effect of TDG on LPS-induced PV FD4 increase (mean±SEM, n=6). *p<0.05 vs. LPS 6 hr, <0.05 vs. LPS 6 hr +TDG 6 hr. FIG. 19F shows the arterial FD4 concentration at t=90 min (mean ±SEM, n=6). *p<0.05 vs. Control, †p<0.05 vs. LPS 6 hr. ‡p<0.05 vs. LPS 6 hr +TDG 6 hr. FIG. 19G shows the effect of L-NAME (0.1 mM, pf) on LPS-induced PV FD4 increase (mean ±SEM, n=6). *p<0.05 vs. LPS 6 hr.

FIGS. 20A-B shows the effect of teduglutide on LPS-induced small intestinal FD4 permeability (24 hr model). LPS was given 24 hr before the experiments. TDG (50 μg/kg) was injected 0, 6, 12 hr (ip) or 24 hr (iv at t=0 min) after LPS treatment. The FD4 solution was perfused as indicated in FIG. 13. FIG. 20A shows the PV FD4 concentration (mean±SEM, n=6). *p<0.05 vs. Control group, †p<0.05 vs. LPS 24 hr group. FIG. 20B shows the arterial FD4 concentration at t=90 min (mean±SEM, n=6). *p<0.05 vs. Control, †p<0.05 vs. LPS 24 hr.

FIG. 21 shows the proposed mechanisms by which endogenous or exogenous GLP-2 prevents LPS-induced small intestinal paracellular permeability increase. Systemic treatment of LPS induces small intestinal inflammation via stimulation of residential immune cells such as macrophage (Mϕ) through Toll-like receptor 4 (TLR4) activation, which produce proinflammatory cytokines (tumor necrosis factor-α; TNFα, interleukin-6; IL6, etc.), those injure epithelial cells and increase paracellular spaces, that increases paracellular permeability to macromolecule FITC-dextran 4000 (FD4) as well as to luminal LPS, the latter may aggravate LPS-related inflammation. LPS directly or indirectly via cytokines induces GLP-2 release from L cells, which prevents paracellular permeability increase in early time point after LPS treatment. Exogenous GLP-2 or a stable analog teduglutide (TDG) prevents LPS-induced paracellular permeability increase up to 6 hrs in early time point or 6-12 hr after LPS treatment in 24 hr time point. Effects of GLP-2 on LPS-induced paracellular permeability increase are mediated by vasoactive intestinal peptide (VIP) release and nitric oxide (NO) production from the myenteric nerves expressing GLP-2 receptors (GLP-2R).

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### DEFINITIONS

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, the subject is a human. The term does not denote a particular age or sex. Thus, adult, child, adolescent and newborn subjects, as well as fetuses, whether male or female, are intended to be covered.

As used herein, the term “patient” refers to a subject afflicted with a disease, disorder or condition. The term “patient” includes human and veterinary subjects. In some aspects of the disclosed methods, the “patient” has been diagnosed with a need for treatment prior to the administering step.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. Treatment can also be administered to a subject to ameliorate one more signs of symptoms of a disease, disorder, and/or condition. For example, the disease, disorder, and/or condition can be relating to acute pancreatitis, extrapancreatic organ inflammation, systemic organ inflammation, multiorgan failure, systemic inflammatory response, and/or entry of endotoxin into systemic circulation.

As used herein, the term “amelioration” refers to a lessening of at least one indicator, sign, or symptom of an associated disease, disorder, or condition. The severity of indicators may be determined by subjective or objective measures, which are known to those skilled in the art.

### INTRODUCTION

Among the most feared complication of acute illnesses including but not limited to acute burns, major surgery, sepsis, autoimmune disorders, vasculitis, thromboembolism, trauma, and acute pancreatitis (AP) is multiple organ failure (MOF). MOF is believed to originate from increased amounts of endotoxin, also referred to as lipopolysaccharide (LPS), a component of the outer membrane of Gram-negative bacteria that exist in high numbers in the gut lumen, entering the portal vein (PV) and inflaming the liver. Increased gut permeability elicited by systemic inflammation resulting from acute illnesses including but not limited to acute burns, major surgery, sepsis, autoimmune disorders, vasculitis, thromboembolism, trauma, and acute pancreatitis, increases LPS uptake from the gut lumen into the PV, inflaming the liver that in turns initiates a severe pro-inflammatory cascade that results in severe systemic inflammation that produces the systemic inflammatory response syndrome (SIRS) that can eventuate in MOF. LPS is one of the most potent pro-inflammatory substances known, capable of inducing a septic-shock-like syndrome after parenteral injection into humans of minute quantities of LPS.

Glucagon-like peptide-2 (GLP-2) is a peptide hormone product of proglucagon that has many beneficial properties. GLP-2 is an intestinotrophic factor released from enteroendocrine L cells, present in the gut epithelium, that increases intestinal epithelial stem cell proliferation when given chronically but also acutely increases the transport of long-chain fatty acids from the gut lumen into the lymphatic system and strengthens intestinal barrier function. A stable GLP-2 analog (teduglutide) is approved for the treatment of the short gut syndrome, usually defined as inability to assimilate adequate amounts of fluid, nutrients, and electrolytes due to gut malfunction or extensive resection, due to its pro-proliferative effects on gut epithelial stem cells. Less commonly known are the acute effects of GLP-2 on LPS transport from the gut.

As disclosed herein, it was found that GLP-2 acutely inhibits LPS uptake from the gut lumen into the portal vein (PV). In an experimental model of acute pancreatitis, acute administration of teduglutide reduced pulmonary and hepatic inflammation to near normal concentrations by preventing the increase of LPS concentrations in the PV blood. Disclosed herein are methods of administering glucagon-like peptide (GLP)-2 or a GLP-2 analog (e.g. teduglutide) acutely in subjects diagnosed or suspected of having acute pancreatitis to strengthen the gut mucosal barrier and limit LPS uptake from the gut to the PV by shunting LPS uptake to the lymphatic system, thereby preventing or reducing the severity of the morbid systemic complications of severe acute illnesses including but not limited to acute burns, major surgery, sepsis, autoimmune disorders, vasculitis, thromboembolism, trauma, and acute pancreatitis, and thus reducing the overall morbidity and mortality of the diseases.

### COMPOSITIONS

Cleavage of proglucagon produces GLP-2 and GLP-1. GLP-2 is produced by the intestinal endocrine L cell and by neurons in the central nervous system, and is secreted in the intestines upon nutrient ingestion. In humans, GLP-2 has the sequence HADGSFSDEMNTILDNLAARDFINWLIQTKITD (SEQ ID NO: 1) or HADGSFSDEMNTILDNLAARDFINWLIQTKITDR (SEQ ID NO: 2). As used herein, GLP-2 can refer to the human sequence (SEQ ID NO: 1). In some aspects, GLP-2 can refer to mouse, rat, non-human primate or other species GLP-2 sequences. It is within one of ordinary skill in the art to identify peptides with sequences that function as described herein. U.S. Pat. No. 5,789,379 discloses GLP-2-related peptides including GLP-2 analogs. In some aspects, the GLP-2 peptides disclosed in U.S. Pat. No. 5,789,379 can be useful in any of the methods disclosed herein. As such, U.S. Pat. No. 5,789,379 is hereby incorporated herein in its entirety.

GLP-2 and GLP-2 analogs can be synthesized using standard techniques of peptide chemistry. GLP-2 and GLP-2 analogs can be produced in commercial quantities by application of recombinant technology. Examples of GLP-2 analogs are disclosed in U.S. Pat. No. 5,789,379 and are incorporated herein by reference. In some aspects, the GLP-2 analogs can be purified using any standard approach. In some aspects, the GLP-2 or GLP-2 analog can be treated to exchange the cleavage acid (e.g., TFA) with a pharmaceutically acceptable salt, such as acetic, hydrochloric, phosphoric, maleic, tartaric, succinic and the like, to generate a pharmaceutically acceptable acid salt of the peptide.

In some aspects, a GLP-2 or GLP-2 analog can be a fragment. In some aspects, the fragment can be a truncated GLP-2 peptide. For example, the truncated GLP-2 peptide (amino acids 3-33) can be missing the first 2 amino acids. This peptide is a weak agonist and a partial antagonist of GLP-2. In some aspects, that the entire sequence GLP-2 or GLP-2 analog may be needed for full receptor binding.

In some aspects, the GLP-2 analog can be selected from Table 1.

TABLE 1
GLP-2 analogs.

| | | SEQ ID |
|---|---|---|
| Name | Sequence | NO: |
| rGLP-2 | HADGSFSDEMNTILDNLATRDFINWLIQTKITD | 3 |
| Tyr1 rGLP-2 | YADGSFSDEMNTILDNLATRDFINWLIQTKITD | 4 |
| D-Ala2 rGLP-2 | HADGSFSDEMNTILDNLATRDFINWLIQTKITD | 5 |
| | (D-Ala2) | |
| Gly2 rGLP-2 | HGDGSFSDEMNTILDNLATRDFINWLIQTKITD | 6 |
| Val2 rGLP-2 | HVDGSFSDEMNTILDNLATRDFINWLIQTKITD | 7 |
| Gly2 hGLP-2 | HGDGSFSDEMNTILDNLAARDFINWLIQTKITD | 8 |
| (Teduglutide) | | |
| Gly2 Ala20 hGLP-2 | HGDGSFSDEMNTILDNLAAADFINWLIQTKITD | 9 |
| Gly2 Ala10 hGLP-2 | HGDGSFSDEANTILDNLAARDFINWLIQTKITD | 10 |
| Ala1 Gly2 hGLP-2 | AGDGSFSDEMNTILDNLAARDFINWLIQTKITD | 11 |
| Gly2 Ala3 hGLP-2 | HGAGSFSDEMNTILDNLAARDFINWLIQTKITD | 12 |
| Gly2 Ala4 hGLP-2 | HGDASFSDEMNTILDNLAARDFINWLIQTKITD | 13 |
| Glu3 rGLP-2 | HAEGSFSDEMNTILDNLATRDFINWLIQTKITD | 14 |
| Ala4 rGLP-2 | HADASFSDEMNTILDNLATRDFINWLIQTKITD | 15 |
| Tyr9Ser10Lys11Tyr12 | HADGSFSDYSKYILDNLAARDFINWLIQTKITD | 16 |
| (desIle13) hGLP-2 | (I1e13 = desIle) | |
| Leu10 rGLP-2 | HADGSFSDELNTILDNLATRDFINWLIQTKITD | 17 |
| Nleu10 rGLP-2 | HADGSFSDELNTILDNLATRDFINWLIQTKITD | 18 |
| | (Nleu10) | |
| Met SO210 rGLP-2 | HADGSFSDEMNTILDNLATRDFINWLIQTKITD | 19 |
| | (Met SO210) | |
| Lys20 rGLP-2 | HADGSFSDEMNTILDNLATKDFINWLIQTKITD | 20 |
| Val23 Gln24 hGLP-2 | HADGSFSDEMNTILDNLAARDFVQWLIQTKITD | 21 |
| Amidated C-term | HADGSFSDEMNTILDNLAARDFINWLIQTKITD | 22 |
| | (D33-NH2) | |
| Gly2 Ala24 hGLP-2 | HGDGSFSDEMNTILDNLATRDFIAWLIQTKITD | 23 |
| Gly2 Ala8 hGLP-2 | HGDGSFSAEMNTILDNLATRDFINWLIQTKITD | 24 |
| Gly2 Ala11 hGLP-2 | HGDGSFSDEMATILDNLATRDFINWLIQTKITD | 25 |
| Gly2 Ala21 hGLP-2 | HGDGSFSDEMNTILDNLATRAFINWLIQTKITD | 26 |
| Gly2 Ala9 hGLP-2 | HGDGSFSDAMNTILDNLATRDFINWLIQTKITD | 27 |
| Gly2 Ala16 hGLP-2 | HGDGSFSDEMNTILDALATRDFINWLIQTKITD | 28 |
| Gly2 Ala17 hGLP-2 | HGDGSFSDEMNTILDNAATRDFINWLIQTKITD | 29 |
| Gly2 Ala28 hGLP-2 | HGDGSFSDEMNTILDNLATRDFINWLIATKITD | 30 |
| Gly2 Ala5 hGLP-2 | HGDGAFSDEMNTILDNLATRDFINWLIQTKITD | 31 |
| Gly2 Ala31 hGLP-2 | HGDGSFSDEMNTILDNLATRDFINWLIQTKATD | 32 |
| Gly2 Ala27 hGLP-2 | HGDGSFSDEMNTILDNLATRDFINWLAQTKITD | 33 |
| Gly2 Ala12 hGLP-2 | HGDGSFSDEMNAILDNLATRDFINWLIQTKITD | 34 |
| Gly2 Ala13 hGLP-2 | HGDGSFSDEMNTALDNLATRDFINWLIQTKITD | 35 |
| Gly2 Ala7 hGLP-2 | HGDGSFADEMNTILDNLATRDFINWLIQTKITD | 36 |
| Gly2 Ala6 hGLP-2 | HGDGSASDEMNTILDNLATRDFINWLIQTKITD | 37 |
| hGLP-2 | HADGSFSDEMNTILDNLAARDFINWLIQTKITD | 38 |
| (Gly2 Nle10 D-Phe11 | HGDGSFSDENleFTILDLLAARDFINWLIQTKITD | 39 |
| Leu16 hGLP-2 | | |
| (Apraglutide) | | |
| Glepaglutide | HGEGTFSSELATILDALAARDFIAWLIATKITDK | 40 |
| | KKKKK | |

As disclosed herein, GLP-2 and GLP-2 analogs can be useful for suppressing or preventing organ inflammation (e.g., extrapancreatic and/or systemic) and/or multi-organ failure in patients with illnesses that are associated with SIRS and MOF, including but not limited to burns, major surgery, sepsis, autoimmune disorders, vasculitis, thromboembolism, trauma, and acute pancreatitis. As disclosed herein, GLP-2 and GLP-2 analogs can be useful for preventing the systemic inflammatory response in patients with one or more of these illnesses. Further disclosed herein, GLP-2 and GLP-2 analogs can be useful for preventing or reducing endotoxin entry into the PV, which is believed to generate the systemic inflammatory response by activating Kupffer cells in the liver. Currently, there are no established effective treatments for suppressing or preventing multiple organ failure or SIRS in patients with acute illness. No specific treatments other than supportive care e.g., IV fluids, mechanical ventilation, therapeutic agents including vasopressors and antibiotics are available. Teduglutide, also known as Gattex™ and Revestive™, is a GLP-2 analog. The elimination half-life of teduglutide in humans is about 2 hours. Teduglutide differs from GLP-2 by a single amino acid substitution; an alanine in position 2 is replaced with a glycine. This amino acid change blocks dipeptidyl peptidase from breaking down the molecule and is responsible for increasing its half-life to 2 hours. In some aspects, other treatments can be IV fluids, mechanical ventilation, therapeutic agents including vasopressors, antibiotics or a combination thereof.

Any method known to one of ordinary skill in the art can be used to determine if a particular response is induced. Clinical methods that can assess the degree of a particular disease state can be used to determine if a response is induced. For example, in a patient with acute pancreatitis, clinical methods can include the assessment of vital signs, tissue perfusion and oxygenation, and renal, hepatic, and central nervous system functioning in order to assess the presence and/or severity of one or more symptoms of SIRS as well as one or more signs or symptoms of multiple organ failure. Restoration of abnormal vital signs to normal, and of tissue perfusion and oxygenation, and renal, hepatic, and central nervous system functioning to normal can be used as evidence that MOF and/or SIRS have responded to treatment.

SIRS and MOF are not limited to severe acute pancreatitis. SIRS and/or MOF can complicate many acute illnesses, including but not limited to burns, major surgery, sepsis, autoimmune disorders, vasculitis, thromboembolism, trauma, and acute pancreatitis. The signs and symptoms of SIRS include but are not limited to increased heart rate, abnormally low or high body temperature, increased peripheral white blood cell count, and low blood pressure, as recently reviewed Kaukonen K M, Bailey M, Pilcher D, Cooper D J, Bellomo R. Systemic inflammatory response syndrome criteria in defining severe sepsis. N Engl J Med. 2015 Apr. 23; 372(17):1629-38.

In some aspects, the methods to determine if a particular response is induced can include comparing a patient's sample with standard reference concentrations for a particular marker or assay. Standard reference concentrations can typically represent the concentrations derived from a large population of individuals. The reference population may include individuals of similar age, body size; ethnic background or general health as the subject in question. Thus, for example, marker concentrations in a patient's sample can be compared to values derived from: subjects who have not received GLP-2 or a GLP-2 analog; subjects who have successfully received GLP-2 or a GLP-2 analog, i.e., subjects who have successfully recovered from acute pancreatitis or other acute illness; and/or subjects who are suffering from acute pancreatitis or other acute illness. Any population size can be used to determine the reference concentrations. For example, a population of between 1 and 250, e.g., 1, 2, 3, 4, 5, 10, 15, 20, 25, 30, 40, 50, 100, 150, 200, 250 or more subjects can be used to determine the average reference concentrations, with greater accuracy in the measurement coming from larger sample populations. In some aspects, the marker can be circulating LPS, LPS antibodies, or one or more circulating cytokines. In some aspects, the one or more cytokines can be tumor necrosis factor alpha, interleukin-1(3 or interleukin-6.

The time period of therapeutic effectiveness of GLP-2 or a GLP-2 analog from a single (or multiple) dose(s) administration can last from about 2 minutes to over 2 hours. In some aspects, a time period of therapeutic effectiveness of GLP-2 or a GLP-2 analog can be between 2 minutes to 5 minutes or 5 minutes to 2 hours respectively. In some aspects, the time period of therapeutic effectiveness of GLP-2 or a GLP-2 analog can be at least 1 minute, 1 hour, at least 2 hours, at least 3 hours or at least 4 hours or any time period in between. GLP-2 or a GLP-2 analog can induce physiological responses such as altering intestinal motility, increasing the rate of intestinal mucosal anion secretion, and increasing the transport of long chain fatty acids from intestinal lumen to the lymphatics within 1-20 minutes. These effects can last up to 5 minutes with GLP-2 but can last 2 hours or longer with GLP-2 analogs.

Generally, the therapeutic effectiveness of GLP-2 or of GLP-2 analogs in preventing the systemic inflammatory response to acute illness can be considered maximal early in the disease course, generally regarded as within the initial 2-3 days following disease onset. In some aspects, the therapeutic effectiveness can be increased by administering the GLP-2 or the GLP-2 analogs early, for example, as soon as possible before the induction of systemic inflammation. The administration of GLP-2 or of GLP-2 analogs should start as soon as possible following the onset of acute illness, including, but not limited to burns, major surgery, sepsis, autoimmune disorders, vasculitis, thromboembolism, trauma, acute pancreatitis or a combination thereof.

In some aspects, the GLP-2 or GLP-2 analogs can be used in combination with other therapies used in the methods disclosed herein. For example, in some aspects, GLP-2 or GLP-2 analogs can be administered before, after or concurrently with intravenous rehydration therapy, antibiotics, vasopressors, mechanical ventilation, and other life-support measures.

Duration of the treatment with GLP-2 or a GLP-2 analog as disclosed herein can be any length of time as short as 1 s, 10 s, 15 s, 30 s, 40 s, 50 s, or 60 s to as long as 1 month, 2 months, 3 months, 5 months or 6 months. In some aspects, the treatment with GLP-2 and a GLP-2 analog as disclosed herein can be 1 minute, 5 minutes, 10 minutes, 15 minutes, 30 minutes, 1 hour, 2 hours, 3 hours, 4 hours, 5 hours, 10 hours, 15 hours, 20 hours, 21 hours, 22 hours, 23 hours, 24 hours, 2 days, 3 days, 4 days, 5 days, 10 days, 15, days, 20 days, 30 days, 2 months, 3 months, 4 months, 5 months, 6 months or any time in between or longer. For example, GLP-2 and a GLP-2 analog can be administered 1 second, 2 seconds, 3 seconds, 4 seconds, 5 seconds, 10 seconds, 15 seconds, 30 seconds, 1 minute, 5 minutes, 10 minutes, 15 minutes, 30 minutes, 1 hour, 2 hours, 3 hours, 4 hours, 5 hours, 10 hours, 15 hours, 20 hours, 21 hours, 22 hours, 23 hours, 24 hours or any time (seconds, minutes, hours) in between before the administration of rehydration therapy. The frequency of the treatment can vary. In some aspects, the initial administration of GLP-2 and a GLP-2 analog can precede the initial administration of rehydration therapy by 1 second, 2 seconds, 3 seconds, 4 seconds, 5 seconds, 10 seconds, 15 seconds, 30 seconds, 1 minute, 5 minutes, 10 minutes, 15 minutes, 30 minutes, 1 hour, 2 hours, 3 hours, 4 hours, 5 hours, 10 hours, 15 hours, 20 hours, 21 hours, 22 hours, 23 hours, 24 hours or any time (seconds, minutes, hours) in between or longer. In some aspects, the subsequent administration(s) of GLP-2 and a GLP-2 analog can be for part of or for the whole duration of the days that the subject receives rehydration therapy, antibiotics, vasopressors, mechanical ventilation, and other life-support measures. In some aspects, the duration of the administration of the GLP-2 and a GLP-2 analog and rehydration therapy can be between 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days or longer. Once treatment with GLP-2 and a GLP-2 analog begins, GLP-2 and a GLP-2 analog can be administered to the subject in need thereof continuously or every 1 hour or less frequently by intravenous infusion or subcutaneously every 12 hours or less frequently for the duration of the rehydration therapy.

In some aspects, the treatment regimen can be administered at doses ranging between 0.05 mg/kg/day and 1 mg/kg/day of GLP-2 or a GLP-2 analog one or more times a day starting from the time of suspected or confirmed onset of any of the acute illnesses disclosed herein or surgery, burns, or trauma. In some aspects, the treatment regimen can be administered at doses ranging between .05 mg/kg/day and 1 mg/kg/day of GLP-2 or a GLP-2 analog one or more times a day. In some aspects, the treatment regimen can be administered starting at least 1 second to 12 hours before an inflammatory stimulus, before the induction of systemic inflammation or any signs or symptoms of the acute illnesses disclosed herein or surgery, burns, or trauma. In some aspects, said treatment regimen can be continued for as long as needed until one or more symptoms improve and/or resolve. In some aspects, said treatment regimen can be continued for as long as needed until one or more symptoms improve and/or resolve as they relate to or are associated with MOF, inflammation associated with SIRS or endotoxin entry into the PV. In some aspects, said treatment regimen can be continued for as long as needed until one or more symptoms improve and/or resolve as they relate to or are associated with MOF, inflammation associated with SIRS, extrapancreatic organ inflammation or endotoxin entry into the liver or lung. In some aspects, said treatment regimen can be carried out for one or more days, one or more weeks, or one or more months until the one or symptoms improve or resolve. In some aspects, 0.05 mg/kg/day to 1 mg/kg/day of GLP-2 or a GLP-2 analog can be administered continuously or every 1, 2, 4, 6, 8, 10, 12, 14, 16, 18, 20, 22, 24 hours in divided doses. In some aspects, 0.05 mg/kg/day to 1 mg/kg/day of GLP-2 or a GLP-2 analog can be administered daily. In some aspects, 0.05 mg/kg/day to 1 mg/kg/day of GLP-2 or a GLP-2 analog can be administered over a period of about 24 hours. In some aspects, 0.05 mg/kg/day to 1 mg/kg/day of GLP-2 or a GLP-2 analog can be administered over a period of at least 24 hours to about 7 days. In some aspects, 0.05 mg/kg/day to 1 mg/kg/day of GLP-2 or a GLP-2 analog can be administered through continuous intravenous infusion. In some aspects, 0.05 to 1 mg/kg/day of GLP-2 or a GLP-2 analog can be administered via a continuous intravenous infusion over a period of about 24 hours. In some aspects, 0.05 mg/kg/day to 1 mg/kg/day of GLP-2 or a GLP-2 analog can be administered through intermittent intravenous infusion. In some aspects, 0.05 mg/kg/day to 1 mg/kg/day of GLP-2 or a GLP-2 analog can be administered through intermittent intravenous infusion up to 12 times per day. In some aspects, 0.05 mg/kg/day to 1 mg/kg/day of GLP-2 or a GLP-2 analog can be administered every 0.5, 1, 2, 4, 6, 8, 10, 12, 14, 16, 18, 20, 22, 24 hours in divided doses. In some aspects, 0.05 mg/kg/day to 1 mg/kg/day of GLP-2 or a GLP-2 analog can be administered through a subcutaneous injection. In some aspects, 0.05 mg/kg/day to 1 mg/kg/day of GLP-2 or a GLP-2 analog can be administered through a subcutaneous injection once or twice per day. In some aspects, 0.05 to 1 mg/kg/day mg of GLP-2 or a GLP-2 analog can be administered over a period of every 1 hour to 24 hours or longer.

The time period of therapeutic effectiveness of GLP-2 or a GLP-2 analog from a single (or multiple) dose(s) administration can last from about 2 minutes to 2 hours. In some aspects, a time period of therapeutic effectiveness of GLP-2 or a GLP-2 analog can be between 2 minutes to 120 minutes. In some aspects, the time period of therapeutic effectiveness of GLP-2 or a GLP-2 analog can be at least 60 minutes, 1 hour, 2 hours, 3 hours, 4 hours, 5 hours, 6 hours, 7 hours, or 8 hours or any time period in between. In some aspects, the time period of therapeutic effectiveness of GLP-2 or a GLP-2 analog can be at least 9 hours, 10 hours, 11 hours, or 12, hours or any time period in between after administration.

### METHODS OF TREATMENT

Disclosed herein, are methods of suppressing or preventing systemic organ inflammation in a human patient with acute illness. Disclosed herein, are methods of suppressing or preventing systemic organ inflammation in a human patient with an acute inflammatory disorder associated with the systemic inflammatory response syndrome (SIRS). Disclosed herein, are methods of suppressing or preventing systemic organ inflammation in a human patient with multi-organ failure (MOF). Disclosed herein, are methods of suppressing or preventing extrapancreatic organ inflammation in a human patient with acute pancreatitis. Disclosed herein, are methods of suppressing or preventing extrapancreatic organ inflammation in a human patient with an acute inflammatory disorder associated with the systemic inflammatory response syndrome (SIRS). Disclosed herein, are methods of suppressing or preventing extrapancreatic organ inflammation in a human patient with multi-organ failure (MOF). In some aspects, the human patient can have one or more diseases including, but not limited to burns, major surgery, sepsis, autoimmune disorders, vasculitis, thromboembolism, trauma, acute pancreatitis or a combination thereof that are associated with an acute inflammatory disorder associated with SIRS, or MOF. In some aspects, the extrapancreatic organ inflammation or systemic organ inflammation can be multiorgan inflammation. The method can comprise: (a) identifying a human patient in need of treatment; and (b) administering to the human patient a therapeutically effective amount of a pharmaceutical composition comprising glucagon-like peptide (GLP)-2 or a GLP-2 analog; and a pharmaceutically acceptable carrier. Acute inflammatory disorders associated with SIRS include but are not limited to burns, major surgery, sepsis, autoimmune disorders, vasculitis, thromboembolism (or thromboembolic diseases), trauma, acute pancreatitis, and cytokine release syndrome.

In some aspects, systemic organ inflammation can be suppressed or prevented by increasing the entry of LPS from the intestinal lumen to the lymphatic system and decreasing its entry into the PV. In some aspects, systemic organ inflammation can be suppressed or prevented by early administration of GLP-2 or GLP-2 analogs. In some aspects, the GLP-2 or GLP-2 analogs can be administered at around 12 hours before exposure to an inflammatory stimulus or before the induction of systemic inflammation or between is to 12 hours after exposure to an inflammatory stimulus or the induction of systemic inflammation. In some aspects, the GLP-2 or GLP-2 analogs can be administered at 1 hour, 2 hours, 3 hours, 4 hours, 5 hours, 6 hours, 7 hours, 8 hours, 9 hours, 10 hours, 11 hours or 12 hours or any time in between before exposure to an inflammatory stimulus or before the induction of systemic inflammation or 1 hour, 2 hours, 3 hours, 4 hours, 5 hours, 6 hours, 7 hours, 8 hours, 9 hours, 10 hours, 11 hours or 12 hours after exposure to an inflammatory stimulus or induction of systemic inflammation. In some aspects, the GLP-2 or GLP-2 analogs can be administered at 1 s, 5 s, 10 s, 15 s, 20 s, 25 s, 30 s, 35 s, 40 s, 45 s, 50 s, 55 s, 60 s, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes or any time in between before exposure to an inflammatory stimulus or before the induction of systemic inflammation or 1 s, 5 s, 10 s, 15 s, 20 s, 25 s, 30 s, 35 s, 40 s, 45 s, 50 s, 55 s, 60 s, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes or any time in between or after exposure to an inflammatory stimulus or after the induction of systemic inflammation. In some aspects, the GLP-2 or GLP-2 analogs can be administered any time after exposure to an inflammatory stimulus or induction of systemic inflammation including 12 hours after exposure to an inflammatory stimulus or induction of systemic inflammation. In some aspects, the GLP-2 or GLP-2 analogs can be administered between 12 hours before exposure to an inflammatory stimulus or induction of systemic inflammation and 12 hours after exposure to an inflammatory stimulus or induction of systemic inflammation. In some aspects, the inflammatory stimulus can be LPS or any inflammatory stimulus known to one of ordinary skill in the art. In some aspects, extrapancreatic organ inflammation can be suppressed or prevented in the patient's liver. In some aspects, extrapancreatic organ inflammation can be suppressed or prevented in the patient's lung. In some aspects, extrapancreatic organ inflammation can be suppressed or prevented one or more organs of the patient. In some aspects, extrapancreatic organ inflammation or systemic organ inflammation can be suppressed or prevented in a patient's liver, lungs, kidneys, brain, hematopoietic system, gastrointestinal system, blood coagulation system, vascular system, other systems, or a combination thereof.

In some aspects, the extrapancreatic organ inflammation or systemic organ inflammation can be suppressed or prevented by reducing expression or preventing an increase in the expression of one or more cytokines. In some aspects, extrapancreatic inflammation and/or systemic organ inflammation can manifest by increased cytokines. In some aspects, the one or more cytokines can be systemically expressed. In some aspects, the extrapancreatic organ inflammation or systemic organ inflammation can be suppressed or prevented by redirecting LPS originating in the intestinal lumen to the lymphatic system while reducing its entry into the PV, which reduces liver inflammation, which in turn reduces the expression or prevents an increase in the expression of one or more systemic cytokines. In some aspects, the one or more cytokines can be tumor necrosis factor alpha, interleukin-1(3 or interleukin-6, and other pro-inflammatory cytokines. In some aspects, the extrapancreatic organ inflammation or systemic organ inflammation can be suppressed or prevented by reducing lipopolysaccharide concentrations in the patient's portal venous blood. In some aspects, systemic organ inflammation can be suppressed or prevented by reducing lipopolysaccharide concentrations in the patient's portal venous blood. In some aspects, the extrapancreatic organ inflammation or systemic organ inflammation can be suppressed or prevented by preventing an increase in lipopolysaccharide concentrations in the patient's systemic circulation.

Disclosed herein, are also methods of preventing multi-organ failure in a human patient with an acute illness. In some aspects, the acute illness can be burns, major surgery, sepsis, autoimmune disorders, vasculitis, thromboembolism, trauma, and acute pancreatitis. Disclosed herein, are also methods of preventing multi-organ failure in a human patient with acute pancreatitis. Disclosed herein are methods of preventing systemic inflammatory response in a human patient with an acute illness. In some aspects, the acute illness can be burns, major surgery, sepsis, autoimmune disorders, vasculitis, thromboembolism, trauma, and acute pancreatitis. Disclosed herein are methods of preventing systemic inflammatory response in a human patient with acute pancreatitis. Disclosed herein are methods of preventing systemic inflammatory response or multi-organ failure in a human patient with an acute illness. In some aspects, the acute illness can be burns, major surgery, sepsis, autoimmune disorders, vasculitis, thromboembolism, trauma, and acute pancreatitis. Disclosed herein are methods of preventing systemic inflammatory response or multi-organ failure in a human patient with acute pancreatitis. The method can comprise: (a) identifying a human patient in need of treatment; and (b) administering to the human patient a therapeutically effective amount of a pharmaceutical composition comprising glucagon-like peptide (GLP)-2 or a GLP-2 analog; and a pharmaceutically acceptable carrier.

Also, disclosed herein are methods of preventing or reducing endotoxin entry in subject's portal vein. In some aspects, preventing or reducing entry into the portal vein thereby reduces hepatic inflammation, which thereby reduces the generation of systemic pro-inflammatory cytokines that in turn reduces inflammation in a subject's liver, lungs, kidneys, brain, hematopoietic system, gastrointestinal system, blood coagulation system, vascular system, and other systems. Also, disclosed herein are methods of preventing or reducing endotoxin entry in subject's lung or liver. Also, disclosed herein are methods of preventing or reducing endotoxin entry in subject's lung, liver, kidney, brain, hematopoietic system, gastrointestinal system, blood coagulation system, vascular system, and other systems. The method can comprise: (a) identifying a human patient in need of treatment; and (b) administering to the human patient a therapeutically effective amount of a pharmaceutical composition comprising glucagon-like peptide (GLP)-2 or a GLP-2 analog; and a pharmaceutically acceptable carrier. In some aspects, endotoxin entry into the portal vein can be reduced or prevented. In some aspects, endotoxin entry into the portal vein can be reduced or prevented by administering to a subject a GLP-2 or GLP-analog. In some aspects, endotoxin entry into the portal vein can be reduced or prevented by administering to a subject a GLP-2 or GLP-analog 12 hours before exposure to an inflammatory stimulus or before the induction of systemic inflammation. In some aspects, the GLP-2 or GLP-2 analogs can be administered at 1 hour, 2 hours, 3 hours, 4 hours, 5 hours, 6 hours, 7 hours, 8 hours, 9 hours, 10 hours, 11 hours or 12 hours or any time in between before exposure to an inflammatory stimulus or before the induction of systemic inflammation. In some aspects, the GLP-2 or GLP-2 analogs can be administered at 1 s, 5 s, 10 s, 15 s, 20 s, 25 s, 30 s, 35 s, 40 s, 45 s, 50 s, 55 s, 60 s, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes or any time in between before exposure to an inflammatory stimulus or before the induction of systemic inflammation. In some aspects, endotoxin entry into the portal vein can be reduced or prevented by administering to a subject a GLP-2 or GLP-analog 1 s to 12 hours after exposure to an inflammatory stimulus or after the induction of systemic inflammation. In some aspects, the GLP-2 or GLP-2 analogs can be administered at 1 hour, 2 hours, 3 hours, 4 hours, 5 hours, 6 hours, 7 hours, 8 hours, 9 hours, 10 hours, 11 hours or 12 hours or any time in between after exposure to an inflammatory stimulus or after the induction of systemic inflammation. In some aspects, the GLP-2 or GLP-2 analogs can be administered at 1 s, 5 s, 10 s, 15 s, 20 s, 25 s, 30 s, 35 s, 40 s, 45 s, 50 s, 55 s, 60 s, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes or any time in between after exposure to an inflammatory stimulus or after the induction of systemic inflammation. In some aspects, endotoxin entry into the portal vein can be reduced or prevented in the patient's liver of lungs. In some aspects, endotoxin entry into the patient's liver or lungs can be reduced or prevented thereby reducing reducing expression or preventing an increase in the expression of one or more cytokines. In some aspects, the one or more cytokines can be tumor necrosis factor alpha, interleukin-1β or interleukin-6, or any other pro-inflammatory cytokine. In some aspects, the one or more cytokines can be systemically expressed.

Also, disclosed herein, are methods of ameliorating one or more symptoms of acute pancreatitis or inflammatory illness in a subject. The method can comprise: (a) identifying a subject in need thereof; and (b) administering to the subject a therapeutically effective amount of glucagon-like peptide (GLP)-2 or a GLP-2 analog.

In some aspects, in any of the methods disclosed herein, the patient or subject in need thereof can have or be suspected of having acute illnesses including but not limited to burns, major surgery, sepsis, autoimmune disorders, vasculitis, thromboembolism, trauma, and acute pancreatitis. In some aspects, in any of the methods disclosed herein, the patient or subject in need thereof can have or be suspected of having acute pancreatitis.

GLP-2 or a GLP-2 analog as described herein can be administered in conjunction with other therapeutic modalities to a subject in need of therapy. GLP-2 or a GLP-2 analog can be administered prior to, simultaneously with or after treatment with other agents or regimes, for example, intravenous rehydration, antibiotics, vasopressors, mechanical ventilation, and other life-support measures or a combination thereof. In some aspects, the method can further comprise administration of intravenous rehydration, antibiotics, vasopressors, mechanical ventilation, and other life-support measures or combination thereof.

In some aspects, the GLP-2 analog can be teduglutide. In some aspects, the GLP-2 analog can be a GLP-2 analog that can be metabolized by hydrolytic enzymes such as but not limited to dipeptidyl peptidase 4. In some aspects, the GLP-2 analog can be a short-acting GLP-2 analog. The term “short acting” can mean producing an effect within seconds to minutes of administration and lasting less than 1 hr.

In some aspects, a therapeutically effective amount of a pharmaceutical composition comprising glucagon-like peptide (GLP)-2 or a GLP-2 analog and a pharmaceutically acceptable carrier can be administered to the subject. In some aspects, the administration of GLP-2 or a GLP-2 analog can reduce one or more symptoms of an acute illness. In some aspects, the acute illness can be burns, major surgery, sepsis, autoimmune disorders, vasculitis, thromboembolism, trauma, and acute pancreatitis. In some aspects, the administration of GLP-2 or a GLP-2 analog can reduce one or more symptoms of acute pancreatitis. In some aspects, the subject can be a human. In some aspects, one or more of the symptoms of an acute illness can be reduced over a period of at least 1 hour, at least 2 hours, at least 3 hours, at least 4 hours or more, at least 1 day, at least 2 days, at least 4 days, at least 5 days, at least 6 days, at least 7 days, or more. In some aspects, the subject can be a human. In some aspects, one or more of the symptoms of an acute pancreatitis can be reduced over a period of at least 1 hour, at least 2 hours, at least 3 hours, at least 4 hours or more, at least 1 day, at least 2 days, at least 4 days, at least 5 days, at least 6 days, at least 7 days, or more. In some aspects, the one or more symptoms of an acute illness can be detected at the onset of abnormal body temperature, leukocytosis, abnormal blood pressure, elevated respiratory rate or at the signs of onset of acute illness such as abdominal pain, or following trauma, surgery, burns, or other physiological insults. In some aspects, the one or more symptoms of acute pancreatitis can be selected from acute onset of abdominal pain, nausea, vomiting, dyspnea and fever.

The pharmaceutical compositions described herein can be formulated to include a therapeutically effective amount of GLP-2 or a GLP-2 analog. In some aspects, GLP-2 or a GLP-2 analog can be contained within a pharmaceutical formulation. In some aspects, the pharmaceutical formulation can be a unit dosage formulation. In some aspects, GLP-2 or a GLP-2 analog can administered on an as-needed basis. In some aspects, GLP-2 or a GLP-2 analog can administered for a predetermined time period.

Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to one or more side effects associated with any one of the acute illnesses described herein including but not limited to burns, major surgery, sepsis, autoimmune disorders, vasculitis, thromboembolism, trauma, and acute pancreatitis, extrapancreatic organ inflammation, systemic inflammatory response syndrome (SIRS) and/or multi-organ failure. In some aspects, the acute illness can be acute pancreatitis.

The pharmaceutical compositions described herein can be administered to the subject (e.g., a human patient) in an amount sufficient to delay, reduce, prevent, or reverse the onset or duration of one or more symptoms or signs associated with any one of the acute illnesses disclosed herein including but not limited to burns, major surgery, sepsis, autoimmune disorders, vasculitis, thromboembolism, trauma, and acute pancreatitis, extrapancreatic organ inflammation, SIRS and/or multi-organ failure. In some aspects, the acute illness can be acute pancreatitis. Accordingly, in some aspects, the patient can be a human patient. In therapeutic applications, compositions are administered to a subject (e.g., a human patient) already expressing or diagnosed with any of the acute illnesses disclosed herein including but not limited to burns, major surgery, sepsis, autoimmune disorders, vasculitis, thromboembolism, trauma, and acute pancreatitis, an acute inflammatory disorder associated with SIRS, systemic organ inflammation, extranpancreatic organ inflammation and/or multi-organ failure in an amount sufficient to at least partially improve a sign or symptom or to inhibit the progression of (and preferably arrest) the symptoms of the condition, its complications, and consequences. An amount adequate to accomplish this is defined as a therapeutically effective amount. A therapeutically effective amount of a pharmaceutical composition can be an amount that achieves a cure or reverses one or more signs or symptoms of any of the acute illnesses disclosed herein including but not limited to burns, major surgery, sepsis, autoimmune disorders, vasculitis, thromboembolism, trauma, and acute pancreatitis, an acute inflammatory disorder associated with SIRS, systemic organ inflammation, extrapancreatic organ inflammation and/or multi-organ failure, but that outcome is only one among several that can be achieved. As noted, a therapeutically effect amount includes amounts that provide a treatment in which the onset, progression or expression of one or more symptoms or signs associated with any of the acute illnesses disclosed herein including but not limited to burns, major surgery, sepsis, autoimmune disorders, vasculitis, thromboembolism, trauma, and acute pancreatitis, organ inflammation, extrapancreatic organ inflammation, SIRS and/or multi-organ failure is delayed, hindered, or prevented, or the one or more symptoms or signs associated with any of the acute illnesses disclosed herein including but not limited to burns, major surgery, sepsis, autoimmune disorders, vasculitis, thromboembolism, trauma, and acute pancreatitis, systemic organ inflammation, extrapancreatic organ inflammation, SIRS and/or multi-organ failure is reduced, ameliorated or reversed. One or more of the symptoms can be less severe. Recovery can be accelerated in an individual who has been treated.

Amounts effective for this use can depend on the severity of the signs or symptoms associated with any of the acute illnesses disclosed herein including but not limited to burns, major surgery, sepsis, autoimmune disorders, vasculitis, thromboembolism, trauma, and acute pancreatitis, systemic organ inflammation, extrapancreatic organ inflammation, SIRS and/or multi-organ failure and the weight and general state and health of the subject, but generally range from about 0.05 to 1 mg/kg/day of an equivalent amount of the GLP-2 or a GLP analog per day per subject.

The total effective amount of GLP-2 or a GLP-2 analog as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The therapeutically effective amount or dosage of the GLP-2 or a GLP-2 analog used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage concentrations can be adjusted using standard empirical routes for optimization. The particular dosage of a pharmaceutical composition to be administered to the patient will depend on a variety of considerations (e.g., the severity of side effects of GLP-2 and GLP-2 analogs; the severity of the disease, disorder or condition), the age and physical characteristics of the subject and other considerations known to those of ordinary skill in the art. Dosages of GLP-2 or a GLP-2 analog can be in the range of 0.05 mg to 1 mg per kilogram of the subject's body weight. In some aspects, the dosage of GLP-2 or a GLP analog can be 0.05, 0.1, 0.15, 0.2 0.3, 0.35, 0.4, 0.45, 0.5, 0.55, 0.6, 0.65, 0.7, 0.75, 0.8, 0.85, 0.9, 0.95 or 1 mg/kg/day total. In some aspects, the dosage of GLP-2 or a GLP-2 analog can be 0.05 to 1 mg/kg/day. In some aspects, GLP-2 or a GLP -2 analog can be administered intravenously. In some aspects, GLP-2 or a GLP -2 analog can be administered through intravenous infusion. In some aspects, the intravenous infusion can be intermittent or continuous. In some aspects, the continuous intravenous infusion can occur over a period of about 24 hours. In some aspects, the continuous intravenous infusion can occur over a period of at least 24 hours to about 7 days. In some aspects, the intermittent intravenous infusion can occur up to 12 times per day. In some aspects, GLP-2 or a GLP analog can be administered subcutaneously. In some aspects, the pharmaceutical compositions disclosed herein, formulated for subcutaneous injection can be administered once or twice per day. In some aspects, the therapeutically effective amount of GLP-2 or a GLP-2 analog can be between 0.05 to 1 mg/kg/day of body weight or any amount in between.

### PHARMACEUTICAL COMPOSITIONS

As disclosed herein, are pharmaceutical compositions, comprising GLP-2 or a GLP-2 analog and a pharmaceutically acceptable carrier described herein. In some aspects, GLP-2 or a GLP-2 analog can be formulated for intravenous administration. In some aspects, the compositions disclosed herein can be formulated for intravenous administration for intermittent or continuous intravenous infusion. In some aspects, the compositions disclosed herein can be formulated for continuous intravenous infusion in a rapid release form. In some aspects, GLP-2 or a GLP-2 analog can be formulated for subcutaneous administration. In some aspects, GLP-2 or a GLP-2 analog can be formulated for slow release. In some aspects, the compositions disclosed herein can be formulated for subcutaneous administration in a slow release form. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

The compositions described herein can be formulated with a carrier that can be pharmaceutically acceptable and that can be appropriate for delivering the peptide by the desired route of administration. Suitable pharmaceutically acceptable carriers can be those that are typically used with peptide-based drugs, such as diluents, excipients and the like. Reference can be made to “Remington's Pharmaceutical Sciences” by E. W. Martin, which is herein incorporated by reference, for guidance on drug formulations generally. In some aspects, the carrier can be selected based on its ability to solubilize and stabilize the peptide in solution.

Further, the carrier can be selected based its ability to permit the release of the peptide into circulation after, for example, injection.

In some aspects, the compositions can be formulated for administration by infusion. In some aspects, the compositions can be formulated for administration by injection (e.g., subcutaneously, intramuscularly or intravenously) and can be used as aqueous solutions in sterile and pyrogen-free form and optionally buffered to physiologically tolerable pH, e.g., a slightly acidic or physiological pH. The compositions can be administered in a vehicle such as distilled water or in saline, phosphate buffered saline or 5% dextrose solution. Incorporating a solubility enhancer, such as acetic acid, can enhance water solubility of the compositions described herein. The aqueous carrier or vehicle can be supplemented for use as injectables with an amount of gelatin that can serve to depot the GLP-2 or GLP-2 analog at or near the site of injection, for its slow release to the desired site of action. Concentrations of gelatin effective to achieve the depot effect can be in the range of 10-20%. Alternative gelling agents, such as hyaluronic acid, can also be useful as depoting agents.

In some aspects, GLP-2 or a GLP-2 analog can be formulated as a slow release implantation device for extended and sustained administration. Examples of such sustained release formulations include but not limited to composites of biocompatible polymers, such as poly(lactic acid), poly(lactic-co-glycolic acid), methylcellulose, hyaluronic acid, collagen, and the like. Liposomes can also be used to provide for the sustained release of GLP-2 or GLP-2 analogs. Implantable osmotic minipumps can also be used for sustained release. Sustained release formulations can provide a high local concentration of GLP-2 or GLP-2 analogs. In some aspects, the compositions described herein can be formulated for sustained release.

The compositions disclosed herein can be used in the form of a sterile-filled vial or ampoule that can contain a desired amount of the peptide in either unit dose or multi-dose amounts. The vial or ampoule can contain the GLP-2 or GLP-2 analog and the desired carrier as an administration-ready formulation. Alternatively, the vial or ampoule can contain the GLP-2 or GLP-2 analog peptide in a form, such as a lyophilized form, suitable for reconstitution in a suitable carrier, such as phosphate-buffered saline.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, intraportal, transmucosal (e.g., intranasal, intravaginal, or rectal), or transdermal (e.g., topical) administration. Aerosol inhalation can also be used. Thus, compositions can be prepared for parenteral administration that includes GLP-2 or a GLP-2 analog dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

### ARTICLES OF MANUFACTURE

The composition described herein can be packaged in a suitable container labeled, for example, for use to suppress or prevent extrapancreatic organ inflammation or systemic organ inflammation in subjects with an acute illness including but not limited to burns, major surgery, sepsis, autoimmune disorders, vasculitis, thromboembolism, trauma, and acute pancreatitis, acute inflammatory disorders associated with SIRS and/or MOF; to prevent the development of multi-organ failure in subjects with an acute illness including but not limited to burns, major surgery, sepsis, autoimmune disorders, vasculitis, thromboembolism, trauma, and acute pancreatitis; to prevent or reduce endotoxin entry in a subject's lung or liver or portal vein; and/or to ameliorate one or more symptoms of an acute illness including but not limited to burns, major surgery, sepsis, autoimmune disorders, vasculitis, thromboembolism, trauma, and acute pancreatitis. Accordingly, packaged products (e.g., sterile containers containing the composition described herein and packaged for storage, shipment, or sale at concentrated or ready-to-use concentrations) and kits, including at least GLP-2 or a GLP-2 analog as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compounds can be ready for administration (e.g., present in dose-appropriate units), and may include a pharmaceutically acceptable adjuvant, carrier or other diluent. Alternatively, the compounds can be provided in a concentrated form with a diluent and instructions for dilution.

### EXAMPLES

### Example 1: Teduglutide Ameliorates Extra-Pancreatic Inflammation Through Reduction of Portal Venous LPS Levels in a Murine Model of Pancreatitis

Background. Glucagon-like peptide-2 (GLP-2), a peptide hormone derived from enteroendocrine L cells present in small and large intestine, is important for maintenance of intestinal villi through regulation of proliferation and differentiation. The stable GLP-2 analog teduglutide (TDG) is approved for patients with severe malabsorption who are dependent on parenteral nutrition due to its intestinotrophic effects. GLP-2 also has acute effects on gastrointestinal motility, secretion, and barrier function independent of its pro-proliferative effects. Since decreased barrier function is associated with severe inflammation, increased circulating lipopolysaccharides (LPS), and often-fatal consequences such as multiple organ failure (MOF), it was tested whether TDG, by strengthening the gut barrier and limiting LPS uptake from its reservoir in the gut lumen to the portal vein (PV) could reduce the severity of MOF in an experimental model of pancreatitis. Pancreatitis was chosen since it is a common antecedent of clinical MOF and since multiple organ inflammation develops in its experimental models as well.

LPS entry from the small intestine suggests that elevated PV LPS concentrations may substantially contribute to LPS-related organ damage, such as liver, lung, or renal injury during serious illness, and that exogenous GLP-2 may impede LPS entry from the gut lumen to the PV, decreasing liver inflammation which in turn decreases the systemic inflammatory response. Severe acute pancreatitis has a mortality rate >40% mostly due to the development of multiple organ failure 2-3 days after symptom development that has been attributed to systemic endotoxemia. Therefore, it was tested whether TDG treatment may reduce multiple organ injury following acute pancreatitis by reduction of LPS entry from the intestine to the PV.

Methods. Pancreatitis was induced in C57BL/6 male and female mice at 8-10 weeks of age by multiple injections of cerulein (50 μg/kg, ip, hourly, 8 times). Samples (e.g., PV and abdominal arterial blood) were collected 0, 18, and 24 hrs after the final injection of cerulein. Tissues were removed and stored for histology and real-time polymerase chain reaction (PCR)-based expression analysis. TDG, (500 μg/kg, ip) was given 0 and 18 hrs after the final injection of cerulein, followed by sampling 24 hrs after the final injection of cerulein. Proinflammatory cytokines was assessed by real-time PCR; PV LPS levels were measured using the limulus amebocyte lysate (LAL) test.

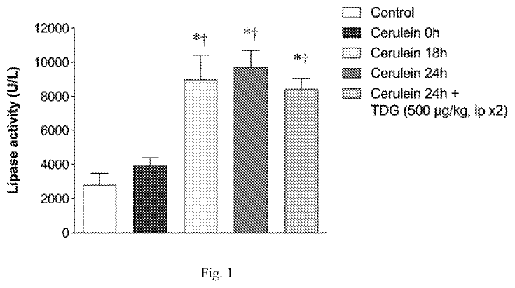

Results. Cerulein treatment successfully induced pancreatitis, confirmed by the increased plasma lipase activity (FIG. 1). Compared with the controls, systemic expression of the proinflammatory cytokines tumor necrosis factor (TNF)-α, interleukin (IL)-1 β, IL-6, and IFNγ were increased in the ileum, liver and lung (ileum<liver<lung), indicating multiple organ inflammation, 24 hrs after the final injection of cerulein.

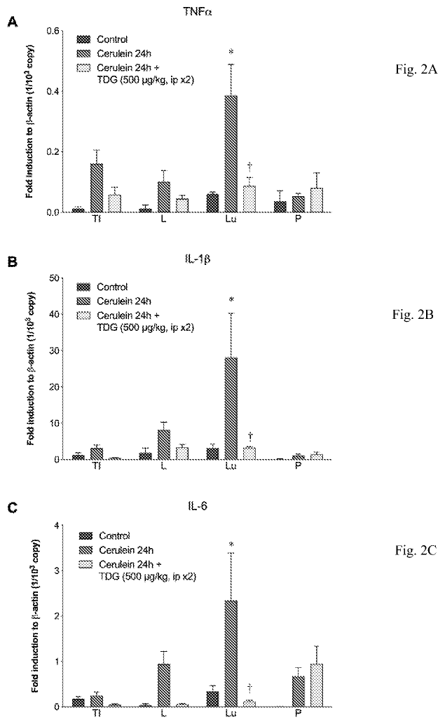

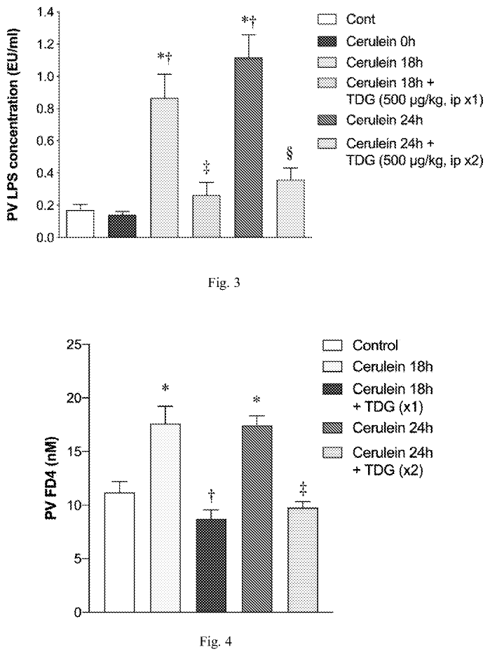

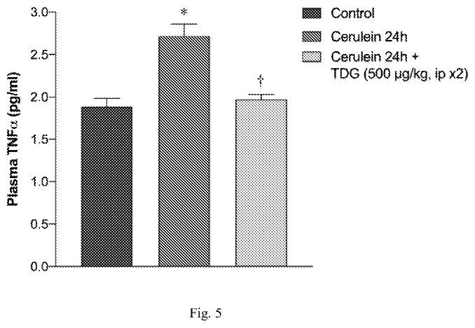

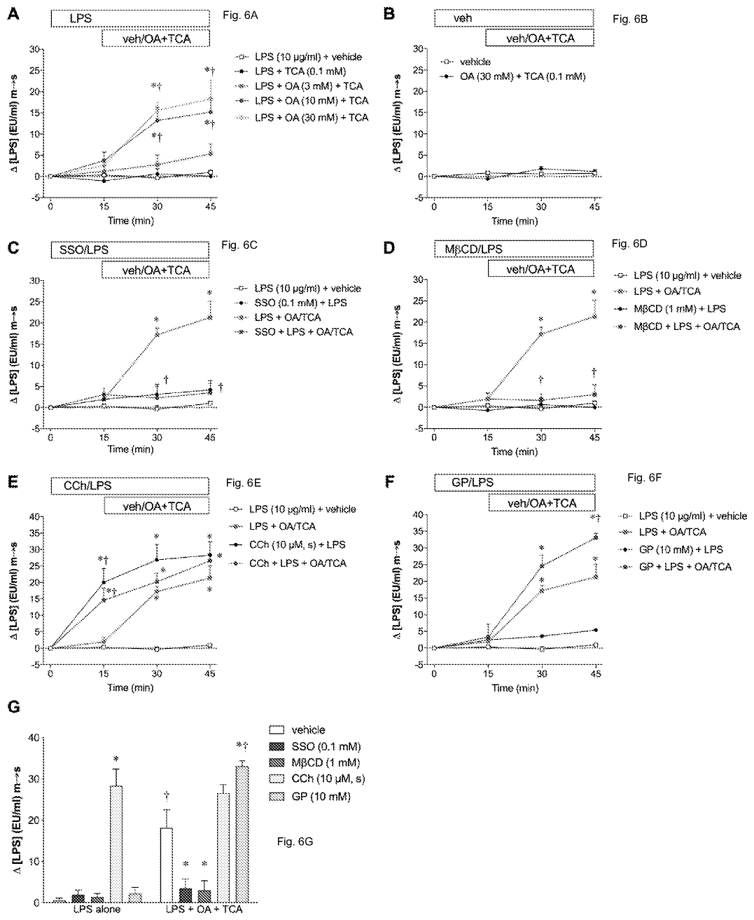

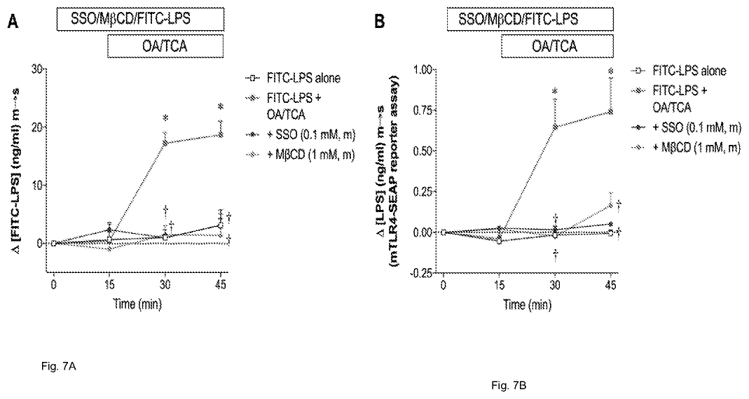

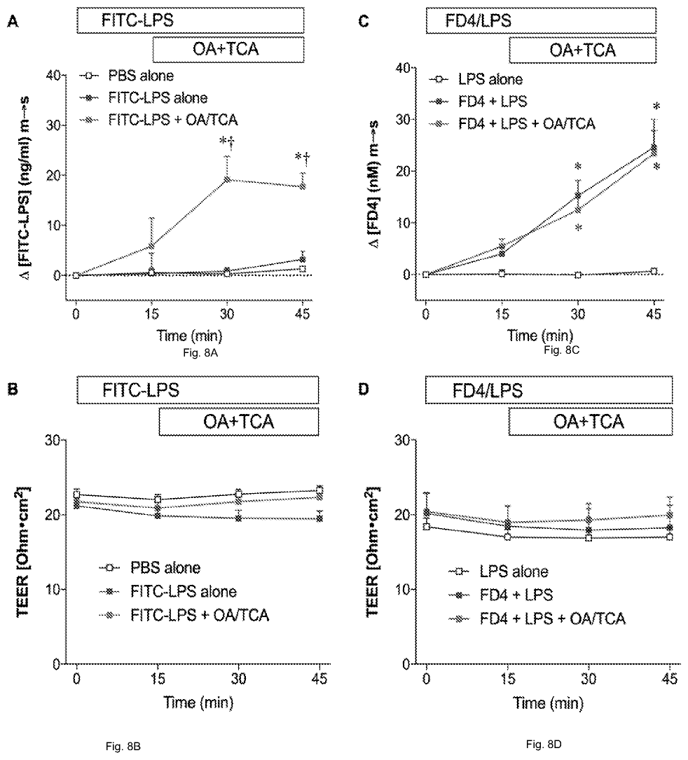

TDG treatment significantly reduced inflammatory cytokine expression in the lung (FIG. 2), whereas TDG had no effect on elevated lipase levels (FIG. 1) and increased expression of IL-1β and IL-6 in the pancreas following cerulein injection (FIG. 2). PV LPS levels (FIG. 3) were increased 18 and 24 hrs after cerulein treatment, but not at 0 hr, accompanied by increased uptake of the paracellular permeability marker FITC-dextran 4000 (FD4) from the small intestine 18 or 24 hrs after cerulein treatment (FIG. 4), suggesting that LPS entry occurs after a pancreatitis-associated intestinal permeability increase. TDG treatment significantly reduced PV LPS levels (FIG. 3) and PV FD4 levels (FIGS. 4) 18 and 24 hrs after cerulein treatment. Plasma TNFα levels, indicative of systemic inflammation, were increased 24 hrs after cerulein treatment, reversed by TDG treatment (FIG. 5). Histology confirmed mild inflammation in the lung and edematous changes in the pancreas 24 hrs after cerulein treatment. TDG treatment reduced the lung inflammation but had no effect on the histological changes in the pancreas (FIG. 6).

Discussion. The effect of TDG in an experimental model of acute pancreatitis was tested. TDG (500 μg/kg) was given at 10× the recommended daily clinical dose at 0 and 18 hrs after the final dose of cerulein. Remarkably, although TDG had minimal effects on pancreatic inflammation, it reversed lung inflammatory markers and PV LPS concentrations to near control concentrations. These results suggest that pancreatitis-associated lung injury is induced by intestinal LPS entry into the PV, accompanied by increased small intestinal permeability to FD4, and that TDG reduces systemic and extrapancreatic organ inflammation through inhibition of LPS entry into the PV through the compromised gut barrier. TDG may be a novel therapeutic option for pancreatitis-associated MOF through its prevention of LPS entry from the intestines to the PV.

### REFERENCES

Burgmaier M, Liberman A, Möllmann J, Kahles F, Reith S, Lebherz C, Marx N, Lehrke M. Glucagon-like peptide-1 (GLP-1) and its split products GLP-1(9-37) and GLP-1(28-37) stabilize atherosclerotic lesions in apoe ⁻/⁻ mice. Atherosclerosis. 2013 December; 231(2):427-35.

Thulesen J, Knudsen L B, Hartmann B, Hastrup S, Kissow H, Jeppesen P B, Ørskov C, Hoist J J, Poulsen S S. The truncated metabolite GLP-2 (3-33) interacts with the GLP-2 receptor as a partial agonist. Regul Pept. 2002 Jan. 15; 103(1):9-15. PubMed PMID: 11738243.

Gallwitz B, Witt M, Morys-Wortmann C, Fölsch U R, Schmidt W E. GLP-1/GIP chimeric peptides define the structural requirements for specific ligand-receptor interaction of GLP-1. Regul Pept. 1996 May 7;63(1):17-22. PubMed PMID: 8795084.

Kaukonen K M, Bailey M, Pilcher D, Cooper D J, Bellomo R. Systemic inflammatory response syndrome criteria in defining severe sepsis. N Engl J Med. 2015 Apr 23; 372(17):1629-38.

### Example 2: Lipopolysaccharides (LPS) Transport During Fat Absorption in Rodent Small Intestine

Abstract. The mechanisms and the effect of exogenous glucagon-like peptide-2 (GLP-2) on lipopolysaccharides (LPS) transport in rodent small intestine was investigated. Transmucosal LPS transport was measured in Ussing-chambered rat jejunal mucosa. In anesthetized rats, the appearance of FITC-LPS into the portal vein (PV) and the mesenteric lymph was simultaneously monitored after intraduodenal perfusion of FITC-LPS with oleic acid and taurocholate (OA/TCA). In vitro, luminally-applied LPS rapidly appeared in the serosal solution limited with luminal OA/TCA present. OA/TCA-induced LPS transport was inhibited by luminal pretreatment with the lipid raft inhibitor methyl-β-cyclodextrin (MβCD) and the CD36 inhibitor sulfosuccinimidyl oleate (SSO), or by serosally-applied GLP-2 with dipeptidyl peptidase-4 inhibition. In vivo, perfusion of FITC-LPS with OA/TCA rapidly increased FITC-LPS appearance into the PV, followed by a gradual increase of FITC-LPS into the lymph. Rapid PV transport was inhibited by the addition of MβCD or by SSO, whereas transport into the lymph was inhibited by chylomicron synthesis inhibition. IV injection of the stable GLP-2 analog teduglutide acutely inhibited PV FITC-LPS transport into the PV, yet accelerated FITC-LPS transport into the lymph via L-NAME- and PG97-269-sensitive mechanisms. FITC-LPS recovery was ˜60% in PV and ˜1% in lymph. In vivo two-photon confocal microscopy in mouse jejunum confirmed intracellular FITC-LPS uptake limited to luminal OA/TCA present. Conclusions: Luminal LPS may cross the small intestinal barrier physiologically during fat absorption via lipid raft- and CD36-mediated mechanisms, followed by predominant transport into the PV. Inhibition of LPS uptake into the PV by GLP-2 may mitigate its pro-inflammatory effects.

Introduction. Lipopolysaccharides (LPS; endotoxin), a lipophilic pathogen-associated molecular pattern (PAMP) derived from Gram-negative bacteria that is resistant to heat, low pH, and proteases (16), is a potent human toxin. Although LPS is abundant in the environment and is present in foods such as ice cream, yogurt, and meat (16), it is also present in saliva derived from oral flora (41). LPS is a ˜10-15 kDa molecule, consisting of polysaccharide chains and lipid A, the latter having its pyrogenic endotoxic activity. Lipid A has two phosphorylation sites in the disaccharide backbone that have ester- and amine-linked fatty acids. Phosphorylated lipid A is biologically active; subsequent binding to Toll-like receptor 4 (TLR4) (55), initiates an intense inflammatory cascade eventuating in fever, tachycardia, and hypotension that provides the basis for referring to endotoxins as “pyrogens”. Injection of a 2 ng/kg dose of LPS into normal humans reproducibly induces fever, tachycardia and tachypnea (20) whereas higher doses induce the circulatory collapse and respiratory failure associated with septic shock and multiple organ failure (67). The major reservoir of LPS is the gut lumen, since it contains 10³-10¹¹ bacteria/ml of which ˜50% are Gram-negative, translating to an LPS concentration of ˜0.01-1,000,000 ng/ml assuming 25 fg LPS/bacterial cell, compared with a circulating plasma LPS concentration of 0.03 ng/ml, a ˜1:3 lumen:plasma gradient in the proximal foregut and a ˜3×10⁷:1 gradient in the colon, consistent with the presence of multiple LPS detoxifying mechanisms and barriers to intestinal LPS uptake. Defenses include intestinal alkaline phosphatase (IAP) an enzyme with high activity in the brush border membranes of the duodenum and jejunum (4) that detoxifies LPS through dephosphorylation of the lipid A of LPS (24, 40), preventing its binding to TLR4 (71). Due to its molecular size and detoxification of LPS by IAP, some believe that luminal LPS may be absorbed by the foregut intestinal epithelium or enter into the body by the paracellular pathway in the presence of mucosal injury. Increased serum LPS concentrations are observed in humans and in experimental animals following consumption of a high-fat diet, linking such diets with increased LPS entry from small intestine into the circulation (8). Chronic mild elevations of circulating LPS are termed “metabolic endotoxemia” given their association with chronic low-grade inflammation that characterizes the metabolic syndrome and also diseases associated with chronic low-grade inflammation such as Alzheimer's disease (7, 75).

A high-fat meal acutely increases circulating LPS levels in human healthy volunteers at 30 min (18), suggesting that dietary lipid facilitates LPS absorption. Gavage of a long-chain triglyceride triolein solution in mice increases circulating LPS levels, compared to a short-chain triglyceride tributyrate control solution (23). LPS is incorporated into chylomicron remnants, a process inhibited by the chylomicron synthesis inhibitor Pluronic-81 (PL81) (23), suggesting that LPS is absorbed by the same mechanism as are long-chain fatty acids (LCFAs). Absorbed LPS is primarily cleared from the circulation by the liver. Hepatic macrophages (Kupffer cells) appear to be the principal cells involved in the clearance of LPS (19); intraportal LPS is rapidly taken up by hepatocytes and excreted into the bile, peaking within 20 min (45). Furthermore, chylomicrons accelerate LPS clearance from the plasma by increasing the hepatic uptake of chylomicron-incorporated LPS followed by its increased excretion into the bile (58). Both chylomicrons and bile salts reversibly inhibit LPS pyrogenic activity (15, 58, 60). These results suggest that chylomicron-mediated LPS uptake is linked to multiple protective mechanisms that diminish LPS toxicity, preventing hepatic inflammation following Kupffer cell activation by unbound LPS.

Biological processes in the small intestinal lumen mediate lipid absorption. Luminal triglycerides (TG) combine with bile acids, generating micelles that solubilize TG, which are then de-esterified to LCFAs and mono-acyl glycerol (MAG) by pancreatic lipase. Digested luminal fat, LCFAs, and MAG are primarily absorbed by jejunal enterocytes via active and passive pathways. LCFAs are partially absorbed via cluster-of-differentiation (CD) 36-mediated and fatty acid transport protein (FATP)-mediated transport, followed by passive diffusion into the enterocytes (65). Genetic deletion of CD36 or FATP isoforms had no effect on fat absorption in vivo (25, 64), whereas CD36 and FATP are important for LCFA transport in vitro (50, 65), suggesting that these active transport molecules likely comprise a high affinity, low capacity transport system. Lipid raft-mediated endocytosis also contributes to LCFA absorption, since CD36 localizes to cholesterol-rich lipid rafts (14). Absorbed LCFA and MAG are re-esterified to TG, and subsequently combined with apolipoprotein B-48, forming nascent chylomicrons that are released by exocytosis across the enterocyte basolateral membrane into the central lacteals and villous lymphatic capillaries, draining into the mesenteric lymph ducts, followed by entry into the systemic circulation via the thoracic duct (69). Since the principal origin of circulating LPS is the gut lumen, the mechanisms of LPS transport across the gut mucosa was studied, testing whether transport occurs via canonical lipid uptake pathways.

Glucagon-like peptide-2 (GLP-2) is an intestinotrophic hormone released from enteroendocrine L cells (12). Chronic treatment with GLP-2 prevents the appearance of LPS in the circulation (8), attributed mostly to its pro-proliferative effects on the intestinal epithelium. Since GLP-2 reduces intestinal paracellular permeability (8), but acutely enhances fat absorption (33), it was also tested whether GLP-2 acutely affects LPS transport during fat absorption.

Materials and methods. Animals. Male Sprague-Dawley rats weighing 200-250 g and C57BL/6 mice weighing 20-25 g (Harlan, San Diego, Calif., USA) were fed a pellet diet and water ad libitum. Rats were fasted overnight with free access to water before the experiments, whereas mice were fasted for 3 hrs before the experiments in order to empty the stomach. Animals were euthanized by terminal exsanguination under deep isoflurane anesthesia, followed by thoracotomy.

Chemicals. Teduglutide (TDG, Shire Pharmaceuticals USA, Lexington, Mass.) was provided by the Pharmacy Service of the West Los Angeles Va. Rat GLP-2, NVP-728, NVP-AEW-541 and PD153035 were purchased from Tocris Bioscience (Ellisville, Mo., USA). The vasoactive intestinal peptide (VIP)/pituitary adenylate cyclase-activating peptide (PACAP) receptor 1 (VPAC1) antagonist PG97-269; [Ac-His¹, D-Phe², Lys¹⁵, Arg¹⁶, Leu²⁷]-VIP(1-7)-GRF(8-27) (SEQ ID NO: 41) (26) was purchased from Phoenix Pharmaceuticals (Burlingame, Calif., USA) or was synthesized using solid-phase methodology according to the Fmoc-strategy using an automated peptide synthesizer (Model Pioneer, Thermo Fisher Scientific, Waltham, Mass., USA). The crude peptide was purified using reverse-phase high performance liquid chromatography (HPLC: Delta 600 HPLC System, Waters, Mass., USA) on a column of Develosil ODS-HG-5 (2×25 cm, Nomura Chemical Co., Ltd, Seto, Japan). The purity of each peptide was confirmed by analytical HPLC and matrix assisted laser desorption/ionization time of flight and mass spectrometry (MALDI-TOF MS) analysis. seminaphthorhodafluor (SNARF™)-5F 5-(and-6)-carboxylic acid (SNARF-5F) was purchased from Molecular Probes (Eugene, Oreg., USA). Oleic acid (OA), taurocholic acid (TCA), LPS (from E. coli 055:B5), fluorescein isothiocyanate (FITC)-conjugated LPS (FITC-LPS; from E. coli 055:B5), FITC-dextran 4000 (FD4), carbachol (CCh), glycerol phosphate (GP), methyl-β-cyclodextrin (MβCD), sulfosuccinimidyl oleate (SSO), PL81, Nω-nitro-L-arginine methyl ester (L-NAME), indomethacin (IND), and other chemicals were purchased from Sigma Chemical (St. Louis, Mo., USA). IND was dissolved in ethanol. OA was added in TCA-containing phosphate buffer saline (PBS, pH 7.4), followed by vigorous vortexing just prior to performing the experiments. The other chemicals were dissolved in distilled water in order to make a stock solution.

Short-circuit current measurements in Ussing chambered preparations. Mucosa-submucosa preparations were prepared from the mid-jejunum (˜20 cm from the pyloric ring) by muscle stripping using fine forceps under a stereomicroscope. Two or four preparations were prepared from one animal. The tissue was then mounted between two hemi-sliders with an aperture=0.3 cm² (Physiologic Instruments, San Diego, Calif., USA). Chambers were bathed with serosal and luminal bathing solutions in a volume of 4 ml each, maintained at 37° C. using a water recirculating heating system. The serosal Krebs-Ringer solution contained (in mM) NaCl, 117; KCl, 4.7; MgCl₂, 1.2; NaH₂PO₄, 1.2; CaCl₂, 2.5; NaHCO₃, 25; glucose, 11; and bubbled with 95% O₂-5% CO₂ to maintain pH at 7.4. The luminal bathing solution contained NaCl, 136; KCl, 2.6; CaCl₂, 1.8; HEPES, 10 (pH 7.0); mannitol, 11; and bubbled with 100% O₂. The tissues were short-circuited by a voltage clamp (VCC MC6, Physiologic Instruments) at zero potential difference with automatic compensation for solution resistance. Short-circuit current (I_{sc}) was continuously measured with tissue conductance (Gₜ) and TEER (Ω·cm²) determined every 20 sec in order to assess the tissue integrity during the LPS and lipid exposure. The current was recorded by the DataQ system (Physiologic Instruments). IND (10 μM) was added to the serosal bath to eliminate the effects of endogenous prostaglandin production. The tissues were stabilized for ˜30 min before the addition of test chemicals.

LPS movement from mucosal to serosal solution. LPS movement m-to-s was assessed using a LAL test kit (Pierce Chromogenic Endotoxin Quant Kit, Pierce Biotechnology, Rockford, Ill., USA), which eliminates non-specific detection of (1,3)-β-D-glucan, then reduces false-positive LPS measurements. After tissue stabilization, time was set as t=0 min. Fifty μl of the serosal solution was collected at 0 min as a blank sample, followed by collection at t=15, 30 and 45 min. At t=0 min, LPS (10 μg/ml) was added into the mucosal solution. Vehicle (PBS alone), TCA alone (0.1 mM) in PBS or OA (3, 10, 30 mM) with TCA (0.1 mM) in PBS was added into the mucosal solution at t=15 min. The tissues were pretreated with drugs 10 or 15 min before the addition of LPS (i.e. at t=−10 or −15 min). The collected serosal solutions were kept at −80° C. until use. LPS content in the serosal solutions was measured with the LAL test according to the manufacturer's protocol. The value of background at t=0 min was subtracted and the serosal LPS appearance was expressed as Δ[LPS] (EU/ml). In some experiments, FITC-LPS (10 μg/ml) was used instead of LPS. Serosal FITC-LPS content was assessed by FITC fluorescence intensity measurement using a multi-mode microplate reader (Synergy-2, BioTek Instruments, Inc., Winooski, Vt., USA). To detect macromolecule m-to-s movement across the mucosa, FD4 (0.1 mM) was applied into the mucosal bath and serosal FD4 content was assessed by FITC fluorescence intensity measurement.

TLR-4 reporter cell assay. Since the LAL test may overestimate LPS levels due to nonspecific detection of monophosphoryl lipid A (MPLA) (66), and FITC-LPS method measures LPS fluorescence without regard to the functional activity of LPS, experiments were carried out to determine whether the samples contained intact, functional LPS that is a preferential TLR-4 ligand. In order to confirm that intact LPS was measured in the samples used for measurements in the Ussing chamber study, a TLR4 reporter cell assay was used that consists of HEK293 cells co-transfected with murine TLR4, myeloid differentiation 2 and CD14 co-receptor genes and an inducible secreted embryonic alkaline phosphatase (SEAP) reporter gene with nuclear factor-kappa B (NF-kB) and activator protein 1 (AP-1) binding motifs (HEK-Blue mTLR-4, InvivoGen, San Diego, Calif.). Cells were harvested in a 96-well plate with standards and samples for 24 hr. The presence of LPS was detected as induced SEAP activity in the medium using HEK-Blue Detection (InvivoGen) by measuring absorbance at 655 nm with Synergy-2 24 hrs after the addition of standards and samples, according to the manufacturer's protocol. Ultrapure LPS from E. coli 055:B5 (LPS-B5 Ultrapure, InvivoGen) was used as standard at a range of 0.01-100 ng/ml.

Ussing chamber study: drug treatment. The following drugs were pretreated by addition to the mucosal (m) or serosal (s) bath 10 or 15 min before LPS addition to the mucosal bath: the lipid raft inhibitor MβCD (1 mM, m), the CD36 inhibitor SSO (0.1 mM, m), the muscarinic agonist CCh (10 μM, s), a competitive substrate for IAP (IAP inhibitor) GP (10 mM, m) (47), GLP-2 (100 nM, s), the dipeptidyl peptidase-4 (DPP4) inhibitor NVP-728 (10 μM, s) (36), the stable GLP-2 analog TDG (100 nM) (13), the insulin-like growth factor-1 (IGF-1) receptor tyrosine kinase inhibitor NVP-AEW-541 (10 μM, s) (22), the epidermal growth factor (EGF) tyrosine kinase inhibitor PD153035 (1 μM, s) (6), the nitric oxide (NO) synthase (NOS) inhibitor L-NAME (0.1 mM, s) and the selective VPAC1 antagonist PG97-269 (1 μM) (26).

In vivo small intestinal perfusion. Intraduodenal perfusions combined with PV and mesenteric lymph cannulation were carried out (1, 48). Under isoflurane anesthesia (2%), the animal was placed supine on a recirculating heating block system (Summit Medical Systems, Bend, Oreg., USA) in order to maintain body temperature at 36-37° C. Prewarmed saline was infused via the right femoral vein at 1.08 ml/h using a Harvard infusion pump (Harvard Apparatus, Holliston, Mass., USA). The abdomen was incised, and the PV was cannulated with a polyethylene (PE)-50 tube attached with 23G needle and fixed by methylacrylate adhesive at the insertion site. The tube was filled with heparinized saline enabling repeated blood sampling. PV samples (0.2 ml each) were collected every 15 min, followed by 0.2 ml flushes with heparinized saline. Next, the main trunk of the mesenteric lymph duct located between the celiac and superior mesenteric arteries was cannulated with another PE-50 tube attached with 23G needle and filled with saline and fixed by methylacrylate adhesive at the insertion site. The lymph solution was continuously collected into a 1.5-ml tube every 15 min. A polyethylene tube (diameter 5 mm) filled with PBS was inserted through the forestomach and tied at 0.5 cm caudal from the pyloric ring. After bolus perfusion of 2 ml PBS into the duodenum from the tube, followed by stabilization for ˜30 min, the time was set as t=0 min. FITC-LPS (50 μg/ml) in 2-ml PBS with or without OA (30 mM) and TCA (10 mM) was bolus perfused at t=0 min. In order to maintain lymph flow, 2-ml PBS was perfused every 30 min. PV and lymph samples were collected every 15 min for 90 min. Blood samples were collected in a 0.5-ml tube containing 1 μl each of EDTA (0.5 M), and immediately centrifuged at 5,000 × g for 5 min, after which the plasma was stored at −80° C. until use. The lymph solution was weighed to measure its volume and centrifuged, with the supernatant stored at −80° C. until use. At 90 min, after collection of PV and lymph samples, arterial blood was taken from abdominal aorta for a reference sample, followed by euthanasia by thoracotomy.

The appearance of FITC-LPS into the PV plasma and lymph was assessed by fluorescence measurement using Synergy-2. Fluorescence intensity in the t=0 sample as background was subtracted from the fluorescence values measured in other time point samples. For lymph samples, the FITC-LPS concentration was multiplied by the lymph output in order to calculate total FITC-LPS appearance. FITC-LPS appearance was expressed as PV FITC-LPS content (ng/ml) or FITC-LPS transport into lymph (ng/15 min). Furthermore, total FITC-LPS transport into the PV and lymph was assessed by calculating the area under the curve (AUC) during 0-90 min of FITC-LPS appearance (AUC₀₋₉₀) using the trapezoidal rule and expressed as AUC₀₋₉₀ of PV FITC-LPS (μg·min/ml) and AUC₀₋₉₀ of lymph FITC-LPS (μg).

Since repeated blood sampling is difficult in mice, one PV blood sample was taken. Under isoflurane anesthesia, prewarmed saline was infused sc at 0.1 ml/hr and the abdomen was incised. A polyethylene tube (diameter 2 mm) filled with PBS was inserted through the forestomach and secured with a silk suture 0.2 cm caudal from the pyloric ring. After bolus perfusion of 0.2 ml PBS into the duodenum from the tube, followed by stabilization for ˜30 min, the time was set as t=0. FITC-LPS (50 μg/ml) in PBS with or without OA (30 mM) and TCA (10 mM) was perfused 0.2-ml at t=0 min. At t=15 min, PV blood and abdominal arterial blood were collected, followed by euthanasia by thoracotomy. Autofluorescence of PV plasma from the pooled non-treated fasted control mice (average 613.5 at sensitivity 80 set in Synergy-2, n=6) were subtracted from the PV plasma samples to eliminate the background autofluorescence in plasma.

Intestinal perfusion study: drug treatment. The lipid raft inhibitor MβCD (1 mM), the CD36 inhibitor SSO (1 mM), and PL81 (3%) were added to the initial perfusion of 2-ml PBS at t=−30 min, followed by their addition to the luminal FITC-LPS/OA/TCA solution at t=0 min.

Effect of TDG on intestinal LPS transport. The stable, DPP4-resistant analog of GLP-2, TDG (13) was used to examine the acute effect of GLP-2 on LPS transport into PV and mesenteric lymph in vivo. TDG (50 μg/kg) was bolus injected iv 15 min before the luminal FITC-LPS/OA/TCA perfusion (at t=−15 min). L-NAME was added to the first perfusion of 2 ml PBS (at t=−30 min). PG97-269 (0.3 mg/kg) was bolus injected iv 20 min before the luminal FITC-LPS/OA/TCA perfusion (at t=−20 min).

In vivo confocal imaging of LPS transport in mouse jejunum. In vivo confocal multiphoton microscopic imaging was performed in order to clarify whether LPS is transported via the transcellular or the paracellular pathway using a modification (3, 31). Due to the limited size of the microscopic stage, mice were used for this study. Under isoflurane anesthesia (2%), the abdomen was incised and the mid-jejunum (˜2 cm) was exposed. After the lumen was filled with 1 ml prewarmed PBS, the anterior wall of the jejunum was incised using an electric cautery. After gently rinsing the lumen with prewarmed PBS, the animal was placed prone with the jejunal mucosa face down in to the incubation chamber (Warner Instrument, Hamden, Conn., USA) filled with prewarmed PBS, set on the microscopic stage of a Leica TCS-SP2 AOBS inverted confocal and multi-photon microscope (Leica, Mannheim, Germany), equipped with an argon laser and three helium-neon lasers and a Spectra-Physics MaiTai® picosecond pulsed infrared laser (Mountain View, Calif.) set at 780 nm for two photon and infrared excitation. After stabilization for ˜15 min, the mucosa was exposed to FITC-LPS (50 μg/ml) in PBS for 15 min, followed by FITC-LPS plus OA (30 mM)/TCA (10 mM) in PBS for 15 min. The mucosal images were taken by excitation at 488 nm or by two-photon excitation at 780 nm with emission at 500-550 nm. For a negative control, the cell-impermeant red fluorescence dye SNARF-5F (10 μM; Molecular Probes, Eugene, Oreg., USA) was also added in the mucosal solution, and the images were taken by two-photon excitation at 780 nm and emission at 640 nm.

Statistics. Values are expressed as mean±SEM. The number of animals in each experimental group was n=4-6. Statistical analysis was performed using GraphPad® Prism 6 (La Jolla, Calif., USA) using one-way ANOVA or two-way ANOVA followed by Tukey's multiple comparisons, or using Mann-Whitney test, if applicable. Differences were considered significant when P values were <0.05.

Results. Transmucosal jejunal LPS transport in vitro. Mucosa-to-serosa (m-to-s) LPS transport was tested with or without added luminal OA in a muscle-stripped mucosa-submucosa preparation of rat jejunum using the LAL test. The jejunal mucosa was luminally exposed to LPS alone (10 μg/ml) from t=0 min to t=15 min, followed by the addition of vehicle (PBS), TCA (0.1 mM) or OA+TCA from t=15 min to t=45 min. LPS alone or LPS+TCA only in the mucosal bath had no effect on LPS concentrations in the serosal bath, whereas further addition of OA (3, 10 or 30 mM) in the mucosal bath dose-dependently increased LPS concentrations in the serosal bath at t=30 min that were sustained at t=45 min (FIG. 6A), suggesting that luminal LCFA exposure increases m-to-s transport of LPS. Mucosal application of LPS (10 μg/ml) with OA (30 mM) plus TCA (0.1 mM) was thus used in the following experiments. OA+TCA without LPS in the lumen had no effect on serosal LPS concentrations (FIG. 6B). LPS content in the mucosal bath at t=0 min measured by LAL test was 14.0 ±2.1 EU/ml (n=6), equivalent to 2.5 ng/ml according to the conversion of 1 EU/ml=0.18 ng/ml LPS determined by the LAL test used. Since LPS was added at 10 μg/ml in the mucosal bath, these results suggest that endogenous LPS present in the lumen of the jejunal mucosa did not contribute to serosal LPS concentrations.

Since LCFA absorption is partially mediated by the LCFA translocator CD36 (65) and by lipid raft-related endocytosis (14), next, the effects of the CD36 inhibitor SSO, and the lipid raft inhibitor MβCD were examined on LPS transport during OA/TCA exposure. Pretreatment with SSO (0.1 mM) had no effect on LPS transport at t=15 min, whereas OA/TCA-augmented LPS transport (t=30-45 min) was abolished by the addition of luminal SSO (FIG. 6C), suggesting that luminal LPS transport is mediated at least in part by CD36. MβCD (1 mM) also inhibited OA/TCA-augmented LPS transport (FIG. 6D), suggesting that lipid rafts also mediate LPS transport. These results also suggest that LPS absorption occurs during OA/TCA exposure via the transcellular pathway rather than by the paracellular pathway. In contrast, pretreatment with serosal CCh (10 which empties goblet cells by stimulating mucus secretion, increased LPS transport at t=15 min, and further increased LPS transport during the OA/TCA exposure period (FIG. 6E), suggesting that emptied goblet cells may transport LPS, regardless the presence of OA/TCA in the lumen, consistent with LPS uptake through emptied goblet cells (32). Furthermore, the addition of a competitive substrate for IAP, GP (10 mM) in the mucosal bath further increased LPS transport at t=45 min (FIG. 6F), suggesting that IAP inhibition increases the bioavailability of LPS in the lumen, since IAP detoxifies LPS by de-phosphorylation of lipid A (71), bearing in mind that the LAL test as a functional assay detects the activity of LPS that has intact lipid A, but also detects MPLA as having ˜50% of the activity of intact lipid A (66). LPS transport in the Ussing chamber (LPS alone at t=15 min and LPS+OA+TCA at t=45 min) is summarized in FIG. 6G. Note that the value represents accumulated serosal [LPS], since the serosal bath volume was constant. SSO, MβCD and GP had no effect on LPS transport in the absence of OA/TCA, whereas SSO and MβCD inhibited, but GP enhanced LPS transport in the presence of OA/TCA. In contrast, CCh increased LPS movement independently of luminal OA/TCA.

Since the LAL test may be confounded components in the medium, OA/TCA-induced LPS transport was examined using FITC-LPS with its serosal appearance measured by FITC fluorescence intensity. FITC-LPS alone in the lumen had no effect on serosal LPS content for 45 min, whereas luminal application of OA/TCA increased serosal LPS content measured by FITC fluorescence intensity, the effect abolished by the addition of SSO or MβCD (FIG. 7A), mirroring the results measured by LAL test in FIGS. 6C and D. To further validate the detection of LPS m-to-s transport, the same samples of FIG. 7A were analyzed by the TLR4 reporter cell assay, that showed that the detectable range of LPS-B5 Ultrapure was 0.1-30 ng/ml. Using this method, LPS m-to-s transport was increased during OA/TCA exposure and abolished by SSO or MβCD, whereas no change was observed in the FITC-LPS alone group (FIG. 7B), compatible with the

FITC measurements (FIG. 7A). The detected LPS levels were ˜0.3-˜10 ng/ml and ALPS levels were up to ˜1.5 ng/ml, much less than the FITC measurements (FIG. 7A), and relatively less than the LAL measurements (FIG. 6), by which ALPS levels were up to ˜30 EU/ml, which is equivalent to ˜5.4 ng/ml. These results suggest that intact LPS is present in the serosal solution, although transported LPS is likely degraded. Nevertheless, the methods similarly detected ALPS m-to-s increase during OA/TCA exposure that was consistently inhibited by membrane transport inhibitors, confirming that LPS transport measured by the LAL test is comparable with the fluorescence measurements of FITC-LPS or TLR4 bioactivity assay in our in vitro experimental conditions.

Epithelial permeability during OA/TCA exposure in jejunal mucosa in vitro. To examine whether paracellular permeability changes were contributed to the enhanced LPS transport observed during OA/TCA exposure, transepithelial electrical resistance (TEER) was measured in parallel with FITC-LPS or FITC-dextran 4000 (FD4) m-to-s movement in the jejunal mucosa.

Luminal addition of OA/TCA increased serosal [FITC-LPS] (FIG. 8A), whereas TEER was unchanged throughout the experiments (FIG. 8B). Luminal application of FD4 and LPS with or without further addition of OA/TCA increased serosal [FD4] (FIG. 8C) without any change in TEER (FIG. 8D), suggesting that FD4 transport in the jejunum occurs tonically regardless of the luminal presence of OA/TCA. These results suggest that luminal OA/TCA exposure increases LPS transport without associated paracellular permeability changes for electrolytes and macromolecules.

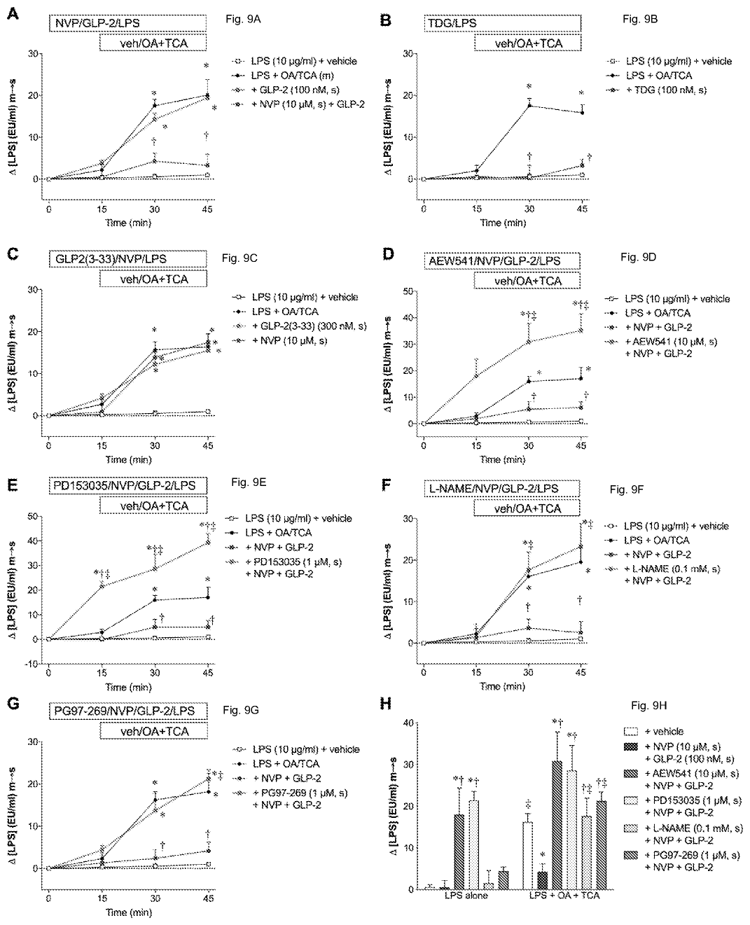

Effect of GLP-2 on jejunal LPS transport in vitro. Next, it was tested whether GLP-2 acutely affects LPS m-to-s transport in the small intestine. Serosal application of GLP-2 (100 nM) alone at t=−15 min had no effect on OA/TCA-augmented LPS m-to-s transport, whereas further addition of the dipeptidyl peptidase-4 (DPP4) inhibitor NVP-728 (10 μM) to GLP-2 inhibited LPS transport during OA/TCA exposure period (FIG. 9A), suggesting that prolonging the half-life of GLP-2 is important to acutely inhibit LPS transport during OA/TCA exposure in the Ussing chamber. Using the stable GLP-2 analog teduglutide (TDG, 100 nM), which inhibited LPS transport during OA/TCA exposure in the absence of DPP4 inhibition (FIG. 9B), confirmed that GLP-2 acutely affects LPS m-to-s transport in the small intestine. To examine whether endogenous GLP-2 is involved in OA/TCA-augmented LPS transport, the effects of a GLP-2 receptor partial agonist/antagonist GLP-2(3-33) (68) and NVP-728 alone on LPS transport was tested. Serosal application of GLP-2(3-33) (300 nM) or NVP-728 (10 μM) had no effect on LPS transport at t=15 min, or OA/TCA-augmented LPS transport at t=30-45 min (FIG. 7C), suggesting that endogenous GLP-2 is not involved in OA/TCA-augmented LPS transport. To explore mechanisms underlying the inhibitory effects of GLP-2 on LPS transport during OA/TCA exposure, the pathways downstream of GLP-2 receptor activation including IGF-1 and EGF, and neural pathways involving NO and VIP, were investigated. The IGF-1 receptor tyrosine kinase inhibitor NVP-AEW-541 (10 μM, s) abolished the effect of GLP-2 on LPS transport, and further increased the amount of LPS transport (FIG. 9D). The EGF receptor tyrosine kinase inhibitor PD153035 (1 μM, s) increased the amount of LPS transport prior to OA/TCA exposure, and further increased the amount of LPS transport after addition of OA/TCA (FIG. 9E). These results suggest that IGF-1 and EGF signaling in addition to their chronic pro-proliferative effects acutely regulate basal LPS permeability. In contrast, L-NAME (0.1 mM, s) (FIG. 9F) and the VPAC1 antagonist PG97-269 (1 μM, s) (FIG. 9G) had no effect on the amount of LPS transport at t=15 min, but reversed the inhibitory effect of GLP-2 on LPS transport during OA/TCA exposure. These results are summarized in FIG. 9H, demonstrating that NVP-AEW-541 and PD153035 increased the amount of LPS transport in the absence of luminal OA/TCA exposure, whereas L-NAME and PG97-269 reversed the inhibitory effect of GLP-2 on the amount of LPS transport during OA/TCA exposure, suggesting that neural NO and VIP-VPAC1 signals are downstream of the mechanism by which GLP-2 inhibits LPS transport.

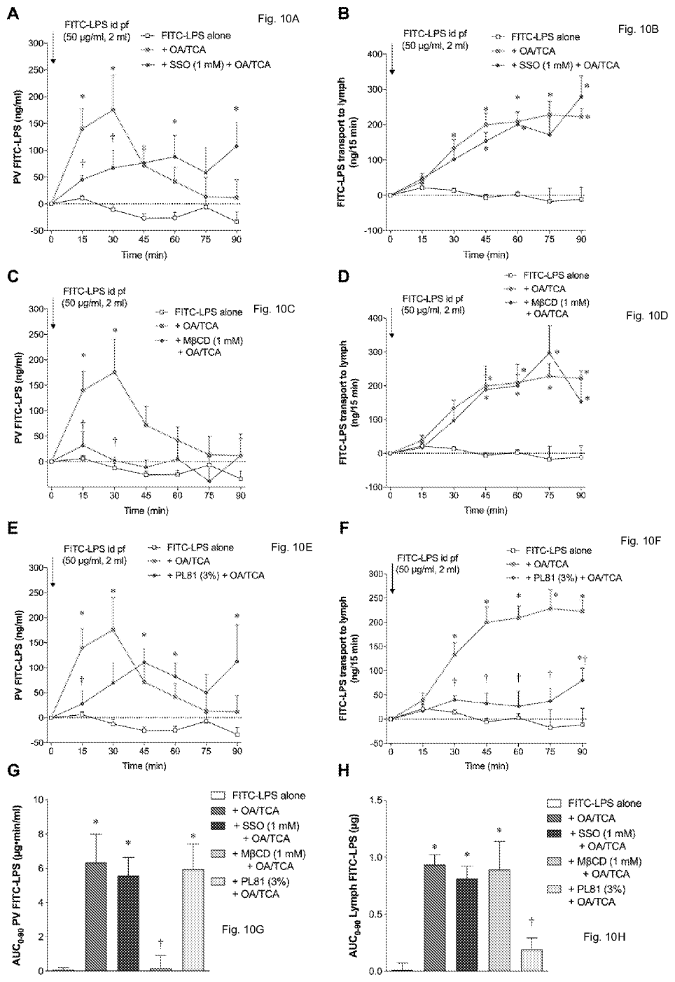

FITC-LPS transport during LCFA absorption in vivo. To further study the mechanism of enhanced LPS transport during lipid exposure, FITC-LPS transport was measured during LCFA absorption in vivo in anesthetized rats. The dynamics of FITC-LPS movement was examined from the lumen to the PV and lymph during OA/TCA exposure. Low-level FITC-LPS appearance in the PV plasma (FIG. 10A) and lymph (FIG. 10B) was present after an intraduodenal bolus perfusion of FITC-LPS alone (50 μg/ml in 2-ml PBS). In contrast, bolus perfusion of FITC-LPS with OA (30 mM)/TCA (10 mM) in 2 ml PBS rapidly increased FITC-LPS appearance in the PV at t=15 and 30 min, which then declined to baseline, whereas FITC-LPS gradually appeared in the lymph with a peak at t=60 min, reaching a plateau at t=60-90 min, the latter consistent with the dynamics of chylomicron transport during physiological LCFA absorption (70). These results show that LPS transport during OA/TCA exposure is biphasic; rapid transport into the PV, followed by a more delayed transport into the lymph.

Next, the effects of lipid transport inhibitors were examined on FITC-LPS transport into the PV and lymph. Pretreatment with the CD36 inhibitor SSO (1 mM), followed by intraduodenal perfusion of the OA/TCA solution inhibited rapid LPS transport to the PV at t =15 and 30 min (FIG. 10A), whereas SSO had no effect on FITC-LPS transport into the lymph (FIG. 10B). Pretreatment and co-perfusion of the lipid raft inhibitor MβCD (1 mM) abolished FITC-LPS transport into the PV (FIG. 10C), but had no effect on FITC-LPS transport into the lymph (FIG. 10D). Furthermore, pretreatment and co-perfusion of the chylomicron synthesis inhibitor PL81 (3%) reduced FITC-LPS transport into the PV (FIG. 10E), and inhibited FITC-LPS transport into the lymph (FIG. 10F). These results suggest that the CD36- and lipid raft-mediated lipid absorption pathways are involved in rapid LPS transport into the PV, followed by gradual FITC-LPS transport into the lymph by LPS incorporation into chylomicrons, consistent with a previous report (23). Total LPS transport into the PV and the lymph was also assessed by calculation of AUC₀₋₉₀. OA/TCA exposure increased AUC₀₋₉₀ of PV FITC-LPS (FIG. 10G) and AUC₀₋₉₀ of lymph FITC-LPS (FIG. 10H), compared with the FITC-LPS alone group. Increased AUC₀₋₉₀ of PV FITC-LPS was abolished by MβCD treatment, whereas SSO and PL81 failed to affect overall FITC-LPS transport into the PV (FIG. 10G), although SSO and PL81 partially blunted the early rise of FITC-LPS transport into the PV at t=15 min (FIGS. 10A, E). In contrast, OA/TCA-augmented AUC₀₋₉₀ of lymph FITC-LPS was reduced by PL81 treatment (FIG. 10H).

These results also showed that total FITC-LPS transport into the lymph was ˜1 μg, with ˜1% of applied LPS in the lumen (100 μg). Assuming PV blood flow is ˜10 ml/min for a ˜250 g body weight rat, since reported PV blood flow values are ˜19 ml/min for 526 g rat and 20 ml/min 367 g rat (62, 72), ˜60 μg (˜60%) of added LPS was transported into the PV during a 90 min FITC-LPS exposure, suggesting that most of the luminal FITC-LPS was transported into the PV rather than into the lymph.

The effect of TDG on FITC-LPS transport was also examined in vivo in order to confirm the Ussing chamber results. Pretreatment with TDG (50 μg/kg, iv) at t=−15 min abolished FITC-LPS transport into the PV (FIG. 11A), whereas FITC-LPS transport to the lymph was rapidly increased at t=15 and 30 min (FIG. 11B). TDG-enhanced LPS transport into the lymph was accompanied by a rapid increase of lymphatic output at t=0-30 min, compared with non-pretreated controls (FIG. 11C), suggesting that TDG acutely enhances flow dynamics including mucosal blood and lymphatic flow, and then increases the number of FITC-LPS-containing chylomicrons in the lymph. Pretreatment with and co-perfusion of L-NAME (0.1 mM) reduced the inhibitory effect of TDG on FITC-LPS transport into the PV (FIG. 11A) and inhibited the TDG-related augmentation of FITC-LPS transport into the lymph (FIG. 11B) with reduced lymph output (FIG. 11C). Pretreatment with PG97-269 (0.3 mg/kg, iv) at t=−20 min had no effect on TDG-related inhibition on FITC-LPS transport into the PV (FIG. 11D), but inhibited the TDG-related augmentation of FITC-LPS transport into the lymph (FIG. 11E) with reduced lymph output (FIG. 11F). These results suggest that TDG acutely inhibits OA/TCA-induced FITC-LPS transport into the PV via an NO-mediated pathway, and enhances OA/TCA-induced FITC-LPS transport into the lymph via NO and VIP-VPAC1 pathways by increasing overall lymphatic output. TDG treatment reduced OA/TCA-augmented AUC₀₋₉₀ of PV FITC-LPS (FIG. 11G). L-NAME treatment reversed the TDG-induced reduction of AUC₀₋₉₀ PV FITC-LPS, whereas PG97-269 had no effect. TDG had no significant effect on OA/TCA-augmented AUC₀₋₉₀ of lymph FITC-LPS (FIG. 11H), although TDG increased FITC-LPS transport into the lymph at 15-30 min (FIGS. 11B, E). L-NAME and PG97-269 treatment reduced AUC₀₋₉₀ of lymph FITC-LPS, compared with the +TDG group (FIG. 11H). These results show that TDG inhibits LPS entry into the PV and initially accelerates but has a little overall effect on LPS transport into the lymph.

Direct visualization of FITC-LPS uptake into epithelial cells in vivo in mouse jejunal mucosa. To directly distinguish intracellular uptake from paracellular diffusion of macromolecules in the small intestinal mucosa in vivo, the localization of FITC-LPS in mouse jejunal mucosa was visualized using in vivo two-photon confocal microscopy, since single-photon conventional confocal microscopy was not able to penetrate the tissue deeply enough to visualize the intracellular localization of fluorescence at the basolateral pole of the villous cells. For these experiments, mouse jejunal mucosa was exposed to luminal FITC-LPS solution (50 μg/ml) alone for 15 min, followed by to FITC-LPS with OA (30 mM) plus TCA (10 mM) under isoflurane anesthesia. Localization of FITC-LPS was imaged using single-photon or two-photon confocal microscope. The results demonstrate that a single-photon confocal microscopic image of FITC-LPS incubated with OA plus TCA showed no clear fluorescent signal in the villous cells, although the lumen had a strong FITC signal. In contrast, a two-photon confocal image of the same area showed intracellular FITC signals in the villous cells (vertical optical sections of villous cells; arrowheads) as well as FITC signals in the cytosol and at the basolateral pole of the villous cells contrasted with negatively-stained nuclei. Luminal incubation with FITC-LPS (50 μg/ml) alone in PBS over the jejunal mucosa for 15 min showed no apparent intracellular localization of fluorescent signals with faint staining at the surface of villous cells. In contrast, the addition of OA (30 mM) plus TCA (10 mM) to luminal FITC-LPS solution for 5 min rapidly stained the intracellular space of the villous cells with negative staining of the outlines of each villous cell. Deeper scanning also revealed that FITC signals were present at the basolateral pole of the villous cells with negative staining of the nuclei. Black spots were also observed in the villi with no stained structure, corresponding to the previously reported cell shedding-induced epithelial gaps (74). Furthermore, FITC-LPS was not visualized in the intercellular spaces, in contrast to in vivo confocal laser endomicroscopy of sodium fluorescein in human intestine (39).

These results show that luminal FITC-LPS is rapidly absorbed via the transcellular pathway by jejunal villous cells in the presence of OA/TCA rather than absorbed via the paracellular route. Co-incubation with a cell-impermeant red fluorescent dye SNARF-5F revealed negative intracellular staining, further confirming the transcellular uptake of FITC-LPS.

FITC-LPS transport into the PV in mice. To confirm whether acute LPS transport into the PV during LCFA exposure occurs in mice, FITC-LPS (50 μg/ml) solution (0.2 ml) with or without OA (30 mM)/TCA (10 mM) was bolus perfused from the duodenum in mice, thereafter PV blood was collected at t=15 min. FITC-LPS levels in the PV plasma were higher in FITC-LPS+OA/TCA groups compared with the FITC-LPS alone group, confirming that luminal FITC-LPS in the presence of OA/TCA is acutely transported into the PV in mice, similar to the results obtained in rats.

Discussion. The dynamics of LPS transport in rodent small intestine was studied during lipid absorption in vitro and in vivo, observing that LPS is acutely transported from the mucosal to the serosal side via CD36- and lipid raft-mediated mechanisms during LCFA exposure, and that LPS transport in vivo is biphasic with rapid transport into the PV followed by slower transport into the lymph; the former related to lipid rafts and partially via CD36 and chylomicron formation, whereas the latter is mediated by the chylomicron-dependent LCFA uptake pathway. AUC analysis demonstrated that recovery of luminally-added FITC-LPS was ˜60% into the PV and ˜1% into the lymph, suggesting that most of luminal LPS is rapidly transported into the PV during lipid absorption. Furthermore, the results show that exogenous GLP-2 combined with DPP4 inhibition or the stable GLP-2 analog TDG inhibited the acute phase of LPS transport during LCFA exposure in vitro and rapid LPS transport into the PV in vivo, whereas TDG accelerated LPS transport into the lymph by increasing the rate of lymphatic output early in the time course. This is the first study directly demonstrating physiological absorption of LPS from the small intestinal lumen in vivo during LCFA absorption via the chylomicron-mediated absorption pathway into the lymph. This study also describes for the first time a LPS transport pathway into the PV. Furthermore, the results demonstrated that GLP-2 modifies LPS transport during LCFA absorption acutely rather than as a consequence of the long-term chronic pro-proliferative effects of GLP-2. These results provide important insight into the mechanism by which the intestinal mucosa handles a lipophilic, highly potent and toxic bacterial substance present in high luminal concentrations such as LPS in order to avoid activating undesired systemic inflammatory pathways, how the augmentation of LPS transport by dietary fat contributes to endotoxemia, and how the endogenous peptide GLP-2 modifies LPS absorption via the PV and chylomicron pathways.

Two methods were used to assess LPS movement from the lumen; LPS activity measured by the LAL test in vitro and FITC-LPS uptake in vivo. There are a variety of methods used to measure LPS in biological samples that have advantages and disadvantages; the LAL test, LPS antibody-based ELISA kit, bioassay using TLR4-expressing cells with a reporter gene, and labeled LPS molecules such as FITC-LPS. Since there is no one test that has overall superiority, measurements were chosen based on the requirements of each system used. In the Ussing chamber, the serosal solution of muscle-stripped intestinal mucosa contained a substantial amount of autofluorescence, whereas blood plasma generally contains interfering factors for the LAL test or anti-coagulant components that may affect the enzymatic assay on which the LAL test is based. The LAL test is probably the simplest and best way to detect intact lipid A activity bearing in mind that lipid A activity may be masked by the presence of chylomicrons, especially in the lymph, since chylomicrons inhibit lipid A activity (15). Therefore, the LAL test was used for in vitro studies and FITC-LPS was used for in vivo studies. Despite these dissimilar approaches, the results demonstrated consistent transcellular LPS transport via lipid absorption-related pathways. Furthermore, the comparability among the LAL test, FITC-LPS flux, and the TLR4 reporter cell assay in the measurement of LPS transport during OA/TCA exposure in the jejunum in vitro was confirmed.

In health, LPS is limited to the intestinal lumen due to the presence of the multiple defense mechanisms including luminal antimicrobial peptides, mucins, and brush border IAP in addition to intercellular tight junctions that are unlikely to allow passage of a 15-20 kDa lipophilic monomer such as LPS. These observations notwithstanding, paracellular permeability to much smaller solutes such as FITC-dextran 4000 has been used a surrogate for gut barrier function in endotoxin-related diseases, suggesting that the paracellular pathway may become dominant in the presence of inflammation, drugs, or other pathological states (35, 52, 61). Nevertheless, a high-fat diet increases systemic LPS concentrations in mice and humans (8, 18), chylomicrons contain LPS (23), and LPS absorption is enhanced by chylomicron formation (23), together suggesting that LPS is assimilated as part of the physiologic absorption pathway of dietary LCFA. Chronic exposure to even low levels of circulating LPS due to chronic dietary intake of excessive fat, combined with increased amounts of luminal Gram-negative bacteria may be sufficient to activate inflammatory cascades in adipocytes, pancreatic islet β cells, and hepatocytes, changes that have been related to truncal obesity, hypertension, cardiovascular disease, type 2 diabetes, hyperlipidemia, and fatty liver as components of the metabolic syndrome (17, 49).

One can assume that LPS as a lipophilic large molecule may cross the epithelium when the paracellular spaces are widened due to dysfunction or disruption of the tight junctional complex or following epithelial cell injury. Yet, paracellular permeability markers such as TEER and FD4 movement do not always behave in parallel. The results showed that FITC-LPS crossed the jejunal mucosa during OA/TCA exposure without changes in FD4 movement and TEER, suggesting that LPS is transported via the transcellular pathway rather than by the paracellular route, although FD4 crossed the jejunal mucosa somewhat non-specifically. Furthermore, one can question how the colonic mucosa regulates LPS movement, since the colonic lumen contains >99% of the bacterial LPS in the gut lumen. Since the focus was on small intestinal LPS transport in the present study, further study is needed to examine colonic LPS movement and to clarify how sepsis is suppressed despite massive colonic luminal LPS concentrations even in the presence of mucosal injury, even though modest endotoxemia has been reported in inflammatory bowel disease (53). It thus appears that a small background of LPS entry into the PV and lymph occurs via established lipid uptake pathways in the small intestine subject to neurohormonal regulation whereas the colon, due to its steep m-to-s LPS gradient, serves as an effective barrier to LPS entry under physiological and pathological conditions.

On the basis of in vitro data, several small intestinal transport mechanisms are implicated in LPS transport (29): endocytosis mediated by lipid raft-dependent or independent pathways, goblet cell-associated antigen passage (GAP), chylomicron-dependent pathways, and paracellular pathways. The in vitro results demonstrated that LPS was transported in the presence of luminal LCFA via CD36- and lipid raft-mediated mechanisms, suggesting that transport pathways used for the uptake of LCFAs also transport luminal LPS into epithelial cells. The transcellular uptake of luminal LPS was also confirmed directly by in vivo two-photon confocal microscopic imaging, wherein FITC-LPS in the presence of luminal LCFA was rapidly localized to the villous cell cytoplasm, but not to the paracellular space. Furthermore, in vivo perfusion studies documented rapid FITC-LPS appearance in the PV via CD36- and lipid raft-mediated mechanisms, followed by a gradual appearance in the lymph dependent on chylomicron synthesis. CD36 and the LPS receptors CD14 and TLR4 are present in lipid rafts (14) (54). Lipid raft disruption by MβCD decreases the LPS permeation coefficient without any change in paracellular permeability, suggesting the involvement of caveola-dependent mechanisms (43), consistent with prior in vitro studies.

The results also showed that there were two major routes of LPS entry from the small intestine during lipid absorption: transport into the PV, and transport into the lymph with chylomicrons. Furthermore, PV LPS transport was rapid and transient compared to the gradual and sustained increase of lymphatic LPS transport. Although rapid PV LPS transport was partially inhibited by CD36 inhibition and abolished by lipid raft disruption, these interventions did not affect delayed lymphatic LPS transport, suggesting that LPS entry into the villous cells is via the physiological lipid absorption pathways that take up LCFAs (and presumably MCFAs) from the intestinal lumen, whereas the exit pathway from the villous cells and transfer into the blood capillaries are dissimilar to chylomicron transport into the lymph. Due to their particle size, chylomicrons enter to the central lacteals and villous lymphatic capillaries that have wide slit-like openings in the lymphatic endothelium (69). Free LPS or smaller lipid particles may exit from the villous cells and enter the blood capillaries to the PV. Another possibility is that epithelial or subepithelial lipoprotein lipase may dissociate initially transported chylomicrons with subsequent release of LPS from chylomicrons, similar to the mechanism by which lipoprotein lipase releases LCFAs from TG in chylomicrons, which then activate LCFA receptors on enteroendocrine L cells (56).

Interestingly, absorbed, circulating LPS is taken up by hepatocytes for clearance and excretion into the bile (45). Therefore, although lipid absorption is potentially harmful due to concomitant absorption of LPS, earlier studies have reported that chylomicrons inhibit lipid A activity (15) and enhance LPS uptake by hepatocytes and LPS excretion into the bile (58). Thus, LPS uptake by the chylomicron pathway has intrinsic protective mechanisms that detoxify LPS and facilitate LPS removal from the circulation. Bile salts also inhibits lipid A activity (60), suggesting that bile in the intestinal lumen and chylomicrons in the circulation prevent functional LPS lipid A from activating the immune systems in the mucosa, the circulation, and the liver. Therefore, accelerated chylomicron transport incorporating LPS, which eventually enters the systemic circulation via the thoracic duct, may induce less toxicity than does ‘free’ LPS transport into the PV, which may directly activate TLR4 expressed on Kupffer cells and hepatic stellate cells, promoting hepatic inflammation as part of the ‘gut-liver axis’ (63). These results suggest that chronic reduction of PV LPS entry may prevent low-grade inflammation in the liver, thus reducing the development of fat accumulation and inflammation (steatohepatitis), which has been linked to the activation of inflammatory cascades via TLR4 activation (9). Increased PV LPS levels present in experimental colitis augment the development of steatohepatitis (21) whereas reduction of intestinal LPS by oral antibiotics improves hepatic fibrosis with reducing intestinal permeability (11).

GLP-2 is released from enteroendocrine L cells in response to meals, likely through signaling via bacterial metabolites such as SCFAs, whose receptors are expressed on L cells (1). Although the stable GLP-2 analog TDG is approved for the treatment of short-gut syndrome due to its chronic trophic effects on the gut epithelium, it also has acute effects that are less well studied, including accelerating intestinal lipid uptake at 60-90 min (33) and enterocyte chylomicron release at 60 min (10), and reducing paracellular permeability of the mouse jejunum starting ˜4 hr after injection (5). Here, the results described herein demonstrated that exogenous GLP-2 combined with DPP4 inhibition or TDG acutely inhibited LCFA-facilitated LPS transport in vitro in Ussing chambered intestine. Luminal LCFA and bile acids may increase endogenous GLP-2 release via activation of LCFA receptors free fatty acid receptor (FFA) 1 and 4, and bile acid receptor TGR5 on L cells (1, 36, 59). Basolateral LPS or systemically administered LPS increases GLP-1 release in vitro or in vivo (37, 42). Therefore, endogenous GLP-2 released by OA/TCA or transported LPS may be involved in the in vitro study. Nevertheless, a GLP-2 receptor antagonist or DPP4 inhibitor alone had no effect on OA/TCA-augmented LPS transport, suggesting that the effect of endogenous GLP-2 is minimal. The inhibitory effects of GLP-2 were mediated at the least by NO and VIP-VPAC1 pathways, details of which remain to be determined. Neuronal VIP may regulate macromolecular permeability by increased expression of the intercellular junction protein zonula occludens-1 (51), although the results suggest that LPS is transported via the transcellular pathway rather than by the paracellular pathway, at least during OA/TCA exposure. The results described herein also confirmed the acute inhibitory effects of TDG on LPS transport into the PV via the NO pathway. The results showed that GLP-2-induced NO production suppressed LCFA-associated LPS transport in vitro, suggesting that NO directly regulates enterocyte LPS uptake from the lumen or release from the basolateral membranes. Since NO is involved in GLP-2-mediated chylomicron release from enterocytes (34), these results suggest that TDG may redirect LPS transport into the PV to LPS transport into the lymphatics via the NO pathway. Still, the vast majority of luminally added LPS was transported into the PV, and TDG-induced increase of FITC-LPS transport into the lymph was transient and did not affect overall LPS transport, suggesting that TDG-NO pathway directly modifies epithelial LPS entry or intracellular/subepithelial LPS movement. The direct effects of NO on LPS movement from the lumen to the subepithelial space remain to be determined.

TDG acutely augmented LPS transport into the lymph probably by increasing lymph output via NO and VIP-VPAC1-mediated pathways, since GLP-2 increases superior mesenteric arterial blood flow possibly via NO and VIP release (28); increased intestinal microcirculatory flow enhances lymph output (46), due to increased capillary pressure that subsequently increases interstitial hydrostatic pressure (27). Although the reduction of PV LPS entry with increased chylomicron-mediated LPS absorption into the lymph was previously unknown, chylomicron-bound LPS in the lymphatics is less toxic than ‘free’ LPS in the PV due to the many detoxifying defenses inherent in the chylomicron pathway. Further study will clarify the effect of TDG on the development of high fat diet-induced fatty liver.

LPS may also be transported through emptied goblet cells in mouse ileum (32), termed the goblet cell-associated antigen passage (GAP) (44). The data described herein showed that CCh pretreatment, which empties goblet cells, increased LPS transport regardless of prior lipid exposure, suggesting that LPS may pass through emptied goblet cells possibly via a GAP-related luminal lipid-independent mechanism. Another possibility is that, since CCh increases mucus secretion and anion secretion, CCh-induced changes of the pre-epithelial mucus barrier to macromolecule diffusion and unstirred layer conditions may contribute to the increase of LPS transport. The results also showed that the IAP inhibitor GP increased the transport of active LPS during lipid exposure, consistent with IAP-mediated LPS dephosphorylation of lipid A with consequent detoxification (71). Orally-administered IAP inhibits metabolic changes induced by high-fat diets (38), ameliorates chemically-induced colitis in a murine model (57) and attenuates alcohol-induced fatty liver in mice (30), consistent with LPS detoxification. Since IAP activity is increased at alkaline pH (4), and since the rate of HCO₃⁻ secretion is maximal in the duodenum (2), the jejunum as the proximate downstream intestinal segment and primary locus of LCFA absorption has the high luminal pH and expression of brush border IAP activity necessary to maximally detoxify LPS concurrent with LCFA absorption, serving as another protective mechanism to reduce systemic LPS toxicity. Since GLP-2 stimulates duodenal HCO₃⁻ secretion via the NO and VIP pathways (73), it is possible that GLP-2 reduces bioactive LPS entry by reducing LPS transport into the PV, as well as by increasing HCO₃⁻ secretion that in turn increases the rate of lipid A dephosphorylation by IAP while augmenting chylomicron transport. Further study will clarify this possibility by monitoring bioactive lipid A transport in the PV and in chylomicrons.

In conclusion, the findings disclosed herein have demonstrated the dynamics of LPS transport during lipid exposure in the small intestine in vitro and in vivo demonstrating at least two distinct uptake pathways and finding no evidence for paracellular LPS transport in the absence of an induced paracellular permeability increase. Since LPS is involved in the pathogenesis of the metabolic syndrome, sepsis, and more, CD36, lipid rafts, and GLP-2 receptors may be the therapeutic targets for the prevention of LPS-related disease.

### REFERENCES

1. Akiba Y, Inoue T, Kaji I, Higashiyama M, Narimatsu K, Iwamoto K, Watanabe M, Guth P H, Engel E, Kuwahara A, and Kaunitz J D. Short-chain fatty acid sensing in rat duodenum. J Physiol 593: 585-599, 2015.

2. Akiba Y, and Kaunitz J D. Duodenal luminal chemosensing; Acid, ATP, and nutrients. Curr Pharm Des 20: 2760-2765, 2014.

3. Akiba Y, and Kaunitz J D. Regulation of intracellular pH and blood flow in rat duodenal epithelium in vivo. Am J Physiol Gastrointest Liver Physiol 276: G293-G302, 1999.

4. Akiba Y, Mizumori M, Guth P H, Engel E, and Kaunitz J D. Duodenal brush border intestinal alkaline phosphatase activity affects bicarbonate secretion in rats. Am J Physiol Gastrointest Liver Physiol 293: G1223-G1233, 2007.

5. Benjamin M A, McKay D M, Yang P C, Cameron H, and Perdue MH. Glucagon-like peptide-2 enhances intestinal epithelial barrier function of both transcellular and paracellular pathways in the mouse. Gut 47: 112-119, 2000.

6. Bos M, Mendelsohn J, Kim Y M, Albanell J, Fry D W, and Baselga J. PD153035, a tyrosine kinase inhibitor, prevents epidermal growth factor receptor activation and inhibits growth of cancer cells in a receptor number-dependent manner. Clin Cancer Res 3: 2099-2106, 1997.

7. Cani P D, Amar J, Iglesias M A, Poggi M, Knauf C, Bastelica D, Neyrinck A M, Fava F, Tuohy K M, Chabo C, Waget A, Delmee E, Cousin B, Sulpice T, Chamontin B, Ferrieres J, Tanti J F, Gibson G R, Casteilla L, Delzenne N M, Alessi M C, and Burcelin R. Metabolic endotoxemia initiates obesity and insulin resistance. Diabetes 56: 1761-1772, 2007.

8. Cani P D, Possemiers S, Van de W T, Guiot Y, Everard A, Rottier O, Geurts L, Naslain D, Neyrinck A, Lambert D M, Muccioli G G, and Delzenne N M. Changes in gut microbiota control inflammation in obese mice through a mechanism involving GLP-2-driven improvement of gut permeability. Gut 58: 1091-1103, 2009.

9. Csak T, Velayudham A, Hritz I, Petrasek J, Levin I, Lippai D, Catalano D, Mandrekar P, Dolganiuc A, Kurt-Jones E, and Szabo G. Deficiency in myeloid differentiation factor-2 and toll-like receptor 4 expression attenuates nonalcoholic steatohepatitis and fibrosis in mice. Am J Physiol Gastrointest Liver Physiol 300: G433-G441, 2011.

10. Dash S, Xiao C, Morgantini C, Connelly P W, Patterson B W, and Lewis G F. Glucagon-like peptide-2 regulates release of chylomicrons from the intestine. Gastroenterology 147: 1275-1284, 2014.

11. Douhara A, Moriya K, Yoshiji H, Noguchi R, Namisaki T, Kitade M, Kaji K, Aihara Y, Nishimura N, Takeda K, Okura Y, Kawaratani H, and Fukui H. Reduction of endotoxin attenuates liver fibrosis through suppression of hepatic stellate cell activation and remission of intestinal permeability in a rat non-alcoholic steatohepatitis model. Mol Med Rep 11: 1693-1700, 2015.

12. Drucker D J, Erlich P, Asa S L, and Brubaker P L. Induction of intestinal epithelial proliferation by glucagon-like peptide 2. Proc Natl Acad Sci USA 93: 7911-7916, 1996.

13. Drucker D J, Yusta B, Boushey R P, DeForest L, and Brubaker P L. Human [Gly 2]GLP-2 reduces the severity of colonic injury in a murine model of experimental colitis. Am J Physiol 276: G79-G91, 1999.

14. Ehehalt R, Sparla R, Kulaksiz H, Herrmann T, Fullekrug J, and Stremmel W. Uptake of long chain fatty acids is regulated by dynamic interaction of FAT/CD36 with cholesterol/sphingolipid enriched microdomains (lipid rafts). BMC Cell Biol 9: 45, 2008.

15. Eichbaum E B, Harris H W, Kane J P, and Rapp J H. Chylomicrons can inhibit endotoxin activity in vitro. J Surg Res 51: 413-416, 1991.

16. Erridge C. The capacity of foodstuffs to induce innate immune activation of human monocytes in vitro is dependent on food content of stimulants of Toll-like receptors 2 and 4. Br J Nutr 105: 15-23, 2011.

17. Erridge C. Diet, commensals and the intestine as sources of pathogen-associated molecular patterns in atherosclerosis, type 2 diabetes and non-alcoholic fatty liver disease. Atherosclerosis 216: 1-6, 2011.

18. Erridge C, Attina T, Spickett C M, and Webb D J. A high-fat meal induces low-grade endotoxemia: evidence of a novel mechanism of postprandial inflammation. Am J Clin Nutr 86: 1286-1292, 2007.

19. Freudenberg M A, Freudenberg N, and Galanos C. Time course of cellular distribution of endotoxin in liver, lungs and kidneys of rats. Br J Exp Pathol 63: 56-65, 1982.

20. Fullerton J N, Segre E, De Maeyer R P, Maini A A, and Gilroy D W. Intravenous endotoxin challenge in healthy humans: an experimental platform to investigate and modulate systemic inflammation. J Vis Exp 2016.

21. Gäbele E, Dostert K, Hofmann C, Wiest R, Scholmerich J, Hellerbrand C, and Obermeier F. DSS induced colitis increases portal LPS levels and enhances hepatic inflammation and fibrogenesis in experimental NASH. J Hepatol 55: 1391-1399, 2011.

22. García-Echeverriá C, Pearson M A, Marti A, Meyer T, Mestan J, Zimmermann J, Gao J, Brueggen J, Capraro H G, Cozens R, Evans D B, Fabbro D, Furet P, Porta D G, Liebetanz J, Martiny-Baron G, Ruetz S, and Hofmann F. In vivo antitumor activity of NVP-AEW541-A novel, potent, and selective inhibitor of the IGF-IR kinase. Cancer Cell 5: 231-239, 2004.

23. Ghoshal S, Witta J, Zhong J, de V W, and Eckhardt E. Chylomicrons promote intestinal absorption of lipopolysaccharides. J Lipid Res 50: 90-97, 2009.

24. Goldberg R F, Austen W G, Jr., Zhang X, Munene G, Mostafa G, Biswas S, McCormack M, Eberlin K R, Nguyen J T, Tatlidede H S, Warren H S, Narisawa S, Millan J L, and Hodin R A. Intestinal alkaline phosphatase is a gut mucosal defense factor maintained by enteral nutrition. Proc Natl Acad Sci USA 105: 3551-3556, 2008.

25. Goudriaan J R, Dahlmans V E, Febbraio M, Teusink B, Romijn J A, Havekes L M, and Voshol P J. Intestinal lipid absorption is not affected in CD36 deficient mice. Mol Cell Biochem 239: 199-202, 2002.

26. Gourlet P, De N P, Cnudde J, Waelbroeck M, and Robberecht P. In vitro properties of a high affinity selective antagonist of the VIP1 receptor. Peptides 18: 1555-1560, 1997.

27. Granger D N, Perry M A, Kvietys P R, and Taylor A E. Capillary and interstitial forces during fluid absorption in the cat small intestine. Gastroenterology 86: 267-273, 1984.

28. Guan X, Karpen H E, Stephens J, Bukowski J T, Niu S, Zhang G, Stoll B, Finegold M J, Holst J J, Hadsell D, Nichols B L, and Burrin D G. GLP-2 receptor localizes to enteric neurons and endocrine cells expressing vasoactive peptides and mediates increased blood flow. Gastroenterology 130: 150-164, 2006.

29. Guerville M, and Boudry G. Gastrointestinal and hepatic mechanisms limiting entry and dissemination of lipopolysaccharide into the systemic circulation. Am J Physiol Gastrointest Liver Physiol 311: G1-G15, 2016.

30. Hamarneh S R, Kim B M, Kaliannan K, Morrison S A, Tantillo T J, Tao Q, Mohamed M M R, Ramirez J M, Karas A, Liu W, Hu D, Teshager A, Gul S S, Economopoulos K P, Bhan A K, Malo M S, Choi M Y, and Hodin R A. Intestinal Alkaline Phosphatase Attenuates Alcohol-Induced Hepatosteatosis in Mice. Dis Dig Sci 62: 2021-2034, 2017.

31. Hirokawa M, Takeuchi T, Chu S, Akiba Y, Wu V, Guth P H, Engel E, Montrose M H, and Kaunitz J D. Cystic fibrosis gene mutation reduces epithelial cell acidification and injury in acid-perfused mouse duodenum. Gastroenterology 127: 1162-1173, 2004.

32. Howe S E, Lickteig D J, Plunkett K N, Ryerse J S, and Konjufca V. The uptake of soluble and particulate antigens by epithelial cells in the mouse small intestine. Plos One 9: e86656, 2014.

33. Hsieh J, Longuet C, Maida A, Bahrami J, Xu E, Baker C L, Brubaker P L, Drucker D J, and Adeli K. Glucagon-like peptide-2 increases intestinal lipid absorption and chylomicron production via CD36. Gastroenterology 137: 997-1005, 1005, 2009.

34. Hsieh J, Trajcevski K E, Farr S L, Baker C L, Lake E J, Taher J, Iqbal J, Hussain M M, and Adeli K. Glucagon-like peptide 2 (GLP-2) stimulates postprandial chylomicron production and postabsorptive release of intestinal triglyceride storage pools via induction of nitric oxide signaling in male hamsters and mice. Endocrinology 156: 3538-3547, 2015.

35. Hurni M A, Noach A B, Blom-Roosemalen M C, de Boer A G, Nagelkerke J F, and Breimer D D. Permeability enhancement in Caco-2 cell monolayers by sodium salicylate and sodium taurodihydrofusidate: assessment of effect-reversibility and imaging of transepithelial transport routes by confocal laser scanning microscopy. J Pharmacol Exp Ther 267: 942-950, 1993.

36. Inoue T, Wang J H, Higashiyama M, Rudenkyy S, Higuchi K, Guth P H, Engel E, Kaunitz J D, and Akiba Y. Dipeptidyl peptidase IV inhibition potentiates amino acid- and bile acid-induced bicarbonate secretion in rat duodenum. Am J Physiol Gastrointest Liver Physiol 303: G810-G816, 2012.

37. Kahles F, Meyer C, Mollmann J, Diebold S, Findeisen H M, Lebherz C, Trautwein C, Koch A, Tacke F, Marx N, and Lehrke M. GLP-1 secretion is increased by inflammatory stimuli in an IL-6-dependent manner, leading to hyperinsulinemia and blood glucose lowering. Diabetes 63: 3221-3229, 2014.

38. Kaliannan K, Hamarneh S R, Economopoulos K P, Nasrin A S, Moaven O, Patel P, Malo N S, Ray M, Abtahi S M, Muhammad N, Raychowdhury A, Teshager A, Mohamed M M, Moss A K, Ahmed R, Hakimian S, Narisawa S, Millan J L, Hohmann E, Warren H S, Bhan A K, Malo M S, and Hodin R A. Intestinal alkaline phosphatase prevents metabolic syndrome in mice. Proc Natl Acad Sci USA 110: 7003-7008, 2013.

39. Kiesslich R, Duckworth C A, Moussata D, Gloeckner A, Lim L G, Goetz M, Pritchard D M, Galle P R, Neurath M F, and Watson A J. Local barrier dysfunction identified by confocal laser endomicroscopy predicts relapse in inflammatory bowel disease. Gut 61: 1146-1153, 2012.

40. Koyama I, Matsunaga T, Harada T, Hokari S, and Komoda T. Alkaline phosphatases reduce toxicity of lipopolysaccharides in vivo and in vitro through dephosphorylation. Clin Biochem 35: 455-461, 2002.

41. Lappin D F, Sherrabeh S, and Erridge C. Stimulants of Toll-like receptors 2 and 4 are elevated in saliva of periodontitis patients compared with healthy subjects. J Clin Periodontol 38: 318-325, 2011.

42. Lebrun L J, Lenaerts K, Kiers D, Pais de Barros J P, Le Guern N, Plesnik J, Thomas C, Bourgeois T, Dejong C H C, Kox M, Hundscheid I H R, Khan N A, Mandard S, Deckert V, Pickkers P, Drucker D J, Lagrost L, and Grober J. Enteroendocrine L cells sense LPS after gut barrier injury to enhance GLP-1 secretion. Cell reports 21: 1160-1168, 2017.

43. Mani V, Hollis J H, and Gabler N K. Dietary oil composition differentially modulates intestinal endotoxin transport and postprandial endotoxemia. Nutr Metab (Loud) 10: 6, 2013.

44. McDole J R, Wheeler L W, McDonald K G, Wang B, Konjufca V, Knoop K A, Newberry R D, and Miller M J. Goblet cells deliver luminal antigen to CD103+ dendritic cells in the small intestine. Nature 483: 345-349, 2012.

45. Mimura Y, Sakisaka S, Harada M, Sata M, and Tanikawa K. Role of hepatocytes in direct clearance of lipopolysaccharide in rats. Gastroenterology 109: 1969-1976, 1995.

46. Miura S, Tashiro H, Kurose I, Suematsu M, Serizawa H, Sekizuka E, Nagata H, Yoshioka M, and Tsuchiya M. Intestinal microcirculatory changes during fat absorption and the effect of cholecystokinin inhibitor. Am J Physiol 262: G399-G404, 1992.

47. Mizumori M, Ham M, Guth P H, Engel E, Kaunitz J D, and Akiba Y. Intestinal alkaline phosphatase regulates protective surface microclimate pH in rat duodenum. J Physiol 587: 3651-3663, 2009.

48. Mizumori M, Meyerowitz J, Takeuchi T, Lim S, Lee P, Supuran C T, Guth P H, Engel E, Kaunitz J D, and Akiba Y. Epithelial carbonic anhydrases facilitate PCO₂ and pH regulation in rat duodenal mucosa. J Physiol 573: 827-842, 2006.

49. Moreira A P, Texeira T F, Ferreira A B, Peluzio M C, and Alfenas R C. Influence of a high-fat diet on gut microbiota, intestinal permeability and metabolic endotoxaemia. Br J Nutr 108: 801-809, 2012.

50. Nassir F, Wilson B, Han X, Gross R W, and Abumrad N A. CD36 is important for fatty acid and cholesterol uptake by the proximal but not distal intestine. J Biol Chem 282: 19493-19501, 2007.

51. Neunlist M, Toumi F, Oreschkova T, Denis M, Leborgne J, Laboisse C L, Galmiche J P, and Jarry A. Human ENS regulates the intestinal epithelial barrier permeability and a tight junction-associated protein ZO-1 via VIPergic pathways. Am J Physiol Gastrointest Liver Physiol 285: G1028-G1036, 2003.

52. Parlesak A, Schafer C, Schutz T, Bode J C, and Bode C. Increased intestinal permeability to macromolecules and endotoxemia in patients with chronic alcohol abuse in different stages of alcohol-induced liver disease. J Hepatol 32: 742-747, 2000.

53. Pastor R O, Lopez San R A, Albeniz A E, de la Hera M A, Ripoll S E, and Albillos M A. Serum lipopolysaccharide-binding protein in endotoxemic patients with inflammatory bowel disease. Inflamm Bowel Dis 13: 269-277, 2007.

54. Plociennikowska A, Hromada-Judycka A, Borzecka K, and Kwiatkowska K. Co-operation of TLR4 and raft proteins in LPS-induced pro-inflammatory signaling. Cell Mol Life Sci 72: 557-581, 2015.

55. Poltorak A, He X, Smirnova I, Liu M Y, Van H C, Du X, Birdwell D, Alejos E, Silva M, Galanos C, Freudenberg M, Ricciardi-Castagnoli P, Layton B, and Beutler B. Defective LPS signaling in C3H/HeJ and C57BL/10ScCr mice: mutations in Tlr4 gene. Science 282: 2085-2088, 1998.

56. Psichas A, Larraufie P F, Goldspink D A, Gribble F M, and Reimann F. Chylomicrons stimulate incretin secretion in mouse and human cells. Diabetologia 60: 2475-2485, 2017.

57. Ramasamy S, Nguyen D D, Eston M A, Alam S N, Moss A K, Ebrahimi F, Biswas B, Mostafa G, Chen K T, Kaliannan K, Yammine H, Narisawa S, Millan J L, Warren H S, Hohmann E L, Mizoguchi E, Reinecker H C, Bhan A K, Snapper S B, Malo M S, and Hodin R A. Intestinal alkaline phosphatase has beneficial effects in mouse models of chronic colitis. Inflamm Bowel Dis 17: 532-542, 2011.

58. Read T E, Harris H W, Grunfeld C, Feingold K R, Calhoun M C, Kane J P, and Rapp J H. Chylomicrons enhance endotoxin excretion in bile. Infect Immun 61: 3496-3502, 1993.

59. Reimann F, Tolhurst G, and Gribble F M. G-protein-coupled receptors in intestinal chemosensation. Cell Metab 15: 421-431, 2012.

60. Rudbach J A, Anacker R L, Haskins W T, Johnson A G, Milner K C, and Ribi E. Physical aspects of reversible inactivation of endotoxin. Ann N Y Acad Sci 133: 629-643, 1966.

61. Salzman A L, Wang H, Wollert P S, Vandermeer T J, Compton C C, Denenberg A G, and Fink M P. Endotoxin-induced ileal mucosal hyperpermeability in pigs: role of tissue acidosis. Am J Physiol 266: G633-G646, 1994.

62. Schaffner D, von Elverfeldt D, Deibert P, Lazaro A, Merfort I, Lutz L, Neubauer J, Baumstark M W, Kreisel W, and Reichardt W. Phase-contrast MR flow imaging: A tool to determine hepatic hemodynamics in rats with a healthy, fibrotic, or cirrhotic liver. Journal of magnetic resonance imaging: JMRI 46: 1526-1534, 2017.

63. Seki E, De M S, Osterreicher C H, Kluwe J, Osawa Y, Brenner D A, and Schwabe R F. TLR4 enhances TGF-beta signaling and hepatic fibrosis. Nat Med 13: 1324-1332, 2007.

64. Shim J, Moulson C L, Newberry E P, Lin M R, Xie Y, Kennedy S M, Miner J H, and Davidson N O. Fatty acid transport protein 4 is dispensable for intestinal lipid absorption in mice. J Lipid Res 50: 491-500, 2009.

65. Stahl A, Hirsch D J, Gimeno R E, Punreddy S, Ge P, Watson N, Patel S, Kotler M, Raimondi A, Tartaglia L A, and Lodish H F. Identification of the major intestinal fatty acid transport protein. Mol Cell 4: 299-308, 1999.

66. Takayama K, Qureshi N, Raetz C R, Ribi E, Peterson J, Cantrell J L, Pearson F C, Wiggins J, and Johnson A G. Influence of fine structure of lipid A on Limulus amebocyte lysate clotting and toxic activities. Infect Immun 45: 350-355, 1984.

67. Taveira da Silva A M, Kaulbach H C, Chuidian F S, Lambert D R, Suffredini A F, and Danner R L. Brief report: shock and multiple-organ dysfunction after self-administration of Salmonella endotoxin. N Engl J Med 328: 1457-1460, 1993.

68. Thulesen J, Knudsen L B, Hartmann B, Hastrup S, Kissow H, Jeppesen P B, Orskov C, Hoist J J, and Poulsen S S. The truncated metabolite GLP-2 (3-33) interacts with the GLP-2 receptor as a partial agonist. Regul Pept 103: 9-15, 2002.

69. Tso P, and Balint J A. Formation and transport of chylomicrons by enterocytes to the lymphatics. Am J Physiol 250: G715-G726, 1986.

70. Tso P, Buch K L, Balint J A, and Rodgers J B. Maximal lymphatic triglyceride transport rate from the rat small intestine. Am J Physiol 242: G408-G415, 1982.

71. Vaishnava S, and Hooper L V. Alkaline phosphatase: keeping the peace at the gut epithelial surface. Cell Host Microbe 2: 365-367, 2007.

72. van der Hoven B, van Pelt H, Swart E L, Bonthuis F, Tilanus H W, Bakker J, and Gommers D. Noninvasive functional liver blood flow measurement: comparison between bolus dose and steady-state clearance of sorbitol in a small-rodent model. Am J Physiol Gastrointest Liver Physiol 298: G177-181, 2010.

73. Wang J H, Inoue T, Higashiyama M, Guth P H, Engel E, Kaunitz J D, and Akiba Y. Umami receptor activation increases duodenal bicarbonate secretion via glucagon-like peptide-2 release in rats. J Pharmacol Exp Ther 339: 464-473, 2011.

74. Watson A J, Chu S, Sieck L, Gerasimenko O, Bullen T, Campbell F, McKenna M, Rose T, and Montrose M H. Epithelial barrier function in vivo is sustained despite gaps in epithelial layers. Gastroenterology 129: 902-912, 2005.

75. Zhang R, Miller R G, Gascon R, Champion S, Katz J, Lancero M, Narvaez A, Honrada R, Ruvalcaba D, and McGrath M S. Circulating endotoxin and systemic immune activation in sporadic amyotrophic lateral sclerosis (sALS). J Neuroimmunol 206: 121-124, 2009.

### Example 3: GLP-2 Acutely Ameliorates Endotoxin-Related Intestinal Paracellular Permeability in Rats

Abstract. Background: Circulating endotoxin (lipopolysaccharide; LPS) increases the gut paracellular permeability. It was assessed whether glucagon-like peptide-2 (GLP-2) acutely reduces LPS-related increased intestinal paracellular permeability by a mechanism unrelated to its intestinotrophic effect.

Methods: Small intestinal paracellular permeability was assessed in vivo by measuring the appearance of intraduodenally perfused FITC-dextran 4000 (FD4) into the portal vein (PV) in rats 1-24 hr after LPS treatment (5 mg/kg, ip). The effect of a stable GLP-2 analog teduglutide (TDG) was also examined on FD4 permeability.

Results: FD4 movement into the PV was increased 6 hr, not 1 or 3 hr after LPS treatment, with increased PV GLP-2 levels and increased mRNA expressions of proinflammatory cytokines and proglucagon in the ileal mucosa. Co-treatment with a GLP-2 receptor antagonist enhanced PV FD4 concentrations. PV FD4 concentrations at 24 hr were enhanced compared to FD4 concentrations at 6 hr, reduced by exogenous GLP-2 treatment given 6 hr after LPS treatment. TDG reduced FD4 permeability 3 or 6 hr after LPS treatment in the 6 hr study. The effect of TDG was reversed by the VPAC1 antagonist PG97-269 or L-NAME, not by EGF or IGF1 receptor inhibitors. TDG also reduced FD4 permeability 6 and 12 hr, not 0 or 24 hr after LPS treatment.

Conclusions: Systemic LPS releases endogenous GLP-2, reducing LPS-related increased permeability. The therapeutic window of exogenous GLP-2 administration is at minimum within 6-12 hr after LPS treatment. Exogenous GLP-2 treatment is of value in the prevention of increased paracellular permeability associated with endotoxemia.

Introduction. Lipopolysaccharide (LPS; endotoxin) is a lipophilic pathogenic factor derived from gut Gram-negative bacteria. Translocation of LPS or Gram-negative bacteria across the gut causes endotoxemia, associated with sepsis and sever acute illness, which triggers systemic inflammation. Systemic inflammation also increases intestinal paracellular permeability, associated with endotoxemia, further aggravating systemic inflammation. Severe systemic inflammation associated with acute pancreatitis, fulminant hepatitis, burns, trauma, and severe infections is often complicated by endotoxemia [1]. Increased entry of LPS from the intestinal lumen enhances multiple organ injury. Chemically-induced dextran sulfate sodium (DSS) colitis increases portal vein LPS levels, increasing liver injury [2]. Reduction of intestinal luminal LPS by antibiotics reduces ischemia-induced lung injury [3]. Therefore, therapeutics that decrease ‘secondary’ endotoxemia that are aimed at reducing intestinal LPS transport may prevent the morbid complications of critical illness [4].

Glucagon-like peptide-2 (GLP-2) is an intestinotrophic hormone released from enteroendocrine L cells [5]. Chronic treatment with GLP-2 (twice a day for 14 days) prevents LPS translocation into the circulation [6], attributed to its pro-proliferative effects with increased expression of tight junction proteins. The intestinotrophic effects of GLP-2 are mediated by the release of growth factors, insulin-like growth factor-1 (IGF1) or epidermal growth factor (EGF) [7,8], mostly from the peri-epithelial mesenchymal syncytium that express GLP-2 receptors (GLP2R) [9,10]. In addition, GLP2R are also expressed on the enteric neurons of myenteric and submucosal plexuses that express nitric oxide (NO) synthase (NOS) and vasoactive intestinal peptide (VIP) [11], suggesting that GLP-2 acutely affects mucosal responses via NO and VIP release. NO and VIP increase mucosal blood flow, stimulate epithelial anion secretion, and reduce gut paracellular permeability [12-17]. Therefore, it was assessed whether GLP-2 acutely affects small intestinal paracellular permeability under systemic inflammation via NO and VIP pathways, rather than via the growth factor pathway.

Systemic LPS treatment releases GLP-1 from L cells directly or indirectly. In mice, a single dose of LPS time-dependently increased serum GLP-1 levels at ˜2hr accompanied by hyperinsulinemia and hypoglycemia in an interleukin (IL)-6-dependent manner [18]. Another group also reported that a single ip LPS injection increased plasma GLP-1 levels at 6 hr in mice [19]. Plasma GLP-1 levels are higher in critically ill patients in intensive care unit (ICU) compared with healthy control subjects, and higher in sepsis patients in an ICU cohort than in non-septic patients [18]. LPS directly stimulates GLP-1 release from L cells via Toll-like receptor-4 (TLR4) activation at 3 hr in mice and at 1-24 hr in the L cell model cell lines, GLUTag cells and secretin tumor cell (STC)-1 cells [20]. Since equimolar amounts of GLP-1 and GLP-2 are released from stimulated L cells [21], LPS treatment may increase endogenous release of GLP-2 from L cells as well. Therefore, it was also assessed whether endogenous GLP-2 alters small intestinal paracellular permeability under systemic inflammation.

As described herein, the effects of endogenous and exogenous GLP-2 was examined on increased small intestinal paracellular permeability induced by systemic LPS treatment as a simple and reproducible model of acute inflammation [22,23]. Fluorescein isothiocyanate (FITC)-conjugated dextran 4kDa (FD4) was used as a model paracellular permeability marker [24] measuring FD4 appearance in the portal vein (PV) in rats in vivo in order to assess the dynamics of FD4 movement across the small intestinal mucosa after LPS treatment. The effects of a stable GLP-2 analog teduglutide (TDG) was further examined on FD4 permeability during LPS-induced systemic inflammation.

Materials and methods. Animal. Male Sprague-Dawley rats weighing 200-250 g (Harlan, San Diego, Calif., USA) were fed a pellet diet and water ad libitum. Some rats were fasted overnight with free access to water before the experiments, but some were fed ad libitum. Animals were euthanized by terminal exsanguination under deep isoflurane anesthesia, followed by thoracotomy.

Chemicals. Teduglutide (TDG, Shire Pharmaceuticals USA, Lexington, Mass., USA) was provided by the Pharmacy Service of the West Los Angeles Veterans Affairs Medical Center. Rat GLP-2, NVP-728, NVP-AEW-541 (AEW541) and PD153035 were obtained from Tocris Bioscience (Ellisville, Mo., USA). Rat GLP-2(3-33) was synthesized by Bachem Americas, Inc. (Torrance, Calif.). The VIP/pituitary adenylate cyclase-activating peptide (PACAP) receptor 1 (VPAC1) antagonist PG97-269; [Ac-His¹, D-Phe², Lys¹⁵, Arg¹⁶, Leu²⁷]-VIP(1-7)-GRF(8-27) (SEQ ID NO: 41) [25] was synthesized using solid-phase methodology according to the Fmoc-strategy using an automated peptide synthesizer (Model Pioneer, Thermo Fisher Scientific, Waltham, Mass., USA). The crude peptide was purified using reverse-phase high performance liquid chromatography (HPLC: Delta 600 HPLC System, Waters, Mass., USA) on a column of Develosil ODS-HG-5 (2×25 cm, Nomura Chemical Co., Ltd, Seto, Japan). The purity of each peptide was confirmed by analytical HPLC and matrix assisted laser desorption/ionization time of flight and mass spectrometry (MALDI-TOF MS) analysis. FD4, LPS (from E. coli 055:B5), N^{ω}-nitro-L-arginine methyl ester (L-NAME), and other chemicals were purchased from Sigma Chemical (St. Louis, Mo., USA). NVP-AEW-541 and PD153035 were dissolved in dimethyl sulfoxide (DMSO) for stock solution. The other chemicals were dissolved in distilled water in order to make a stock solution.

LPS treatment. Animals were treated with LPS (5 mg/kg, ip) once at 9 am. For the acute experiments, the animals were fasted overnight and were treated with LPS 1, 3, or 6 hr before the anesthesia for small intestinal perfusion of FD4 as described herein. For the 24-hr experiments, the animals fed ad libitum were treated with LPS 24 hr before anesthesia induction used for the perfusion study. As a control, saline was injected ip at the corresponding time before the experiments. The animal groups were expressed as control, LPS 1 hr, LPS 3 hr, LPS 6 hr, and LPS 24 hr.

Small intestinal perfusion. The small intestinal perfusion from the duodenum, and portal vein (PV) cannulation were prepared by the modified methods [26,27]. Under isoflurane anesthesia (2%), the animal was placed spine on a recirculating heating block system (Summit Medical Systems, Bend, Oreg., USA) in order to maintain body temperature at 36-37° C. Prewarmed saline was infused via the right femoral vein at 1.08 ml/h using a Harvard infusion pump (Harvard Apparatus, Holliston, Mass., USA). The abdomen was incised, and the PV was cannulated with a polyethylene (PE)-50 tube attached with 23G needle and fixed by methylacrylate adhesive at the insertion site. The tube was filled with heparinized saline enabling repeated blood sampling. Samples of 0.2 ml PV blood were collected every 15 min, followed by flushing with 0.2 ml heparinized saline. A polyethylene tube (diameter 5 mm) filled with PBS was inserted through the forestomach and tied at 0.5 cm caudal from the pyloric ring. After bolus perfusion of 2-ml phosphate buffer saline (PBS, 10 mM, pH 7.4) into the duodenum from the tube, followed by stabilization for ˜30 min, the time was set as t=0. FD4 (0.1 mM) in 10-ml PBS was slowly perfused at t=0 min for 30 sec. Blood samples were collected into a 0.5-ml tubes containing 1-μl each of EDTA (0.5 M) and the dipeptidyl peptidase-4 (DPP4) inhibitor NVP-728 (1 mM), and immediately centrifuged at 5,000× g for 5 min, after which the plasma was stored on ice or at −80 ° C. until use. At 90 min, after collection of PV samples, arterial blood was taken from abdominal aorta, followed by euthanasia by thoracotomy.

Appearance of FD4 into the PV and arterial plasma was assessed by fluorescence intensity measurement using a multi-mode microplate reader (Synergy-2, BioTek Instruments, Inc., Winooski, VT, USA). Fluorescence intensity in t=0 sample as background was subtracted from the fluorescence values measured in other time point samples. FD4 content was calculated according to the standard curve generated each time of measurement.

Drug treatment for the intestinal perfusion study. The following drugs were administered at the time after LPS treatment: the GLP2R partial agonist/antagonist GLP-2(3-33) (1 mg/kg; 280 nmol/kg, ip) [21,28] was given immediately after LPS treatment (0 hr after LPS treatment); GLP-2 (380 μg/kg; 100 nmol/kg, ip) was given 6 hr after LPS treatment; a stable GLP-2 analog TDG (50 μg/kg; 13.3 nmol/kg) [29] was ip injected 0, 3, 6, or 12 hr after LPS treatment or iv injected at t=0 min just before the perfusion of FD4 solution (6 or 24 hr after LPS treatment). In acute experiments, TDG was iv injected at t=0 min (6 hr after LPS treatment) with or without the pretreatment of the selective IGF1 receptor (IGF1R) tyrosine kinase inhibitor AEW541 (0.1 mg/kg, iv) [30], the selective EGF receptor (EGFR) tyrosine kinase inhibitor PD153035 (10 μg/kg, iv) [31] or PG97-269 (1 mg/kg, iv) at t=−10 min, or the co-perfusion of L-NAME (0.1 mM, pf) with FD4 solution.

GLP-2 measurement in PV plasma. GLP-2 content in PV plasma at t=0 min of control (overnight fasted), control (fed ad libitum), LPS 6 hr or LPS 24 hr group was measured using a GLP-2 ELISA kit (Phoenix Pharmaceuticals, Burlingame, Calif.) according to the manufacturer's protocol.

Real-time PCR. The mid-ileum (10 to 15 cm proximal from ileocecal junction) was removed from the animals of overnight fasted control and LPS 6 hr groups, and kept in a RNA stabilization solution (RNAlater, Quiagen, Valencia, Calif., USA) at 4° C. until use. The ileal mucosa was separated from muscle layers using sharp dissection under a stereomicroscope. RT-PCR was performed [27] with primers for rat proglucagon (Gcg), GLP2R, cyclooxygenase-2 (COX2), tumor necrosis factor α (TNFα), interleukin 6 (IL-6), EGF, IGF1, IGF1R and IGF2R, and for β-actin as internal control. The expression level was presented as fold induction per 10³ copies of β-actin by ΔCt method.

Immunofluorescence staining. Small pieces of intestine were immersed in Zamboni's fixative containing 2% paraformaldehyde and 0.2% picric acid in 0.1 M phosphate buffer (pH 7.4) overnight for 4° C. The fixed tissues were then submerged in 20% sucrose in PBS (pH 7.4) overnight at 4° C. and embedded in optimum cutting temperature compound. Frozen sections of 8-μm thickness were cut and placed on aminosilane-coated glass slides (Matsunami Glass USA Inc., Bellingham, Wash., USA). Sections were pretreated with 5% normal donkey serum in PBS, followed by incubation with primary antibodies; goat anti-GLP2R (Santa Cruz Biotechnology Inc., Santa Cruz, Calif., USA), rabbit anti-VIP (RayBiotech, Inc., Peachtree Corners, Ga., USA), or mouse anti-neuronal NOS (nNOS, Santa Cruz) overnight at 4° C. After rinsing in PBS, fluorescence-conjugated secondary antibodies (Molecular Probes, Eugene, Oreg.) were reacted for 2 hr at room temperature. The sections were counterstained with 4′,6-diamidino-2-phenylindole (DAPI) and covered with the mounting medium (Invitrogen, Carlsbad, Calif.). Immunofluorescence was imaged and captured using a confocal laser microscope (LSM710; Carl Zeiss GmbH, Jena, Germany).

Statistics. Values are expressed as mean±SEM. The number of animals in each experimental group was n=6. Statistical analysis was performed using ^{GraphPad}® Prism 6 (La Jolla, Calif., USA) using one-way ANOVA or two-way ANOVA followed by Dunnett's test or

Tukey's multiple comparisons. Unpaired Student's t-test was also used for two groups comparison. Differences were considered significant when P values were <0.05.

Results. Small intestinal FD4 permeability after LPS treatment. First, time-dependent changes were measured in small intestinal FD4 permeability from the lumen to the PV after LPS treatment (5 mg/kg, ip), reflecting intestinal paracellular permeability. There was no change in FD4 appearance into the PV in the control group. Compared with the control group, LPS treatment had no effect on PV FD4 levels 1 or 3 hr after LPS treatment, whereas PV FD4 levels were increased 6 hr after LPS treatment (FIG. 11A). Arterial FD4 levels at t=90 min (the end of experiments), also mirrored PV FD4 levels (FIG. 11B). Therefore, the LPS 6 hr model (6 hr after LPS treatment) was used to study the effects of acute drug treatment in the following experiments.

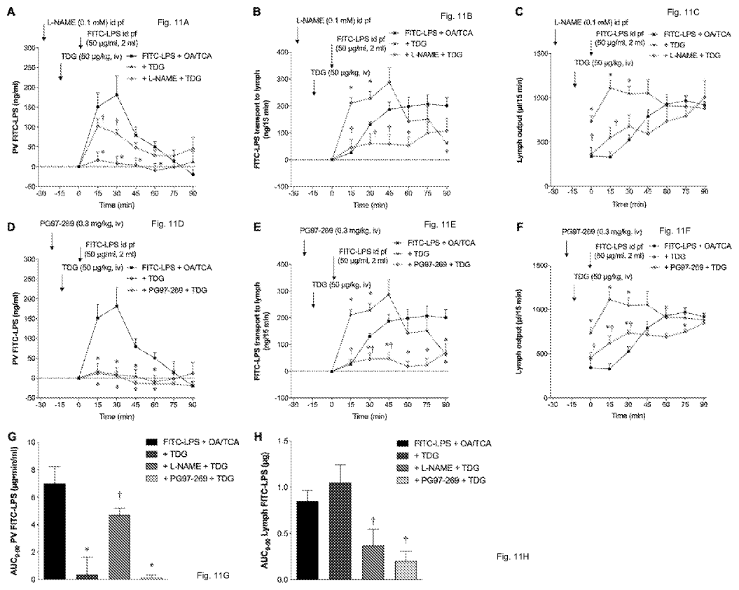

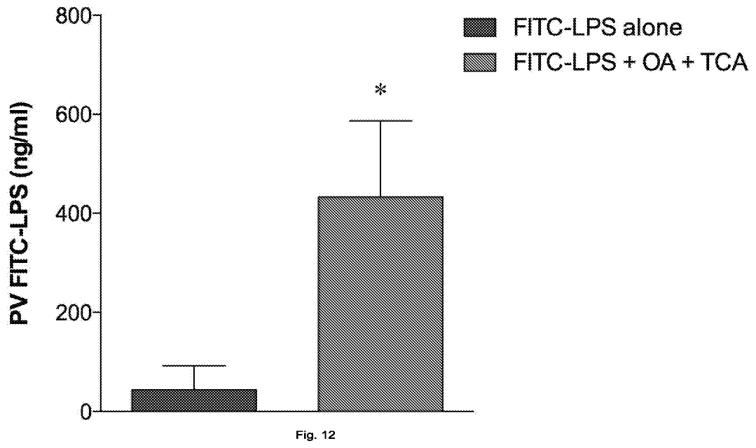

To test whether endogenous GLP-2 is involved in LPS-induced increased intestinal paracellular permeability, the animals were treated with the GLP2R antagonist, GLP-2(3-33) (1 mg/kg, ip) immediately after LPS ip injection. PV FD4 levels were increased at t=30 min, sustained to t=90 min in the LPS 6 hr group (FIG. 12A), accompanied by increased arterial FD4 levels at t=90 min at the end of the experiments (FIG. 12B), the latter possibly reflecting the accumulated FD4 transported from the small intestinal lumen. GLP-2(3-33) treatment further increased PV FD4 levels at t=60-90 min (FIG. 12A) and arterial FD4 levels at t=90 min (FIG. 12B), suggesting that endogenous GLP-2 released in response to LPS treatment reduces intestinal paracellular permeability and FD4 transport.

Next, FD4 permeability was examined 24 hr after LPS treatment (LPS 24 hr group). PV FD4 levels were increased 24 hr after LPS treatment (FIG. 13A) with increased arterial FD4 levels (FIG. 13B), higher than that in the LPS 6 hr group (LPS 6 hr 24.5 ±2.5 vs. LPS 24 hr 54.5±16.0, p<0.05 by unpaired Student's t-test). Exogenous GLP-2 treatment given 6 hr after LPS treatment (380 μg/kg, ip) reduced PV and arterial FD4 levels (FIG. 13A, B), suggesting that exogenous GLP-2 is therapeutically useful to reduce LPS-induced intestinal paracellular permeability, even when given 6 hr after LPS treatment.

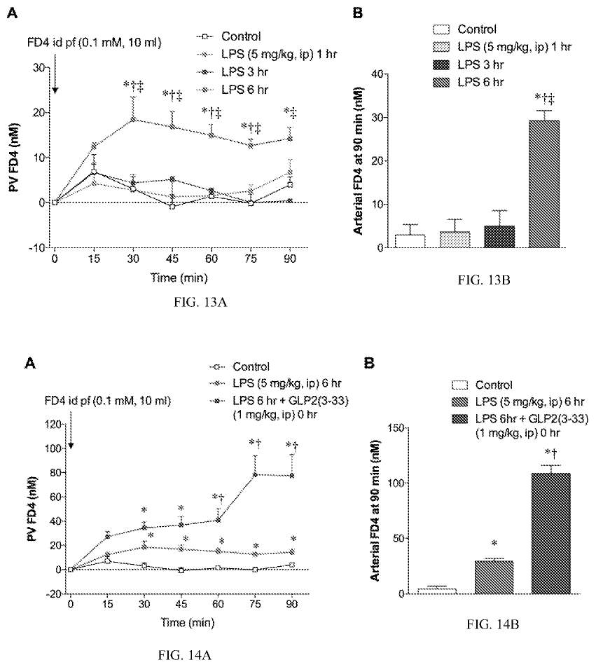

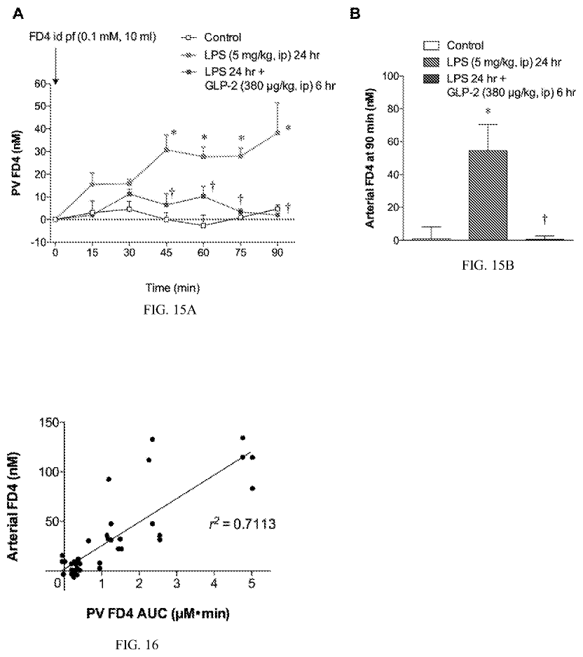

To clarify whether arterial FD4 levels reflect accumulated FD4 absorbed from the intestinal lumen to the PV, area under the curve (AUC) of PV FD4 levels during the 90 min period (μM⋅min) from FIGS. 13-15 were plotted against arterial FD4 levels at t=90 min (FIG. 16). This analysis demonstrated that PV FD4 AUC and arterial FD4 levels were well correlated (r²=0.7113), suggesting that arterial FD4 levels reflect the total transported FD4 amount into the PV.

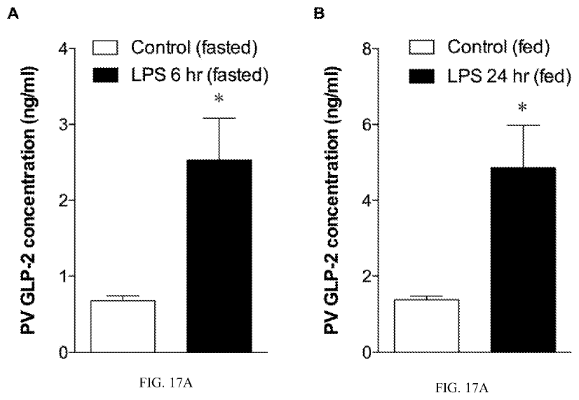

To confirm the involvement of endogenous GLP-2 during LPS-induced increased intestinal paracellular permeability, PV GLP-2 levels of the animals 6 and 24 hr after LPS treatment was measured. Compared with the corresponding control conditions (overnight fasted or fed ad libitum), LPS treatment increased PV GLP-2 levels 6 hr and 24 hr after LPS treatment (FIGS. 17A, B), suggesting that LPS directly or indirectly stimulates GLP-2 release from L cells of the intestine.

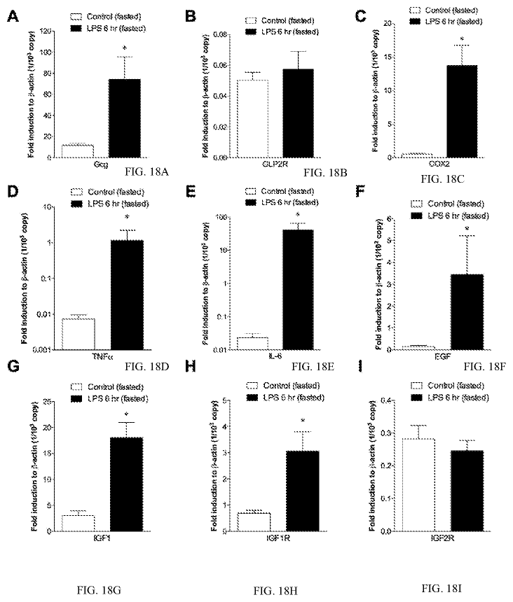

mRNA expression levels of proglucagon (Gcg) and proinflammatory mediators was also measured in the ileal mucosa by real-time PCR. Compared with the control group, expression of Gcg was increased in the ileal mucosa of the group treated with LPS at 6 hr (FIG. 18A), suggesting that LPS directly or indirectly upregulates Gcg expression in the ileal L cells, presumably to restore and further release GLP-2 and other proglucagon products. Expressions of COX-2 and the proinflammatory cytokines TNFα and IL-6 in the ileum were also upregulated by LPS treatment (FIGS. 18C-E). Furthermore, the expression of the growth factors EGF, IGF1, and IGFR1 that are believed to be downstream of GLP-2 receptor activation [7,8] (FIGS. 18F-H), but not GLP2R (FIG. 18B) or IGFR2 (FIG. 18I), were also upregulated, suggesting that GLP2R activation during LPS-induced inflammation upregulates its downstream signals.

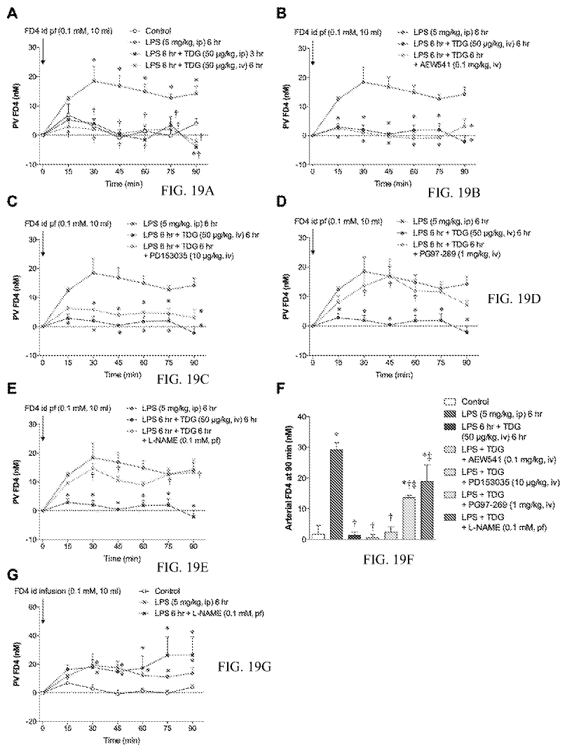

Effects of teduglutide (TDG) treatment on LPS-induced intestinal FD4 permeability. Next, the effects of TDG treatment on FD4 permeability in LPS 6 hr model was examined. TDG (50 μg/kg) was given 3 hr after LPS treatment (ip) or 6 hr after LPS treatment (iv at t=0 min), after which PV FD4 levels were measured 6 hr after LPS treatment. Compared with the non-treated LPS 6 hr group, TDG treatment at 3 hr (ip) or 6 hr (iv) after LPS treatment reduced PV FD4 levels (FIG. 19A), suggesting that TDG acutely improves LPS-induced increased intestinal FD4 permeability.

Furthermore, using the LPS 6 hr model treated with TDG 6 hr after LPS treatment, the downstream mediators involved in the inhibitory effect of TDG on FD4 permeability was assessed. Pretreatment with the selective IGF1R tyrosine kinase inhibitor AEW541 (0.1 mg/kg, iv) (FIG. 19B) or EGFR tyrosine kinase inhibitor PD153035 (10 μg/kg, iv) (FIG. 19C) had no effect on TDG-induced inhibitory effect on PV FD4 levels, whereas the selective VPAC1 antagonist PG97-269 (1 mg/kg, iv) (FIG. 19D) and an NOS inhibitor L-NAME (0.1 mM, pf) (FIG. 19E) reversed the TDG effect on PV FD4 levels. Arterial FD4 levels were reduced by PG97-269 and L-NAME, but were not affected by AEW541 or PD153035 (FIG. 19F), in agreement with the TDG-induced inhibitory effect on LPS-augmented PV FD4 levels. These results suggest that acute TDG effects on LPS-induced FD4 permeability are mediated by VIP and NO, but not by the growth factors IGF1 or EGF. Since NO derived from inducible NOS (iNOS) contributes to LPS-induced intestinal permeability increase [32,33], the effect of L-NAME was also examined alone on LPS-induced FD4 permeability. Luminal co-perfusion of L-NAME with FD4 had no effect on LPS-induced increases in PV FD4 levels at 6 hr (FIG. 19G), suggesting that luminal application of L-NAME may not affect iNOS activity in the small intestinal tissues or acute inhibition of iNOS may not reverse LPS-induced FD4 permeability.

The effects of TDG on FD4 permeability in an LPS 24 hr model was also tested. The increased PV FD4 levels 24 hr after LPS treatment (LPS 24 hr group) were reduced by TDG treatment 6 and 12 hr after LPS treatment (LPS 24 hr +TDG 6 hr group and +TDG 12 hr group), whereas TDG treatment immediately after LPS treatment (LPS 24 hr +TDG 0 hr group) or 24 hr after LPS treatment (LPS 24 hr +TDG 24 hr group) had little effect on the increased PV FD4 levels (FIG. 20A), suggesting that TDG given immediately or 24 hrs after LPS treatment are ineffective, whereas TDG given at 6-12 hr after LPS treatment is effective in reducing inflammation-increased paracellular permeability. Increased arterial FD4 levels in the LPS 24 hr group were inhibited by TDG treatment 6 hr and 12 hr after LPS treatment (LPS 24 hr +TDG 6 hr, and +TDG 12 hr), whereas TDG treatment 0 or 24 hr after LPS treatment (LPS 24 hr +TDG 0 hr, or +TDG 24 hr) had no significant effect (FIG. 20B), mirrored by PV FD4 levels. These results suggest that the therapeutic window of TDG treatment is at minimum within 6-12 hr after LPS treatment.

Colocalization of GLP2R with VIP and nNOS in the myenteric plexus. Immunostaining revealed that GLP2R was colocalized with nNOS and VIP in the myenteric plexus neurons and intramuscular nerve fibers in rat duodenum. Colocalization of nNOS and VIP in the myenteric neurons and nerve fibers was also confirmed. Cryostat sections of rat duodenum were immunostained with primary antibodies for GLP-2 receptor, nNOS, and VIP. These results suggest that downstream of GLP2R activation involves VIP and NO release, consistent with a prior report [11].

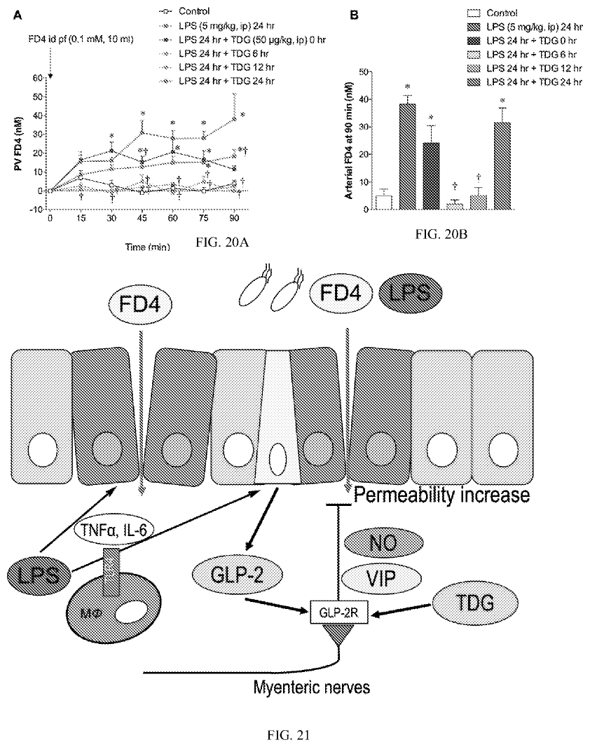

Discussion. The dynamics of small intestinal FD4 permeability into the PV after systemic LPS treatment in vivo was examined in order to test whether endogenous or exogenous GLP-2 acutely improves LPS-induced FD4 permeability and to ascertain optimal timing of treatment. The results described herein demonstrated that PV FD4 levels increased 30-90 min after FD4 perfusion into the small intestinal lumen 6 hr after LPS treatment, and further increased 24 hr after LPS treatment, that endogenous GLP-2 release was involved in LPS-induced increased FD4 permeability at least 6 hr after LPS treatment, whereas exogenous GLP-2 or the stable GLP-2 analog TDG reduced LPS-induced FD4 permeability when given 6-12 hr after LPS treatment. Total FD4 movement into the PV is closely related to arterial FD4 levels 90 min after FD4 perfusion. This is the first study showing that parenteral LPS treatment acutely releases GLP-2, which defends against LPS-induced increased small intestinal paracellular permeability via the NO and VIP pathways (FIG. 21).

There are several reasons why the FD4 solution was perfused intraduodenally and the FD4 levels in the PV was measured, rather than gavaged FD4 into conscious animals, followed by blood collection at one time point in order to assess FD4 permeability. One is that FD4 distribution into the small intestinal lumen following gavage of FD4 into the stomach is affected by gastric emptying and small intestinal motility that are prolonged during endotoxemia due to gastroparesis and paralytic ileus, potentially confounding the measurements [34]. Another is that the dynamics of PV FD4 levels is the most direct measurement of FD4 transport from the small intestinal lumen to the blood stream. Last is that GLP-2 treatment may affect FD4 movement through the stomach and small intestine, since GLP-2 relaxes gastric smooth muscle in mice and reduces antral motility in pigs [35,36], although GLP-2 or TDG has lesser or no effect on gastric emptying in humans [37,38]. Therefore, the method described herein provides the most accurate quantification available of small intestinal paracellular permeability of FD4 during LPS-induced systemic inflammation.

The results show that FD4 permeability was increased 6 hr, but not 1 or 3 hr after LPS injection. A detailed histological study of mouse small intestine demonstrated that LPS injection (10 mg/kg) increased fluid exudation and villous shortening 1.5 hr after injection with increased apoptosis and cell shedding via TLR4- and TNF receptor 1-dependent mechanisms, followed by plasma FD4 increase at 5 hr, not at 1.5 or 3 hr after injection [22], consistent with these results. Upregulation of TNFα in the ileal mucosa 6 hr after LPS treatment was also observed. These observations suggest that LPS-induced induction and release of TNFα damage villous cells with resultant epithelial gap formation and increased FD4 permeability. Upregulation of growth factors that are downstream of GLP2R activation such as EGF, IGF1, and IGF1R in the ileal mucosa of the LPS 6hr group also suggests that endogenous GLP-2/GLP2R signaling is involved in rapid mucosal repair from LPS-induced inflammation. Since LPS treatment rapidly induces villous and crypt cell apoptosis and epithelial cell shedding, followed by the increased FD4 permeability from the small intestine in mice [22], activation of the GLP2R-growth factor signal may contribute to rapid restitution of the intestinal epithelium. Interestingly, LPS-induced increased FD4 permeability at 6 hr was reversible. TDG treatment 3 and 6 hr after LPS treatment inhibited FD4 movement into the PV, suggesting that TDG acutely reverses LPS-induced FD4 permeability. GLP-2 increases release of the growth factors IGF1 and EGF. EGF, but not IGF1 promotes restitution of damaged epithelial cells within 3 hr [39]. IGF1 stimulates crypt expansion by increasing the rate of proliferation and promotes epithelial repair from enteritis in 5 days [40]. Therefore, IGF1 or EGF may be involved in the acute inhibitory effects of TDG on FD4 permeability. Nevertheless, inhibition of IGF1 or EGF tyrosine kinase failed to affect, whereas L-NAME and PG97-269 reversed the effects TDG on permeability, suggesting that the acute inhibitory effects of TDG on LPS-induced FD4 permeability are mediated by NO and VIP pathways, and not by growth factor pathways.

VIP modulates epithelial paracellular permeability via regulation of the expression and function of epithelial tight junction proteins. VIPergic pathways increase the expression of the tight junction protein zonula occludens-1 (ZO-1) peaking at 15 hr in human polarized colonic epithelial monolayers co-cultured with human submucosa containing the submucosal plexus, associated with reduced epithelial paracellular permeability [16]. VIP likely reduces FD4 flux through the epithelial monolayer in 30 min [16], suggesting that VIP rapidly regulates FD4 permeability without altering the expression of tight junctional proteins. Daily treatment with exogenous VIP also ameliorates bacterial infection-induced intestinal barrier disruption by preventing the translocation of the tight junction proteins ZO-1, occludin, and claudin-3 10 days post-infection in a Citrobacter rodentium-induced colitis model [41]. Mucosal inflammation increases epithelial paracellular permeability primarily due to the disruption of the epithelial tight junction complex by TNF-α and interferon (IFN)-γ derived from activated macrophages and T cells [42]. VIP and PACAP equally reduce TNF-α release from activated macrophages induced by LPS [43], suggesting that VIP-VPAC signaling modifies epithelial paracellular permeability changes during intestinal inflammation via inhibition of inflammatory cytokine release. Nevertheless, these studies addressed the relatively long-term effect of VIP signals rather than acute effect of VIP-VPAC signaling, which it was observed as the inhibitory effects of TDG on LPS-induced FD4 permeability that was acutely reversed by VPAC1 antagonism. The acute effect of VIP on FD4 permeability through the compromised small intestinal mucosa remains to be clarified.

NO is involved in the increase of intestinal paracellular permeability that occurs in inflammation. Excessive NO production derived from iNOS contributes to the increase of paracellular permeability 24 hr after LPS treatment (5 mg/kg) in rats, as assessed by the 3 doses of iNOS inhibitor 2, 6, and 8 hr after LPS treatment [32]. Increased FD4 flux through everted ileal sacs of LPS-treated mice at 12 hr with reduced ZO-1 expression was inhibited by the iNOS inhibition and in iNOS knockout mice, whereas iNOS gene ablation likely increases FD4 flux and reduces ZO-1 expression without LPS treatment [33], suggesting the involvement of compensatory mechanisms in iNOS knockout mice. In contrast, we observed that luminal co-perfusion of L-NAME acutely blunted the inhibitory effect of TDG on LPS-induced increased FD4 permeability at 6 hr, suggesting that NO derived from enteric nNOS by TDG treatment reduces LPS-augmented FD4 permeability, independently of iNOS-derived NO. Furthermore, luminal perfusion of L-NAME had no effect on the LPS-induced FD4 permeability increase, suggesting that the effect of L-NAME on the reversal of permeability due to TDG effect results from the inhibition of enteric nNOS rather than inhibition of iNOS. Direct and dose-dependent effects of NO on epithelial paracellular permeability remain to be determined.

The 24 hr LPS model provides a clinical correlate for the therapeutic potency of TDG. These results showed that TDG treatment inhibited LPS-induced increased FD4 permeability at 6 and 12 hr, but not 0 or 24 hr after LPS treatment, suggesting that the reversal of LPS-induced small intestinal paracellular permeability by TDG occurs within or somewhat beyond the window 6-12 hr after LPS treatment. Since the serum t_{1/2} of TDG is ˜2 hr [44], much longer than the ˜7 min t_{1/2} of exogenous GLP-2 [45], the acute effect of TDG as observed in the LPS 6 hr model may contribute to the TDG-related reversal of LPS-mediated increased intestinal permeability. These data imply that TDG may be beneficial when administered early in the course of severe inflammatory diseases such as acute pancreatitis, fulminant hepatitis, burns, and other diseases complicated by the systemic inflammatory response syndrome (SIRS), assuming that increased intestinal paracellular permeability is related to the pathogenesis of SIRS, and not merely an inflammatory biomarker [46].

In conclusion, systemic treatment with LPS releases endogenous GLP-2, which acutely preserves LPS-induced FD4 permeability in the small intestine associated with increased Gcg expression and increased GLP-2 release. Furthermore, TDG inhibits LPS-induced FD4 permeability acutely via NO and VIP-VPAC1 pathways rather than via growth factor pathways. TDG treatment may prevent the progression of intestinal barrier disruption during endotoxemia, if given at the optimal time point(s) after the induction of systemic inflammation. Exogenous GLP-2 treatment is of value in the prevention of the paracellular permeability increase associated with endotoxemia.

### REFERENCES

1 Ammori B J, Leeder P C, King R F, et al. Early increase in intestinal permeability in patients with severe acute pancreatitis: correlation with endotoxemia, organ failure, and mortality. J Gastrointest. Surg. 1999;3:252-262.

2 Gäbele E, Dostert K, Hofmann C, et al. DSS induced colitis increases portal LPS levels and enhances hepatic inflammation and fibrogenesis in experimental NASH. J Hepatol 2011;55:1391-1399.

3 Sorkine P, Szold O, Halpern P, et al. Gut decontamination reduces bowel ischemia-induced lung injury in rats. Chest 1997;112:491-495.

4 Cohen J, Vincent J L, Adhikari N K, et al. Sepsis: a roadmap for future research. Lancet Infect Dis 2015;15:581-614.

5 Drucker D J, Erlich P, Asa S L, Brubaker P L. Induction of intestinal epithelial proliferation by glucagon-like peptide 2. Proc Natl Acad Sci USA 1996;93:7911-7916.

6 Cani P D, Possemiers S, Van de W T, et al. Changes in gut microbiota control inflammation in obese mice through a mechanism involving GLP-2-driven improvement of gut permeability. Gut 2009;58:1091-1103.

7 Rowland K J, Trivedi S, Lee D, et al. Loss of glucagon-like peptide-2-induced proliferation following intestinal epithelial insulin-like growth factor-1-receptor deletion. Gastroenterology 2011;141:2166-2175.

8 Bahrami J, Yusta B, Drucker D J. ErbB activity links the glucagon-like peptide-2 receptor to refeeding-induced adaptation in the murine small bowel. Gastroenterology 2010;138:2447-2456.

9 Orskov C, Hartmann B, Poulsen S S, Thulesen J, Hare K J, Holst J J. GLP-2 stimulates colonic growth via KGF, released by subepithelial myofibroblasts with GLP-2 receptors. Regul. Pept. 2005;124:105-112.

10 Kaunitz J D, Akiba Y. Control of intestinal epithelial proliferation and differentiation: The microbiome, enteroendocrine L cells, telocytes, enteric nerves, and GLP, too. Dig Dis Sci 2019;64:2709-2716.

11 Guan X, Karpen H E, Stephens J, et al. GLP-2 receptor localizes to enteric neurons and endocrine cells expressing vasoactive peptides and mediates increased blood flow. Gastroenterology 2006;130:150-164.

12 Sugamoto S, Kawauch S, Furukawa O, Mimaki T H, Takeuchi K. Role of endogenous nitric oxide and prostaglandin in duodenal bicarbonate response induced by mucosal acidification in rats. Dig Dis Sci 2001;46:1208-1216.

13 Eklund S, Jodal M, Lundgren O, Sjoqvist A. Effects of vasoactive intestinal polypeptide on blood flow, motility and fluid transport in the gastrointestinal tract of the cat. Acta Physiol Scand 1979;105:461-468.

14 Yao B, Hogan D L, Bukhave K, Koss M A, Isenberg J I. Bicarbonate transport by rabbit duodenum in vitro: effect of vasoactive intestinal peptide, prostaglandin E2, and cyclic adenosine monophosphate. Gastroenterology 1993;104:732-740.

15 Nylander O, Hällgren A, Holm L. Duodenal mucosal alkaline secretion, permeability, and blood flow. Am J Physiol 1993;265:G1029-G1038.

16 Neunlist M, Toumi F, Oreschkova T, et al. Human ENS regulates the intestinal epithelial barrier permeability and a tight junction-associated protein ZO-1 via VIPergic pathways. Am J Physiol Gastrointest Liver Physiol 2003;285:G1028-G1036.

17 Takeuchi K, Kagawa S, Mimaki H, Aoi M, Kawauchi S. COX and NOS isoforms involved in acid-induced duodenal bicarbonate secretion in rats. Dig. Dis. Sci. 2002;47:2116-2124.

18 Kahles F, Meyer C, Mollmann J, et al. GLP-1 secretion is increased by inflammatory stimuli in an IL-6-dependent manner, leading to hyperinsulinemia and blood glucose lowering. Diabetes 2014;63:3221-3229.

19 Nguyen A T, Mandard S, Dray C, et al. Lipopolysaccharides-mediated increase in glucose-stimulated insulin secretion: involvement of the GLP-1 pathway. Diabetes 2014;63:471-482.

20 Lebrun L J, Lenaerts K, Kiers D, et al. Enteroendocrine L cells sense LPS after gut barrier injury to enhance GLP-1 secretion. Cell reports 2017;21:1160-1168.

21 Wang J H, Inoue T, Higashiyama M, et al. Umami receptor activation increases duodenal bicarbonate secretion via glucagon-like peptide-2 release in rats. J Pharmacol Exp Ther 2011;339:464-473.

22 Williams J M, Duckworth C A, Watson A J, et al. A mouse model of pathological small intestinal epithelial cell apoptosis and shedding induced by systemic administration of lipopolysaccharide. Dis Model. Mech. 2013;6:1388-1399.

23 Yue C, Wang W, Tian W L, et al. Lipopolysaccharide-induced failure of the gut barrier is site-specific and inhibitable by growth hormone. Inflamm Res 2013;62:407-415.

24 Volynets V, Reichold A, Bardos G, Rings A, Bleich A, Bischoff S C. Assessment of the intestinal barrier with five different permeability tests in healthy C57BL/6J and BALB/cJ mice. Digestive Diseases and Sciences 2016;61:737-746.

25 Gourlet P, Vandermeers A, Vertongen P, et al. Development of high affinity selective VIP1 receptor agonists. Peptides 1997;18:1539-1545.

26 Mizumori M, Meyerowitz J, Takeuchi T, et al. Epithelial carbonic anhydrases facilitate PCO₂ and pH regulation in rat duodenal mucosa. J Physiol 2006;573:827-842.

27 Akiba Y, Inoue T, Kaji I, et al. Short-chain fatty acid sensing in rat duodenum. J Physiol 2015;593:585-599.

28 Thulesen J, Knudsen L B, Hartmann B, et al. The truncated metabolite GLP-2 (3-33) interacts with the GLP-2 receptor as a partial agonist. Regul Pept 2002;103:9-15.

29 Drucker D J, DeForest L, Brubaker P L. Intestinal response to growth factors administered alone or in combination with human [Gly2]glucagon-like peptide 2. Am J Physiol 1997;273 :G1252-G1262.

30 Garcia-Echeverria C, Pearson M A, Marti A, et al. In vivo antitumor activity of NVP-AEW541-A novel, potent, and selective inhibitor of the IGF-IR kinase. Cancer Cell 2004;5:231-239.

31 Bos M, Mendelsohn J, Kim Y M, Albanell J, Fry D W, Baselga J. PD153035, a tyrosine kinase inhibitor, prevents epidermal growth factor receptor activation and inhibits growth of cancer cells in a receptor number-dependent manner. Clin Cancer Res 1997;3:2099-2106.

32 Unno N, Wang H, Menconi M J, et al. Inhibition of inducible nitric oxide synthase ameliorates endotoxin-induced gut mucosal barrier dysfunction in rats. Gastroenterology 1997;113:1246-1257.

33 Han X, Fink M P, Yang R, Delude R L. Increased iNOS activity is essential for intestinal epithelial tight junction dysfunction in endotoxemic mice. Shock 2004;21:261-270.

34 Buchholz B M, Chanthaphavong R S, Bauer A J. Nonhemopoietic cell TLR4 signaling is critical in causing early lipopolysaccharide-induced ileus. J Immunol 2009;183:6744-6753.

35 Amato A, Baldassano S, Serio R, Mule F. Glucagon-like peptide-2 relaxes mouse stomach through vasoactive intestinal peptide release. Am J Physiol Gastrointest Liver Physiol 2009;296:G678-G684.

36 Wøjdemann M, Wettergren A, Hartmann B, Hoist J J. Glucagon-like peptide-2 inhibits centrally induced antral motility in pigs. Scand J Gastroenterol 1998;33:828-832.

37 Nagell C F, Wettergren A, Pedersen J F, Mortensen D, Holst J J. Glucagon-like peptide-2 inhibits antral emptying in man, but is not as potent as glucagon-like peptide-1. Scand J Gastroenterol 2004;39:353-358.

38 Berg J K, Kim E H, Li B, Joelsson B, Youssef N N. A randomized, double-blind, placebo-controlled, multiple-dose, parallel-group clinical trial to assess the effects of teduglutide on gastric emptying of liquids in healthy subjects. BMC Gastroenterol 2014;14:25.

39 Riegler M, Sedivy R, Sogukoglu T, et al. Epidermal growth factor promotes rapid response to epithelial injury in rabbit duodenum in vitro. Gastroenterology 1996;111:28-36.

40 Van Landeghem L, Santoro M A, Mah A T, et al. IGF1 stimulates crypt expansion via differential activation of 2 intestinal stem cell populations. FASEB J2015;29:2828-2842.

41 Conlin V S, Wu X, Nguyen C, et al. Vasoactive intestinal peptide ameliorates intestinal barrier disruption associated with Citrobacter rodentium-induced colitis. Am J Physiol Gastrointest Liver Physiol 2009;297:G735-750.

42 Clayburgh D R, Barrett T A, Tang Y, et al. Epithelial myosin light chain kinase-dependent barrier dysfunction mediates T cell activation-induced diarrhea in vivo. J Clin Invest 2005;115:2702-2715.

43 Delgado M, Pozo D, Martinez C, et al. Vasoactive intestinal peptide and pituitary adenylate cyclase-activating polypeptide inhibit endotoxin-induced TNF-alpha production by macrophages: in vitro and in vivo studies. J Immunol 1999;162:2358-2367.

44 Marier J F, Mouksassi M S, Gosselin N H, Beliveau M, Cyran J, Wallens J. Population pharmacokinetics of teduglutide following repeated subcutaneous administrations in healthy participants and in patients with short bowel syndrome and Crohn's disease. J Clin Pharmacol 2010;50:36-49.

45 Hansen L, Hare K J, Hartmann B, et al. Metabolism of glucagon-like peptide-2 in pigs: role of dipeptidyl peptidase IV. Regul Pept 2007;138:126-132.

46 Hollander D, Kaunitz J D. The “Leaky Gut”: Tight junctions but loose associations? Dig Dis Sci 2019;(in press).

1. A method of suppressing or preventing systemic organ inflammation in a human patient with acute illness, the method comprising: (a) identifying a human patient in need of treatment; and (b) administering to the human patient a therapeutically effective amount of a pharmaceutical composition comprising glucagon-like peptide (GLP)-2 or a GLP-2 analog; and a pharmaceutically acceptable carrier.

2. A method of suppressing or preventing systemic organ inflammation in a human patient with an acute inflammatory disorder associated with systemic inflammatory response syndrome (SIRS) or multiorgan failure, the method comprising: (a) identifying a human patient in need of treatment; and (b) administering to the human patient a therapeutically effective amount of a pharmaceutical composition comprising glucagon-like peptide (GLP)-2 or a GLP-2 analog; and a pharmaceutically acceptable carrier.

3. (canceled)

4. The method of claim 1, wherein the GLP2 or GLP-2 analog is administered between 1 second and 12 hours before systemic inflammation is induced.

5. The method of claim 1, wherein the GLP-2 analog is teduglutide.

6. The method of claim 1, wherein the acute illness is burns, major surgery, sepsis, an autoimmune disorder, vasculitis, thromboembolism, trauma or acute pancreatitis.

7. (canceled)

8. The method of claim 5, wherein the amount of teduglutide administered to the patient ranges from 0.05 to 1 mg/kg per day.

9. (canceled)

10. (canceled)

11. The method claim 1, wherein the pharmaceutical composition is administered through intravenous infusion.

12. (canceled)

13. (canceled)

14. The method of claim 11, wherein the continuous intravenous infusion occurs over a period of at least 24 hours to about 7 days.

15. (canceled)

16. The method of claim 1, wherein the systemic organ inflammation suppressed or prevented is in the patient's liver, lungs, kidneys, brain, hematopoietic system, gastrointestinal system, blood coagulation system, vascular system or a combination thereof.

17. The method of claim 1, wherein the systemic organ inflammation is suppressed or prevented by reducing expression or preventing an increase in the expression of one or more cytokines.

18. The method of claim 17, wherein the one or more cytokines are tumor necrosis factor alpha, interleukin-1β or interleukin-6.

19. The method of claim 1, wherein the systemic organ inflammation is suppressed or prevented by preventing an increase in lipopolysaccharide concentrations in the patient's portal venous blood.

20-35. (canceled)

36. A method of preventing or reducing endotoxin entry into a human patient's portal vein, the method comprising: (a) identifying the human patient in need of treatment; and (b) administering to the human patient a therapeutically effective amount of a pharmaceutical composition comprising GLP-2 or a GLP-2;
and a pharmaceutically acceptable carrier.

37. The method of claim 36, wherein the GLP2 or GLP-2 analog is administered between 1 second and 12 hours before systemic inflammation is induced.

38. The method of claim 36, wherein the GLP-2 analog is teduglutide.

39. (canceled)

40. The method of claim 38, wherein the amount of teduglutide administered to the patient ranges from 0.05 to 1 mg/kg/day per day.

41. (canceled)

42. (canceled)

43. The method of claim 36, wherein the pharmaceutical composition is administered through intravenous infusion.

44. (canceled)

45. (canceled)

46. The method of claim 43, wherein the continuous intravenous infusion occurs over a period of at least 24 hours to about 7 days.

47. (canceled)

48. The method of claim 36, wherein endotoxin entry into the portal vein is reduced or prevented or prevented in the patient's liver or lungs thereby reducing expression or preventing an increase in the expression of one or more cytokines.

49. The method of claim 48, wherein the one or more cytokines are tumor necrosis factor alpha, interleukin-1β or interleukin-6.

50-59. (canceled)


SEQUENCE LISTING (41 sequences)

SEQ ID NO: 1 - protein, 33 aa, Artificial Sequence
  Synthetic construct
HADGSFSDEMNTILDNLAARDFINWLIQTKITD

SEQ ID NO: 2 - protein, 34 aa, Artificial Sequence
  Synthetic construct
HADGSFSDEMNTILDNLAARDFINWLIQTKITDR

SEQ ID NO: 3 - protein, 33 aa, Artificial Sequence
  Synthetic construct
HADGSFSDEMNTILDNLATRDFINWLIQTKITD

SEQ ID NO: 4 - protein, 33 aa, Artificial Sequence
  Synthetic construct
YADGSFSDEMNTILDNLATRDFINWLIQTKITD

SEQ ID NO: 5 - protein, 33 aa, Artificial Sequence
  Synthetic construct
HGDGSFSDEMNTILDNLATRDFINWLIQTKITD

SEQ ID NO: 6 - protein, 33 aa, Artificial Sequence
  Synthetic construct
HVDGSFSDEMNTILDNLATRDFINWLIQTKITD

SEQ ID NO: 7 - protein, 33 aa, Artificial Sequence
  Synthetic construct
HGDGSFSDEMNTILDNLAARDFINWLIQTKITD

SEQ ID NO: 8 - protein, 33 aa, Artificial Sequence
  Synthetic construct
HGDGSFSDEMNTILDNLAAADFINWLIQTKITD

SEQ ID NO: 9 - protein, 33 aa, Artificial Sequence; identical to SEQ ID NO: 3
  Synthetic construct
  MISC_FEATURE (2)..(2): D configuration

SEQ ID NO: 10 - protein, 33 aa, Artificial Sequence
  Synthetic construct
AGDGSFSDEMNTILDNLAARDFINWLIQTKITD

SEQ ID NO: 11 - protein, 33 aa, Artificial Sequence
  Synthetic construct
HGAGSFSDEMNTILDNLAARDFINWLIQTKITD

SEQ ID NO: 12 - protein, 33 aa, Artificial Sequence
  Synthetic construct
HGDASFSDEMNTILDNLAARDFINWLIQTKITD

SEQ ID NO: 13 - protein, 33 aa, Artificial Sequence
  Synthetic construct
HAEGSFSDEMNTILDNLATRDFINWLIQTKITD

SEQ ID NO: 14 - protein, 33 aa, Artificial Sequence
  Synthetic construct
HADASFSDEMNTILDNLATRDFINWLIQTKITD

SEQ ID NO: 15 - protein, 33 aa, Artificial Sequence
  Synthetic construct
  MISC_FEATURE (13)..(13): Ile13=desIle
HADGSFSDYSKYILDNLAARDFINWLIQTKITD

SEQ ID NO: 16 - protein, 33 aa, Artificial Sequence
  Synthetic construct
HADGSFSDELNTILDNLATRDFINWLIQTKITD

SEQ ID NO: 17 - protein, 33 aa, Artificial Sequence; identical to SEQ ID NO: 16
  Synthetic construct
  MISC_FEATURE (10)..(10): Norleucine

SEQ ID NO: 18 - protein, 33 aa, Artificial Sequence; identical to SEQ ID NO: 3
  Synthetic construct
  MOD_RES (10)..(10): SULFATATION

SEQ ID NO: 19 - protein, 33 aa, Artificial Sequence
  Synthetic construct
HADGSFSDEMNTILDNLATKDFINWLIQTKITD

SEQ ID NO: 20 - protein, 33 aa, Artificial Sequence
  Synthetic construct
HADGSFSDEMNTILDNLAARDFVQWLIQTKITD

SEQ ID NO: 21 - protein, 33 aa, Artificial Sequence; identical to SEQ ID NO: 1
  Synthetic construct
  MOD_RES (33)..(33): AMIDATION

SEQ ID NO: 22 - protein, 33 aa, Artificial Sequence
  Synthetic construct
HGDGSFSDEMNTILDNLATRDFIAWLIQTKITD

SEQ ID NO: 23 - protein, 33 aa, Artificial Sequence
  Synthetic cosntruct
HGDGSFSAEMNTILDNLATRDFINWLIQTKITD

SEQ ID NO: 24 - protein, 33 aa, Artificial Sequence
  Synthetic construct
HGDGSFSDEMATILDNLATRDFINWLIQTKITD

SEQ ID NO: 25 - protein, 33 aa, Artificial Sequence
  Synthetic construct
HGDGSFSDEMNTILDNLATRAFINWLIQTKITD

SEQ ID NO: 26 - protein, 33 aa, Artificial Sequence
  Synthetic construct
HGDGSFSDAMNTILDNLATRDFINWLIQTKITD

SEQ ID NO: 27 - protein, 33 aa, Artificial Sequence
  Synthetic construct
HGDGSFSDEMNTILDALATRDFINWLIQTKITD

SEQ ID NO: 28 - protein, 33 aa, Artificial Sequence
  Synthetic construct
HGDGSFSDEMNTILDNAATRDFINWLIQTKITD

SEQ ID NO: 29 - protein, 33 aa, Artificial Sequence
  Synthetic construct
HGDGSFSDEMNTILDNLATRDFINWLIATKITD

SEQ ID NO: 30 - protein, 33 aa, Artificial Sequence
  Synthetic construct
HGDGAFSDEMNTILDNLATRDFINWLIQTKITD

SEQ ID NO: 31 - protein, 33 aa, Artificial Sequence
  Synthetic construct
HGDGSFSDEMNTILDNLATRDFINWLIQTKATD

SEQ ID NO: 32 - protein, 33 aa, Artificial Sequence
  Synthetic construct
HGDGSFSDEMNTILDNLATRDFINWLAQTKITD

SEQ ID NO: 33 - protein, 33 aa, Artificial Sequence
  Synthetic construct
HGDGSFSDEMNAILDNLATRDFINWLIQTKITD

SEQ ID NO: 34 - protein, 33 aa, Artificial Sequence
  Synthetic construct
HGDGSFSDEMNTALDNLATRDFINWLIQTKITD

SEQ ID NO: 35 - protein, 33 aa, Artificial Sequence
  Synthetic construct
HGDGSFADEMNTILDNLATRDFINWLIQTKITD

SEQ ID NO: 36 - protein, 33 aa, Artificial Sequence
  Synthetic construct
HGDGSASDEMNTILDNLATRDFINWLIQTKITD

SEQ ID NO: 37 - protein, 33 aa, Artificial Sequence; identical to SEQ ID NO: 1
  Synthetic construct

SEQ ID NO: 38 - protein, 33 aa, Artificial Sequence
  Synthetic construct
  MISC_FEATURE (10)..(10): Norleucine
  MISC_FEATURE (11)..(11): D configuration
HGDGSFSDELFTILDLLAARDFINWLIQTKITD

SEQ ID NO: 39 - protein, 39 aa, Artificial Sequence
  Synthetic construct
HGEGTFSSELATILDALAARDFIAWLIATKITDKKKKKK

SEQ ID NO: 40 - protein, 33 aa, Artificial Sequence
  Synthetic construct
HGDGSFSDEANTILDNLAARDFINWLIQTKITD

SEQ ID NO: 41 - protein, 27 aa, Artificial Sequence
  Synthetic construct
  MOD_RES (1)..(1): ACETYLATION
  MISC_FEATURE (2)..(2): D configuration
  MOD_RES (27)..(27): AMIDATION
HFDAVFTNSYRKVLKRLSARKLLQDIL